(19) United States
(12) Patent Application Publication
(10) Pub. No.: US 2025/0118441 A1
(43) Pub. Date: Apr. 10, 2025

(54) SNP-BASED MODEL FOR PREDICTING OCCURRENCE OF IMMUNE-RELATED ADVERSE EVENT INDUCED BY IMMUNO THERAPY

(71) Applicants: THE ASAN FOUNDATION, Seoul (KR); UNIVERSITY OF ULSAN FOUNDATION FOR INDUSTRY COOPERATION, Ulsan (KR); KOREA ADVANCED INSTITUTE OF SCIENCE AND TECHNOLOGY, Daejeon (KR)

(72) Inventors: Sook Ryun PARK, Seoul (KR); Jung Kyoon CHOI, Daejeon (KR); Jinhyeon AN, Daejeon (KR); Changhwan SUNG, Daejeon (KR)

(73) Assignees: THE ASAN FOUNDATION, Seoul (KR); UNIVERSITY OF ULSAN FOUNDATION FOR INDUSTRY COOPERATION, Ulsan (KR); KOREA ADVANCED INSTITUTE OF SCIENCE AND TECHNOLOGY, Daejeon (KR)

(21) Appl. No.: 18/730,634

(22) PCT Filed: Jan. 20, 2023
(86) PCT No.: PCT/KR2023/001078

§ 371 Date: Jul. 19, 2024

(30) Foreign Application Priority Data
Jan. 20, 2022 (KR) 10-2022-0008320
Jan. 19, 2023 (KR) 10-2023-0008358

(51) Int. Cl. G16H 50/30; G16B 20/20; G16B 40/20; G16H 10/40

(52) U.S. Cl. CPC G16H 50/30; G16B 20/20; G16B 40/20; G16H 10/40

(57) ABSTRACT

A SNP-based model for predicting the occurrence of irAEs induced by immunotherapy. The dbSNP database rs541169 and a single nucleotide polymorphism (SNP) site in the TMEM162 (FAM187B) gene are closely correlated with the occurrence of irAEs. The rs541169 can be advantageously used as a biomarker for predicting the occurrence of irAEs induced by immunotherapy or predicting responsiveness to immunotherapy.

### TECHNICAL FIELD

The present invention relates to an SNP-based model for predicting the occurrence of an immunotherapy-induced immune-related adverse event.

This application claims priority to and the benefit of Korean Patent Application No. 10-2022-0008320, filed on Jan. 20, 2022, and Korean Patent Application No. 10-2023-0008358, filed on Jan. 19, 2023, the disclosures of which are incorporated herein by reference in their entirety.

### BACKGROUND ART

Immune checkpoint blockade (ICB) treatment has become one of the main treatments for various cancer types, and has expanded its role from adjuvant therapy to the neoadjuvant setting due to immune-related adverse events (irAEs) following ICB treatment. Although most early-stage or low-grade irAEs can be managed with corticosteroids or immunosuppressants, some irAEs can be fatal or leave permanent morbidity if not detected and treated promptly. Therefore, the prediction of irAEs occurrence before ICB pre-treatment (PRE) or early during treatment (EDT) is of great clinical importance not only in terms of patient management but also in terms of healthcare costs. In addition, irAEs provide the opportunity to understand how autoimmunity develops in response to an immune activator in general.

Previous irAEs studies have primarily focused on clinical or biochemical features measured in peripheral blood. Although the complete blood count (CBC) has been studied extensively, several studies have produced conflicting results, and these discrepancies indicate that CBC-based biomarkers are easily influenced by factors unrelated to tumors, such as a patient's clinical status and medical history. Cytokine profile has also been proposed as a predictor of irAEs. For example, IL-6 inhibits the differentiation of regulatory T cells and B cells and contributes to overactivation of the adaptive immune system, and clonal expansion of CD8+ T cells in peripheral blood was associated with severe irAEs occurrence in patients treated with ipilimumab.

Separate from peripheral blood measurement, tumor mutation burden has been proposed as an indicator of irAE incidence in an attempt to explain the relatively high irAE incidence in lung cancer and melanoma. However, tumor mutation burden is likely to act as a confounding factor that indirectly increases the risk of irAEs by promoting treatment response to ICB. In addition, through TCGA multi-omics data analysis, LCP1 and ADPGK were identified as predictive biomarkers for irAEs, but validation of the predictive power was performed with a limited number of lung cancer patients comparing 14 irAE samples with 14 control samples. These two studies relied on data from the FDA Adverse Event Reporting System (FAERS), but this database was not specifically designed to study ICB-associated irAEs. For germline variation, multigenetic risk scores obtained from genome-wide association studies were applied to atezolizumab-induced skin- or thyroid-associated irAEs.

That is, the genetic, molecular and cellular risk factors for irAEs are difficult to identify and require integrated analysis, and the diversity of irAEs pathology implies a multifaceted complexity of the fundamental mechanisms and requires a much more comprehensive investigation. However, most of the previous irAEs studies were limited to specific drugs (e.g., ipilimumab or atezolizumab), irAEs conditions (e.g., autoimmune ty in the skin), and cancer types (e.g., lung cancer or melanoma), often using a limited number of irAEs samples.

Accordingly, the present inventions integrated multidimensional data, including genetic factors, molecular and cellular profiles of immune cells, laboratory data, and clinical variables before and after ICB treatment for hundreds of patients with various types of irAEs to perform comprehensive analysis of irAEs, to provide a biomarker and a method for predicting the irAEs occurrence induced by cancer immunotherapy, such as ICB occurrence.

### DISCLOSURE

### Technical Problem

The present inventors analyzed genetic factors, molecular and cellular profiles of immune cells, laboratory data and clinical variables for the occurrence of irAEs before and after ICB treatment and confirmed a correlation between single nucleotide polymorphism (SNP) in TMEM162 (FAM187B) gene and the occurrence of irAEs. Based on this, the present invention was completed.

Therefore, the present invention is directed to providing a composition and kit for predicting the occurrence of an cancer immunotherapy-irAEs, which includes an agent for detecting the SNP in dbSNP database rs541169.

The present invention is also directed to providing a method of providing information for predicting the occurrence of a cancer immunotherapy-induced irAEs or a method of providing information for predicting the responsiveness to a cancer immunotherapy, which includes detecting SNP in dbSNP database rs541169 in a biological sample isolated from a subject.

However, the technical problem to be achieved by the present invention is not limited to the problems mentioned above, and other problems not mentioned can be clearly understood by those of ordinary skill in the art from the description below.

### Technical Solution

To achieve the above-mentioned purposes, the present invention provides a composition for predicting the occurrence of cancer immunotherapy-induced irAEs, which includes an agent for detecting SNP in dbSNP database rs541169.

The present invention also provides a kit for predicting the occurrence of cancer immunotherapy-induced irAEs, which includes the composition.

The present invention also provides a method of providing information for predicting the occurrence of cancer immunotherapy-induced irAEs, which includes detecting SNP in dbSNP database rs541169 in a biological sample isolated from a subject.

The present invention also provides a method of predicting information for predicting the responsiveness to a cancer immunotherapy, which includes detecting SNP in dbSNP database rs541169 in a biological sample isolated from a subject.

The present invention also provides a method of predicting the occurrence of cancer immunotherapy-induced irAEs, which includes detecting SNP in dbSNP database rs541169 in a biological sample isolated from a subject.

The present invention also provides a method of predicting the responsiveness to a cancer immunotherapy, which includes detecting SNP in dbSNP database rs541169 in a biological sample isolated from a subject.

In one embodiment of the present invention, the detection agent may be to detect a variation in which a nucleotide of dbSNP database rs541169 is T, but the present invention is not limited thereto.

In another embodiment of the present invention, the SNP of rs541169 may cause the cleavage of TMEM162 protein, but the present invention is not limited thereto.

In still another embodiment of the present invention, the detection agent may be a primer or probe that can detect rs541169 SNP, but the present invention is not limited thereto.

In yet another embodiment of the present invention, irAEs may be one or more selected from the group consisting of a skin adverse event (Skin), an endocrine system adverse event (Endocrine), a thyroid gland adverse event (Thyroid), a musculoskeletal system adverse event (Musculoskeletal), a gastrointestinal system adverse event (Gastrointestinal), a neurologic system adverse event (Neurologic), a flu-like symptom (Flu-like), and pneumonia (Pulmonary), which occur due to a cancer immunotherapy, but the present invention is not limited thereto.

In yet another embodiment of the present invention, the composition may further include an agent for detecting one or more SNPs in the dbSNP database listed in the following table, but the present invention is not limited thereto.

| Type of irAE | SNV ID | rsID | mean_abs_shap | stdev_abs_shap | Annotated gene |
|---|---|---|---|---|---|
| Any | chr17_76187108_G_C | rs77585764 | 0.029 | 0.036 | AFMID |
| Any | chr21_37661405_G_A | rs3827183 | 0.028 | 0.009 | DOPEY2 |
| Any | chr4_187629770_A_C | rs3733414 | 0.033 | 0.009 | FAT1 |
| Any | chr4_103611845_C_T | rs227368 | 0.035 | 0.018 | MANBA |
| Any | chr11_1972205_G_A | rs2240197 | 0.033 | 0.015 | MRPL23 |
| Any | chr8_52733050_T_A | rs12335014 | 0.039 | 0.014 | PCMTD1 |
| Any | chr16_72156842_T_G | rs16973716 | 0.046 | 0.007 | PMFBP1 |
| Any | chr10_134646988_C_T | rs4880433 | 0.029 | 0.004 | TTC40 |
| Any | chr13_32885654_T_C | rs445909 | 0.032 | 0.012 | ZAR1L |
| Critical | chr10_24908686_T_C | rs3748222 | 0.023 | 0.008 | ARHGAP21 |
| Critical | chr12_13526328_G_T | rs17821405 | 0.024 | 0.017 | C12orf36 |
| Critical | chr13_113909339_A_G | rs2302757 | 0.021 | 0.007 | CUL4A |
| Critical | chr21_37661405_G_A | rs3827183 | 0.021 | 0.012 | DOPEY2 |
| Critical | chr11_73988167_C_T | rs2282488 | 0.028 | 0.004 | P4HA3 |
| Critical | chr18_10731428_C_T | rs2865121 | 0.016 | 0.004 | PIEZO2 |
| Critical | chr8_10390452_C_T | rs4406360 | 0.022 | 0.014 | PRSS55 |
| Critical | chr1_28282292_T_C | rs3813803 | 0.024 | 0.000 | SMPDL3B |
| Critical | chr22_50964236_G_A | rs11479 | 0.023 | 0.000 | TYMP |
| Critical | chr1_108307727_T_A | rs7528153 | 0.017 | 0.006 | VAV3 |
| Endocrine | chr7_150733025_T_A | rs2303926 | 0.025 | 0.001 | ABCB8 |
| Endocrine | chr20_31688241_T_C | rs11699009 | 0.029 | 0.002 | BPIFB4 |
| Endocrine | chr21_43523941_C_A | rs220111 | 0.026 | 0.005 | C21orf128 |
| Endocrine | chr8_25364834_A_T | rs3829009 | 0.021 | 0.001 | CDCA2 |
| Endocrine | chr13_50467044_G_C | rs554324677 | 0.021 | 0.008 | CTAGE10P |
| Endocrine | chr6_46563817_A_T | rs7761731 | 0.030 | 0.003 | CYP39A1 |
| Endocrine | chr11_19735987_A_G | rs2278132 | 0.022 | 0.002 | LOC100126784 |
| Endocrine | chr8_52733050_T_A | rs12335014 | 0.026 | 0.013 | PCMTD1 |
| Endocrine | chr20_44141331_A_G | rs6032259 | 0.029 | 0.000 | SPINT3 |
| Endocrine | chr19_56703248_G_A | rs527025 | 0.023 | 0.000 | ZSCAN5B |
| Flu-like | chr4_91230579_G_A | rs12647859 | 0.027 | 0.003 | CCSER1 |
| Flu-like | chr8_25364834_A_T | rs3829009 | 0.031 | 0.001 | CDCA2 |
| Flu-like | chr5_13894894_T_A | rs4701997 | 0.030 | 0.006 | DNAH5 |
| Flu-like | chr2_39008949_G_A | rs1056104 | 0.026 | 0.001 | GEMIN6 |
| Flu-like | chr1_79095581_T_C | rs987495 | 0.028 | 0.004 | IFI44L |
| Flu-like | chr10_22839628_T_C | rs2230469 | 0.028 | 0.014 | PIP4K2A |
| Flu-like | chr11_94322352_A_T | rs11020845 | 0.028 | 0.005 | PIWIL4 |
| Flu-like | chr2_95947099_G_A | rs72819488 | 0.026 | 0.008 | PROM2 |
| Flu-like | chr1_227935762_G_A | rs2236358 | 0.036 | 0.006 | SNAP47 |
| Flu-like | chr17_5058808_G_A | rs9899177 | 0.029 | 0.010 | USP6 |
| Gastrointestinal | chr5_114607217_T_A | rs77125878 | 0.020 | 0.010 | CCDC112 |
| Gastrointestinal | chr2_202939654_T_C | rs10221698 | 0.018 | 0.008 | LOC100652824 |
| Gastrointestinal | chr21_16339570_T_A | rs2228507 | 0.019 | 0.008 | NRIP1 |
| Gastrointestinal | chr19_50412650_C_T | rs3745489 | 0.017 | 0.009 | NUP62 |
| Gastrointestinal | chr12_57619362_G_A | rs10783816 | 0.021 | 0.002 | NXPH4 |
| Gastrointestinal | chr6_144081609_C_T | rs2073214 | 0.032 | 0.008 | PHACTR2 |
| Gastrointestinal | chr10_3200292_G_A | rs3182535 | 0.020 | 0.008 | PITRM1 |
| Gastrointestinal | chr8_26365716_C_T | rs2233701 | 0.023 | 0.003 | PNMA2 |
| Gastrointestinal | chr8_10467589_T_C | rs9657518 | 0.023 | 0.013 | RP1L1 |
| Gastrointestinal | chr13_41382711_A_T | rs17849654 | 0.018 | 0.000 | SLC25A15 |
| Musculoskeletal | chr2_241451351_G_A | rs35044862 | 0.020 | 0.002 | ANKMY1 |
| Musculoskeletal | chr5_131822072_A_G | rs2070724 | 0.018 | 0.003 | C5orf56 |
| Musculoskeletal | chr19_45016116_A_G | rs8100718 | 0.020 | 0.003 | CEACAM20 |
| Musculoskeletal | chr5_118506685_G_A | rs9790916 | 0.017 | 0.004 | DMXL1 |
| Musculoskeletal | chr4_22389797_A_C | rs3814416 | 0.017 | 0.012 | GPR125 |
| Musculoskeletal | chr5_118811533_G_A | rs25640 | 0.022 | 0.004 | HSD17B4 |
| Musculoskeletal | chr19_35719106_A_G | rs565791 | 0.015 | 0.003 | NXPH4 |
| Musculoskeletal | chr1_67109335_A_G | rs7526812 | 0.017 | 0.006 | SGIP1 |
| Musculoskeletal | chr17_7606722_C_G | rs7640 | 0.020 | 0.003 | WRAP53 |
| Musculoskeletal | chr7_64863392_C_T | rs10265083 | 0.019 | 0.006 | ZNF92 |
| Multiple (any grade) | chr4_114278277_C_T | rs3733617 | 0.029 | 0.016 | ANK2 |
| Multiple (any grade) | chr12_48888594_C_T | rs11458 | 0.034 | 0.016 | C12orf54 |
| Multiple (any grade) | chr18_25616451_A_T | rs1944294 | 0.028 | 0.005 | CDH2 |
| Multiple (any grade) | chr19_41117869_A_G | rs1131620 | 0.028 | 0.009 | LTBP4 |
| Multiple (any grade) | chr7_99696797_T_C | rs2070215 | 0.041 | 0.007 | MCM7 |
| Multiple (any grade) | chr12_57619362_G_A | rs10783816 | 0.032 | 0.009 | NXPH4 |
| Multiple (any grade) | chr17_3119767_C_T | rs769427 | 0.032 | 0.016 | OR1A1 |
| Multiple (any grade) | chr6_144081609_C_T | rs2073214 | 0.028 | 0.012 | PHACTR2 |
| Multiple (any grade) | chr12_14720554_G_T | rs1141509 | 0.046 | 0.008 | PLBD1 |
| Multiple (any grade) | chr1_248039451_C_T | rs3811444 | 0.028 | 0.007 | TRIM58 |
| Multiple (grade ≥2) | chr11_134856603_C_T | rs117318814 | 0.017 | 0.000 | AK130852 |
| Multiple (grade ≥2) | chr4_114278277_C_T | rs3733617 | 0.017 | 0.001 | ANK2 |
| Multiple (grade ≥2) | chr2_241451351_G_A | rs35044862 | 0.013 | 0.001 | ANKMY1 |
| Multiple (grade ≥2) | chr18_76829525_A_G | rs4078115 | 0.017 | 0.003 | ATP9B |
| Multiple (grade ≥2) | chr5_114607217_T_A | rs77125878 | 0.014 | 0.011 | CCDC112 |
| Multiple (grade ≥2) | chr5_118506685_G_A | rs9790916 | 0.019 | 0.002 | DMXL1 |
| Multiple (grade ≥2) | chr21_37617724_C_A | rs3746866 | 0.015 | 0.004 | DOPEY2 |
| Multiple (grade ≥2) | chr4_89577214_A_T | rs2972040 | 0.015 | 0.001 | HERC3 |
| Multiple (grade ≥2) | chr5_7870973_A_G | rs1801394 | 0.013 | 0.004 | MTRR |
| Multiple (grade ≥2) | chr11_48238549_G_A | rs11606506 | 0.020 | 0.007 | OR4B1 |
| Neurologic | chr3_100558416_T_C | rs9841585 | 0.016 | 0.005 | AB13BP |
| Neurologic | chr19_5772897_C_T | rs78536254 | 0.020 | 0.000 | CATSPERD |
| Neurologic | chr5_118506685_G_A | rs9790916 | 0.017 | 0.002 | DMXL1 |
| Neurologic | chr2_234750542_G_C | rs3732215 | 0.023 | 0.002 | HJURP |
| Neurologic | chr1_6157434_C_T | rs2311802 | 0.020 | 0.013 | KCNAB2 |
| Neurologic | chr13_44457925_A_G | rs3764147 | 0.019 | 0.004 | LACC1 |
| Neurologic | chr1_222803204_A_G | rs2936051 | 0.015 | 0.002 | MIA3 |
| Neurologic | chr15_42143533_T_G | rs1197665 | 0.018 | 0.001 | SPTBN5 |
| Neurologic | chr16_2812939_C_A | rs2240140 | 0.016 | 0.006 | SRRM2 |
| Neurologic | chr17_15611495_T_C | rs3760299 | 0.015 | 0.007 | ZNF286A |
| Pulmonary | chr6_109894773_T_A | rs12175588 | 0.004 | 0.002 | AK9 |
| Pulmonary | chr2_228493211_G_A | rs28739019 | 0.004 | 0.001 | C2orf83 |
| Pulmonary | chr14_24679877_C_T | rs2295322 | 0.004 | 0.000 | CHMP4A |
| Pulmonary | chr2_220037666_A_G | rs1127102 | 0.004 | 0.001 | CNPPD1 |
| Pulmonary | chr14_55655692_C_T | rs2274271 | 0.004 | 0.004 | DLGAP5 |
| Pulmonary | chr19_54758504_C_G | rs117301131 | 0.005 | 0.004 | LILRB5 |
| Pulmonary | chr8_125568175_T_C | rs3793395 | 0.004 | 0.002 | MTSS1 |
| Pulmonary | chr14_60585131_A_G | rs308998 | 0.004 | 0.000 | PCNXL4 |
| Pulmonary | chr7_140080087_C_G | rs62490396 | 0.006 | 0.001 | SLC37A3 |
| Pulmonary | chr8_135649848_G_A | rs12541381 | 0.005 | 0.001 | ZFAT |
| Skin | chr7_150733025_T_A | rs2303926 | 0.056 | 0.001 | ABCB8 |
| Skin | chr12_52306221_C_T | rs2277382 | 0.034 | 0.036 | ACVRL1 |
| Skin | chr15_34159986_T_C | rs2241647 | 0.043 | 0.014 | AVEN |
| Skin | chr14_74489735_G_A | rs3784039 | 0.049 | 0.058 | CCDC176 |
| Skin | chr3_128204693_G_C | rs78245253 | 0.035 | 0.043 | GATA2 |
| Skin | chr9_712156_T_G | rs912174 | 0.051 | 0.018 | KANK1 |
| Skin | chr19_51361472_C_A | rs2003783 | 0.039 | 0.025 | KLK3 |
| Skin | chr12_31288955_G_A | rs11051266 | 0.035 | 0.002 | OVOS2 |
| Skin | chr1_22793576_G_A | rs2236358 | 0.051 | 0.009 | SNAP47 |

In yet another embodiment of the present invention, the method may further include predicting that the risk of the occurrence of cancer immunotherapy-induced irAEs is high when a variation in which a nucleotide of dbSNP database rs541169 is T is detected in a biological sample isolated from a subject, but the present invention is not limited thereto.

In yet another embodiment of the present invention, the method may further include measuring one or more activities selected from the group consisting of B cells, regulatory T cells, and exhausted T cells in a biological sample isolated from a subject; and
predicting that the risk of the occurrence of cancer immunotherapy-induced irAEs is high when the B cell activity is relatively high, or the regulatory T cell or exhausted T cell activity is relatively low, but the present invention is not limited thereto.

In the present invention, the biological sample may be one or more selected from the group consisting of tissue, cells, whole blood, serum, plasma, saliva, sputum, cerebrospinal fluid, urine, and feces, which are isolated from a subject, and according to one example or experimental example of the present invention, the biological sample may be whole blood, but the present invention is not limited thereto.

The present invention also provides a use of an agent for detecting SNP in dbSNP database rs541169 or a composition including the same to predict the occurrence of cancer immunotherapy-induced irAEs or the responsiveness to a cancer immunotherapy.

The present invention also provides a use of an agent for detecting SNP in dbSNP database rs541169 or a composition including the same to prepare an agent for predicting the occurrence of cancer immunotherapy-induced irAEs or to prepare an agent for predicting the responsiveness to a cancer immunotherapy.

### Advantageous Effects

As a result of analyzing various factors associated with the occurrence of irAEs induced by a cancer immunotherapy in the present invention, it was confirmed that dbSNP database rs541169, which is the SNP site in TMEM162 (FAM187B) gene, is closely associated with the occurrence of irAEs. Therefore, the rs541169 is expected to be useful as a biomarker for predicting the occurrence of cancer immunotherapy-induced irAEs or the responsiveness to a cancer immunotherapy.

### DESCRIPTION OF DRAWINGS

FIG. 1A is the view that shows cohort data summarization and data predictive modeling workflow according to one embodiment of the present invention.

FIG. 1B is the density plot that shows various types of irAEs occurrence over time after ICB administration according to one embodiment of the present invention.

FIG. 1C is the view that shows the network analysis of various types of irAEs according to one embodiment of the present invention.

FIG. 1D is the PCA analysis results for cell type abundance in whole blood RNA-seq data according to one embodiment of the present invention.

FIG. 2A is the laboratory data result related to various types of irAEs occurrence in ICB PRE and EDT patient samples according to one embodiment of the present invention.

FIG. 2B is the view that confirms the correlation between complete blood count (CBC) neutrophil count and neutrophil cell fractions estimated based on RNA sequencing data inference in PRE and EDT patient samples according to one embodiment of the present invention.

FIG. 2C is the view that compares the expression levels of neutrophil marker genes between irAEs and a control in PRE and EDT patient samples according to one embodiment of the present invention (*P<0.05, **P<0.01, ***P<0.001).

FIG. 2D shows the volcano plot of genes that are differentially expressed between irAEs and a control in PRE and EDT patient samples according to one embodiment of the present invention.

FIG. 2E is the view that shows the result of analyzing pathway enrichment for underexpressed genes in irAEs in PRE and EDT patient samples according to one embodiment of the present invention.

FIG. 2F is the view that confirms the degree of pathway enrichment for underexpressed genes in 12 irAEs types in PRE and EDT patient samples according to one embodiment of the present invention.

FIG. 3A is the view that confirms the volcano plot of genes that are differentially expressed between irAEs and a control according to one embodiment of the present invention.

FIG. 3B is the view that confirms the expression of ANKRD22 gene according to the type of immune cells according to one embodiment of the present invention.

FIG. 3C is the view that confirms the result of analyzing pathway enrichment associated with neutrophil-related function and cytokine-mediated signal regulation between irAEs and a control in EDT patient samples according to one embodiment of the present invention.

FIG. 3D is the view that shows the result of analyzing pathway enrichment for a gene up-regulated in response to ICB only in irAEs or a control according to one embodiment of the present invention.

FIG. 3E is the view that confirms changes in the expression of neutrophil activity-related genes, CLEC4D and CAMP, and changes in the expression of NK cell activity-related genes, CD160 and KLRC1, between irAEs and a control in PRE and EDT patient samples according to one embodiment of the present invention.

FIG. 4A is the view that compares the proportion of the CNV status of HLA-B exon 2 in irAEs and a control according to one embodiment of the present invention.

FIG. 4B is the view that shows the result of analyzing irAEs cumulative incidence according to the CNV status of HLA-B exon 2 according to one embodiment of the present invention.

FIG. 4C is the view that confirms the copy number of HLA-B exon 2 in irAEs and a control according to one embodiment of the present invention.

FIG. 4D is the view that confirms the association between main HLA alleles and 12 irAEs types by heatmap analysis according to one embodiment of the present invention.

FIG. 4E is the view that confirms the proportions of alleles showing excellent correlation among the main HLA alleles in irAE and a control according to one embodiment of the present invention.

FIG. 4F is the view that shows the proportions of 12 irAEs types according to HLA-B*35:01 allele possession according to one embodiment of the present invention.

FIG. 5A is the view that shows the correlation matrix between 29 types of irAEs based on the association with common SNVs according to one embodiment of the present invention.

FIG. 5B is the view that confirms the correlation between 12 irAEs types and various laboratory data, and CNV and SNV properties by heatmap analysis according to one embodiment of the present invention.

FIG. 5C is the view that confirms the performance of prospective models for 12 irAEs types based on average precision, accuracy, and AUC according to one embodiment of the present invention.

FIG. 5D is the view that shows the result of Manhattan plots for SNVs and CNVs related to all irAEs types according to one embodiment of the present invention.

FIG. 6A is the view that confirms the derived allele frequency of rs541169 depending on population type of various countries according to one embodiment of the present invention.

FIG. 6B is the view that confirms the result of analyzing irAEs cumulative incidence due to the presence of a rs541169 mutant according to one embodiment of the present invention.

FIG. 6C is the view that shows the proportions of 12 irAEs types according to genotype of rs541169 according to one embodiment of the present invention.

FIG. 6D is the view that shows the proportions of 12 irAEs types due to the presence of a rs541169 mutant according to one embodiment of the present invention.

FIG. 6E is the view that shows the expression level of TMEM162 gene in various tissues according to one embodiment of the present invention.

FIG. 6F is the view that confirms the expression levels of TMEM162 and BTN2A1 genes in various tissues according to one embodiment of the present invention.

FIG. 6G is the view that confirms the fractions of immune cell types according to rs541169 genotypes in PRE and EDT patient samples according to one embodiment of the present invention.

FIG. 6H is the view that confirms the fractions of regulatory T (Treg) cells according to rs541169 genotypes in PRE and EDT patient samples according to one embodiment of the present invention.

FIG. 6I is the view that confirms the fractions of exhausted T (Tex) cells according to rs541169 genotypes in PRE and EDT patient samples according to one embodiment of the present invention.

FIG. 6J is the view that confirms immune signature scores according to rs541169 genotypes in the immune clusters of TCGA pan-cancer samples according to one embodiment of the present invention.

FIG. 6K is the view that shows the results of an HKA test around rs541169 in the 1,094 Korean whole genome sequences according to one embodiment of the present invention.

FIG. 6L is the view that confirms the HKA values for rs541169 depending on population type of various countries according to one embodiment of the present invention.

### BEST MODE

The present invention provides a composition for predicting the occurrence of cancer immunotherapy-induced irAEs, which includes an agent for detecting SNP in dbSNP database rs541169.

In the present invention, “polymorphism” used herein refers to the generation of two or more alternative sequences or alleles in a genetically determined population, “single nucleotide polymorphism (SNP)” refers to the polymorphism of one base. Specifically, the polymorphism refers to the single nucleotide (A, T, C, or G) variation of a DNA sequence in a genome level, caused between members of species or between pairs of chromosomes in an individual. For example, when including a difference in single nucleotide like three DNA fragments (e.g., AAGT[A/A]AG, AAGT[A/G]AG, and AAGT[G/G]AG) of different individuals, it is called two alleles (A or G), generally, almost all SNPs have two alleles. In addition, when SNP is closely genetically linked to a specific disease, the SNP also indicates that a variation has occurred in one base at a specific site compared to a confirmed normal or wild-type (WT) individual or allele. In the present invention, “single nucleotide variant (SNV)” refers to a variant showing a difference in single nucleotide, and the variation in one nucleotide at a specific site comparing a normal or wild-type (WT) subject or allele.

In the present invention, the composition detects SNP in dbSNP database rs541169 as a diagnosis marker.

In the present invention, the SNP rs541169 may be a variation of C to G or T of the base 35228117 on human chromosome 19, and according to one example or experimental example of the present invention, it may be a variation of C to T (C>T), but the present invention is not limited thereto.

In the present invention, “cancer immunotherapy” used herein refers to a drug that strengthens the inherent immune system of a human body and increases its resistance to cancer. A cancer immunotherapy has fewer side effects in that it is used to treat a patient by strengthening the patient's own immunity, and improves the quality of life of a cancer patient, and significantly extends survival time. A cancer immunotherapy exhibits an anticancer effect by strengthening the specificity, memory, and adaptiveness of the immune system. A cancer immunotherapy is, for example, an agent for immune checkpoint blockade (ICB), immune cell therapy, therapeutic antibody, or immune checkpoint enhancer, but is not limited to its type. In the present invention, unlike existing immunotherapies (a cytokine treatment, an anti-cancer vaccine, etc.), the agent for ICB, i.e., an immune checkpoint inhibitor binds to the binding sites of cancer cells and T cells and blocks immune evasion signals, thereby preventing the formation of an immunological synapse, and thus has a mechanism by which T cells that are not hindered by immune evasion destroy cancer cells, and may be, for example, one or more selected from the group consisting of nivolumab, atezolizumab, pembrolizumab, duvalumab, avelumab, ipilimumab, and tremelimumab, but the present invention is not limited thereto.

In the present invention, “immune-related adverse event (irAE)” refers to a variety of adverse events caused by treatment with a cancer immunotherapy, including inflammatory responses that occur in relation to the activation of an autoimmune system.

In the present invention, the irAEs may be one or more selected from the group consisting of a skin adverse event (Skin), an endocrine system adverse event (Endocrine), a thyroid gland adverse event (Thyroid), a musculoskeletal system adverse event (Musculoskeletal), a gastrointestinal system adverse event (Gastrointestinal), a neurologic system adverse event (Neurologic), a flu-like symptom (Flu-like), and pneumonia (Pulmonary), which occur due to a cancer immunotherapy, but the present invention is not limited thereto. According to one example or experimental example of the present invention, the irAEs may be accompanied by two or more symptoms, and their severity may be divided into three grades. For example, in the present invention, when there are three or more types of irAEs depending on the severity of the irAEs, the result is shown as ‘Multiple G>=1’, when there are three or more types of grade 2 or higher, the result is shown as ‘Multiple G>=2’, when there are any type of grade 3 or higher and a critical type of grade 2 or higher, the result is shown as ‘Critical’, and when there is any type included in the category of irAEs, it may be shown as ‘Any’.

In the present invention, “detection” encompasses both identifying and confirming the presence (expression) of a target material, and measuring and confirming a change in the level of existence (expression level) of a target material. In the same context, in the present invention, detecting SNP in dbSNP database rs541169 means determining whether SNP is expressed (i.e., verifying the presence or absence of SNP), or measuring the qualitative, quantitative change levels of the SNP in dbSNP database rs541169. The measurement includes both qualitative and quantitative methods (analyses) and can be performed without limitation. The types of qualitative and quantitative methods for identifying the presence of SNPs are well known in the art, and include the experimental methods described herein.

In the present invention, the detection agent may be to detect a variation in which a nucleotide of dbSNP database rs541169 is changed to T, but the present invention is not limited thereto.

In the present invention, the rs541169 SNP may cause the cleavage of TMEM162 (FAM187B) protein, but the present invention is not limited thereto.

In the present invention, the detection agent may be a primer or probe that can detect rs541169 SNP, but the present invention is not limited thereto.

In the present invention, “primer” is a short single strand oligonucleotide that acts as a starting point of DNA synthesis. A primer specifically binds to a polynucleotide, which is a template, in appropriate buffer and under temperature conditions, and DNA polymerase allows a nucleoside triphosphate having a complementary base to the template DNA to be linked to the primer, resulting in synthesizing DNA. A primer generally consists of the sequence of 15 to 30 bases, and a melting temperature (Tm) at which the bases bind to the template strand varies depending on base composition and the length of the primer. The sequence of a primer does not need to have a sequence that is perfectly complementary to a part of the base sequence of the template, but it is sufficient as long as the primer has a length and complementarity suitable for the purpose of measuring the amount of mRNA by amplifying a specific section of mRNA or cDNA through DNA synthesis. Therefore, in the present invention, primer pairs can be easily designed by referring to the base sequence of the cDNA or genomic DNA of the gene or mRNA thereof. Primers for the amplification reaction consist of a set (pair) that binds complementary to the template (or sense) and opposite side (antisense) of the both ends of a specific section of mRNA to be amplified.

In the present invention, “probe” refers to a fragment of a polynucleotide, such as RNA or DNA with a length of at least several to maximum hundreds of base pairs that can specifically bind to mRNA, complementary DNA (cDNA), or DNA of a specific gene, and may be labeled to confirm the presence or absence and the expression level of the binding target mRNA or cDNA. Conditions for probe selection and hybridization may be appropriately selected according to techniques known in the art. Such a probe may be used in a diagnosis method for detecting an allelomorphic trait. The diagnosis method may include detection methods based on the hybridization of nucleic acids, such as Southern blotting, and a probe may be provided as previously binding to a substrate of a DNA chip in a method using a DNA chip.

In the present invention, the primer or probe may be chemically synthesized using a phosphoramidite solid support synthesis method or other widely known methods. In addition, the primer or probe may be modified in various forms by a method known in the art without interfering with hybridization with a polynucleotide, which becomes a target to be detected. Examples of such modifications include methylation, capping, substitution with one or more homologs of a natural nucleotide, modification between nucleotides, such as uncharged linkers (e.g., methyl phosphonate, phosphotriester, phosphoramidate, carbamate, etc.), or charged linkers (e.g., phosphorothioate, phosphorodithioate, etc.), and binding of a labeling material using fluorescence or an enzyme.

In the present invention, the primer or probe is not limited to a specific sequence as long as it can detect rs541169 SNP.

The present invention also provides a kit for predicting the occurrence of cancer immunotherapy-induced irAEs, which includes the composition.

In the present invention, a “kit” refers to a tool that include an agent for detecting the rs541169 SNP, and thus can predict the occurrence of cancer immunotherapy-induced irAEs or the responsiveness to a cancer immunotherapy of a cancer patient. The kit of the present invention may include other components, compositions, solutions, or devices, which are conventionally required for methods for measuring or detecting them, in addition to the above-described agent. Here, the material for measuring the rs541169 SNP may be applied one or more times without limit, and there is no limit to the timing of the application of each material, and the application of each material may be done simultaneously or at different times.

In the present invention, the kit may include a container; instructions; and an agent for measuring the rs541169 SNP. The container may serve to package the agent, and also serve to store and fix it. The material of the container may be provided in a form, such as a bottle, a tub, a sachet, an envelope, a tube, or an ampoule, and may be formed partially or entirely from plastic, glass, paper, foil, or wax. The container may be equipped with a completely or partially removable closure, which may initially be part of the container or may be attached to the container by mechanical, adhesive, or other means, or may be equipped with a stopper that can access to the contents by a needle. The kit may include an external package, and the external package may include instructions on the use of components.

The present invention also provides a method providing information for predicting the occurrence of cancer immunotherapy-induced irAEs or a method of predicting the occurrence of cancer immunotherapy-induced irAEs, which includes detecting SNP in dbSNP database rs541169 in a biological sample isolated from a subject.

The present invention also provides a method of providing information for predicting the responsiveness to a cancer immunotherapy or a method of predicting the responsiveness to a cancer immunotherapy, which includes detecting SNP in dbSNP database rs541169 in a biological sample isolated from a subject.

In the present invention, “subject” may include both cancer patients to be treated with a cancer immunotherapy and cancer patients who had been treated with a cancer immunotherapy. Here, the subject may be mammals, including a human or non-human primate, a mouse, a rate, a dog, a cat, a horse, and a cow, but the present invention is not limited thereto.

In the present invention, the cancer may be one or more selected from the group consisting of lung cancer including non-small cell lung cancer and small cell lung cancer, esophageal cancer, hepatocellular carcinoma, stomach cancer, breast cancer, bladder cancer, kidney cancer, bile duct cancer, urethral cancer, head and neck cancer, melanoma, colon cancer, gallbladder cancer, pancreatic cancer, and ampulla of Vater cancer, neuroendocrine carcinoma, paraganglioma, ovarian cancer, uterine cancer, prostate cancer, thymic cancer, and cerebral hemangiosarcoma, but the present invention is not limited thereto.

In the present invention, the biological sample may be one or more selected from the group consisting of tissue, cells, whole blood, serum, plasma, saliva, sputum, cerebrospinal fluid, urine, and feces, which are isolated from a subject, and according to one example or experimental example of the present invention, the biological sample may be whole blood, but the present invention is not limited thereto.

In the present invention, the method may further include predicting that the risk of the occurrence of cancer immunotherapy-induced irAEs is high when a variation in which a nucleotide of dbSNP database rs541169 is T in a biological sample isolated from a subject, but the present invention is not limited thereto.

In the present invention, the composition may further include an agent for detecting one or more SNPs of the SNPs of dbSNP database listed in Table 8 of the present invention, and preferably, an agent for detecting one or more SNPs of the SNPs of the dbSNP database listed in the following Table, but the present invention is not limited thereto.

In the present invention, the method may further include detecting one or more SNPs of the SNPs of dbSNP database listed in Table 8 of the present invention, and preferably, detecting one or more SNPs of the SNPs of dbSNP database listed in the following Table, but the present invention is not limited thereto.

| Type of irAE | SNV ID | rsID | mean_abs_shap | stdev_abs_shap | Annotated gene |
|---|---|---|---|---|---|
| Any | chr17_76187108_G_C | rs77585764 | 0.029 | 0.036 | AFMID |
| Any | chr21_37661405_G_A | rs3827183 | 0.028 | 0.009 | DOPEY2 |
| Any | chr4_187629770_A_C | rs3733414 | 0.033 | 0.009 | FAT1 |
| Any | chr4_103611845_C_T | rs227368 | 0.035 | 0.018 | MANBA |
| Any | chr11_1972205_G_A | rs2240197 | 0.033 | 0.015 | MRPL23 |
| Any | chr8_52733050_T_A | rs12335014 | 0.039 | 0.014 | PCMTD1 |
| Any | chr16_72156842_T_G | rs16973716 | 0.046 | 0.007 | PMFBP1 |
| Any | chr10_134646988_C_T | rs4880433 | 0.029 | 0.004 | TTC40 |
| Any | chr13_32885654_T_C | rs445909 | 0.032 | 0.012 | ZAR1L |
| Critical | chr10_24908686_T_C | rs3748222 | 0.023 | 0.008 | ARHGAP21 |
| Critical | chr12_13526328_G_T | rs17821405 | 0.024 | 0.017 | C12orf36 |
| Critical | chr13_113909339_A_G | rs2302757 | 0.021 | 0.007 | CUL4A |
| Critical | chr21_37661405_G_A | rs3827183 | 0.021 | 0.012 | DOPEY2 |
| Critical | chr11_73988167_C_T | rs2282488 | 0.028 | 0.004 | P4HA3 |
| Critical | chr18_10731428_C_T | rs2865121 | 0.016 | 0.004 | PIEZO2 |
| Critical | chr8_10390452_C_T | rs4406360 | 0.022 | 0.014 | PRSS55 |
| Critical | chr1_28282292_T_C | rs3813803 | 0.024 | 0.000 | SMPDL3B |
| Critical | chr22_50964236_G_A | rs11479 | 0.023 | 0.000 | TYMP |
| Critical | chr1_108307727_T_A | rs7528153 | 0.017 | 0.006 | VAV3 |
| Endocrine | chr7_150733025_T_A | rs2303926 | 0.025 | 0.001 | ABCB8 |
| Endocrine | chr20_31688241_T_C | rs11699009 | 0.029 | 0.002 | BPIFB4 |
| Endocrine | chr21_43523941_C_A | rs220111 | 0.026 | 0.005 | C21orf128 |
| Endocrine | chr8_25364834_A_T | rs3829009 | 0.021 | 0.001 | CDCA2 |
| Endocrine | chr13_50467044_G_C | rs554324677 | 0.021 | 0.008 | CTAGE10P |
| Endocrine | chr6_46563817_A_T | rs7761731 | 0.030 | 0.003 | CYP39A1 |
| Endocrine | chr11_19735987_A_G | rs2278132 | 0.022 | 0.002 | LOC100126784 |
| Endocrine | chr8_52733050_T_A | rs12335014 | 0.026 | 0.013 | PCMTD1 |
| Endocrine | chr20_44141331_A_G | rs6032259 | 0.029 | 0.000 | SPINT3 |
| Endocrine | chr19_56703248_G_A | rs527025 | 0.023 | 0.000 | ZSCAN5B |
| Flu-like | chr4_91230579_G_A | rs12647859 | 0.027 | 0.003 | CCSER1 |
| Flu-like | chr8_25364834_A_T | rs3829009 | 0.031 | 0.001 | CDCA2 |
| Flu-like | chr5_13894894_T_A | rs4701997 | 0.030 | 0.006 | DNAH5 |
| Flu-like | chr2_39008949_G_A | rs1056104 | 0.026 | 0.001 | GEMIN6 |
| Flu-like | chr1_79095581_T_C | rs987495 | 0.028 | 0.004 | IFI44L |
| Flu-like | chr10_22839628_T_C | rs2230469 | 0.028 | 0.014 | PIP4K2A |
| Flu-like | chr11_94322352_A_T | rs11020845 | 0.028 | 0.005 | PIWIL4 |
| Flu-like | chr2_95947099_G_A | rs72819488 | 0.026 | 0.008 | PROM2 |
| Flu-like | chr1_227935762_G_A | rs2236358 | 0.036 | 0.006 | SNAP47 |
| Flu-like | chr17_5058808_G_A | rs9899177 | 0.029 | 0.010 | USP6 |
| Gastrointestinal | chr5_114607217_T_A | rs77125878 | 0.020 | 0.010 | CCDC112 |
| Gastrointestinal | chr2_202939654_T_C | rs10221698 | 0.018 | 0.008 | LOC100652824 |
| Gastrointestinal | chr21_16339570_T_A | rs2228507 | 0.019 | 0.008 | NRIP1 |
| Gastrointestinal | chr19_50412650_C_T | rs3745489 | 0.017 | 0.009 | NUP62 |
| Gastrointestinal | chr12_57619362_G_A | rs10783816 | 0.021 | 0.002 | NXPH4 |
| Gastrointestinal | chr6_144081609_C_T | rs2073214 | 0.032 | 0.008 | PHACTR2 |
| Gastrointestinal | chr10_3200292_G_A | rs3182535 | 0.020 | 0.008 | PITRM1 |
| Gastrointestinal | chr8_26365716_C_T | rs2233701 | 0.023 | 0.003 | PNMA2 |
| Gastrointestinal | chr8_10467589_T_C | rs9657518 | 0.023 | 0.013 | RP1L1 |
| Gastrointestinal | chr13_41382711_A_T | rs17849654 | 0.018 | 0.000 | SLC25A15 |
| Musculoskeletal | chr2_241451351_G_A | rs35044862 | 0.020 | 0.002 | ANKMY1 |
| Musculoskeletal | chr5_131822072_A_G | rs2070724 | 0.018 | 0.003 | C5orf56 |
| Musculoskeletal | chr19_45016116_A_G | rs8100718 | 0.020 | 0.003 | CEACAM20 |
| Musculoskeletal | chr5_118506685_G_A | rs9790916 | 0.017 | 0.004 | DMXL1 |
| Musculoskeletal | chr4_22389797_A_C | rs3814416 | 0.017 | 0.012 | GPR125 |
| Musculoskeletal | chr5_118811533_G_A | rs25640 | 0.022 | 0.004 | HSD17B4 |
| Musculoskeletal | chr19_35719106_A_G | rs565791 | 0.015 | 0.003 | NXPH4 |
| Musculoskeletal | chr1_67109335_A_G | rs7526812 | 0.017 | 0.006 | SGIP1 |
| Musculoskeletal | chr17_7606722_C_G | rs7640 | 0.020 | 0.003 | WRAP53 |
| Musculoskeletal | chr7_64863392_C_T | rs10265083 | 0.019 | 0.006 | ZNF92 |
| Multiple (any grade) | chr4_114278277_C_T | rs3733617 | 0.029 | 0.016 | ANK2 |
| Multiple (any grade) | chr12_48888594_C_T | rs11458 | 0.034 | 0.016 | C12orf54 |
| Multiple (any grade) | chr18_25616451_A_T | rs1944294 | 0.028 | 0.005 | CDH2 |
| Multiple (any grade) | chr19_41117869_A_G | rs1131620 | 0.028 | 0.009 | LTBP4 |
| Multiple (any grade) | chr7_99696797_T_C | rs2070215 | 0.041 | 0.007 | MCM7 |
| Multiple (any grade) | chr12_57619362_G_A | rs10783816 | 0.032 | 0.009 | NXPH4 |
| Multiple (any grade) | chr17_3119767_C_T | rs769427 | 0.032 | 0.016 | OR1A1 |
| Multiple (any grade) | chr6_144081609_C_T | rs2073214 | 0.028 | 0.012 | PHACTR2 |
| Multiple (any grade) | chr12_14720554_G_T | rs1141509 | 0.046 | 0.008 | PLBD1 |
| Multiple (any grade) | chr1_248039451_C_T | rs3811444 | 0.028 | 0.007 | TRIM58 |
| Multiple (grade ≥2) | chr11_134856603_C_T | rs117318814 | 0.017 | 0.000 | AK130852 |
| Multiple (grade ≥2) | chr4_114278277_C_T | rs3733617 | 0.017 | 0.001 | ANK2 |
| Multiple (grade ≥2) | chr2_241451351_G_A | rs35044862 | 0.013 | 0.001 | ANKMY1 |
| Multiple (grade ≥2) | chr18_76829525_A_G | rs4078115 | 0.017 | 0.003 | ATP9B |
| Multiple (grade ≥2) | chr5_114607217_T_A | rs77125878 | 0.014 | 0.011 | CCDC112 |
| Multiple (grade ≥2) | chr5_118506685_G_A | rs9790916 | 0.019 | 0.002 | DMXL1 |
| Multiple (grade ≥2) | chr21_37617724_C_A | rs3746866 | 0.015 | 0.004 | DOPEY2 |
| Multiple (grade ≥2) | chr4_89577214_A_T | rs2972040 | 0.015 | 0.001 | HERC3 |
| Multiple (grade ≥2) | chr5_7870973_A_G | rs1801394 | 0.013 | 0.004 | MTRR |
| Multiple (grade ≥2) | chr11_48238549_G_A | rs11606506 | 0.020 | 0.007 | OR4B1 |
| Neurologic | chr3_100558416_T_C | rs9841585 | 0.016 | 0.005 | AB13BP |
| Neurologic | chr19_5772897_C_T | rs78536254 | 0.020 | 0.000 | CATSPERD |
| Neurologic | chr5_118506685_G_A | rs9790916 | 0.017 | 0.002 | DMXL1 |
| Neurologic | chr2_234750542_G_C | rs3732215 | 0.023 | 0.002 | HJURP |
| Neurologic | chr1_6157434_C_T | rs2311802 | 0.020 | 0.013 | KCNAB2 |
| Neurologic | chr13_44457925_A_G | rs3764147 | 0.019 | 0.004 | LACC1 |
| Neurologic | chr1_222803204_A_G | rs2936051 | 0.015 | 0.002 | MIA3 |
| Neurologic | chr15_42143533_T_G | rs1197665 | 0.018 | 0.001 | SPTBN5 |
| Neurologic | chr16_2812939_C_A | rs2240140 | 0.016 | 0.006 | SRRM2 |
| Neurologic | chr17_15611495_T_C | rs3760299 | 0.015 | 0.007 | ZNF286A |
| Pulmonary | chr6_109894773_T_A | rs12175588 | 0.004 | 0.002 | AK9 |
| Pulmonary | chr2_228493211_G_A | rs28739019 | 0.004 | 0.001 | C2orf83 |
| Pulmonary | chr14_24679877_C_T | rs2295322 | 0.004 | 0.000 | CHMP4A |
| Pulmonary | chr2_220037666_A_G | rs1127102 | 0.004 | 0.001 | CNPPD1 |
| Pulmonary | chr14_55655692_C_T | rs2274271 | 0.004 | 0.004 | DLGAP5 |
| Pulmonary | chr19_54758504_C_G | rs117301131 | 0.005 | 0.004 | LILRB5 |
| Pulmonary | chr8_125568175_T_C | rs3793395 | 0.004 | 0.002 | MTSS1 |
| Pulmonary | chr14_60585131_A_G | rs308998 | 0.004 | 0.000 | PCNXL4 |
| Pulmonary | chr7_140080087_C_G | rs62490396 | 0.006 | 0.001 | SLC37A3 |
| Pulmonary | chr8_135649848_G_A | rs12541381 | 0.005 | 0.001 | ZFAT |
| Skin | chr7_150733025_T_A | rs2303926 | 0.056 | 0.001 | ABCB8 |
| Skin | chr12_52306221_C_T | rs2277382 | 0.034 | 0.036 | ACVRL1 |
| Skin | chr15_34159986_T_C | rs2241647 | 0.043 | 0.014 | AVEN |
| Skin | chr14_74489735_G_A | rs3784039 | 0.049 | 0.058 | CCDC176 |
| Skin | chr3_128204693_G_C | rs78245253 | 0.035 | 0.043 | GATA2 |
| Skin | chr9_712156_T_G | rs912174 | 0.051 | 0.018 | KANK1 |
| Skin | chr19_51361472_C_A | rs2003783 | 0.039 | 0.025 | KLK3 |
| Skin | chr12_31288955_G_A | rs11051266 | 0.035 | 0.002 | OVOS2 |
| Skin | chr1_22793576_G_A | rs2236358 | 0.051 | 0.009 | SNAP47 |

In the present invention, the method may further include measuring one or more activities selected from the group consisting of B cells, regulatory T cells, and exhausted T cells in a biological sample isolated from a subject; and
predicting that the risk of the occurrence of cancer immunotherapy-induced irAEs is high when the B cell activity is relatively high, or the activity of the regulatory T cells or exhausted T cells is relatively low, but the present invention is not limited thereto.

That is, in the present invention, when the variation in which a base of rs541169 is Tis detected, and the B cell activity is relatively high or the activity of the regulatory T cells or exhausted T cells is relatively low, it may be predicted that the risk of the occurrence of cancer immunotherapy-induced irAEs is higher or the responsiveness to a cancer immunotherapy is lower, but the present invention is not limited thereto.

In the present invention, the detecting of dbSNP database rs541169 SNP may be performed using a conventional method known in the art, for example, one or more selected from the group consisting of sequencing, exome sequencing, next generation sequencing (NGS), pyrosequencing, microarray hybridization, allele-specific PCR, dynamic allele-specific hybridization, PCR extension analysis, a PCR-SSCP method, and a Taqman technique, but the present invention is not limited thereto.

In addition, the present invention provides a method of providing information for predicting the occurrence of cancer immunotherapy-induced irAEs or a method of providing information for predicting the responsiveness to a cancer immunotherapy, which includes detecting one or more selected from the group consisting of a neutrophil count, a neutrophil-to-lymphocyte ratio (NLR), a lymphocyte count, and a platelet-to-lymphocyte ratio (PLR) in biological samples isolated from a subject.

In the present invention, when the neutrophil count, NLR, or PLR is relatively low; or when the lymphocyte count is relatively high, it may be expected to have a higher risk of the occurrence of cancer immunotherapy-induced irAEs or a lower responsiveness to a cancer immunotherapy, but the present invention is not limited thereto.

In the present invention, the neutrophil count, NLR, lymphocyte count, or PLR may be used as a single model for predicting the occurrence of cancer immunotherapy-induced irAEs or predicting the responsiveness to a cancer immunotherapy, or may be also used with rs541169 SNP, but the present invention is not limited thereto.

In addition, the present invention provides a use of an agent for detecting SNP in dbSNP database rs541169 or a composition including the same to predict the occurrence of cancer immunotherapy-induced irAEs; or to predict the responsiveness to a cancer immunotherapy.

In addition, the present invention provides a use of an agent for detecting SNP in dbSNP database rs541169 or a composition including the same to prepare an agent for predicting the occurrence of cancer immunotherapy-induced irAEs; or to prepare an agent for predicting the responsiveness to a cancer immunotherapy.

In addition, the present invention provides a method of providing information for determining/analyzing a subject with high susceptibility to the prediction of the occurrence of a cancer immunotherapy-induced irAEs or the prediction of the responsiveness to a cancer immunotherapy, which includes, when a variation in which a nucleotide of rs541169 is T is detected by detecting SNPs of dbSNP database rs541169 from biological samples isolated from a subject suspected of having a high risk of the occurrence of irAEs or having a low responsiveness to a cancer immunotherapy after treatment of cancer patients with a cancer immunotherapy, determining that the resulting sample is a subject having a high risk of the occurrence of irAEs or a subject having a low responsiveness to a cancer immunotherapy after treatment with the cancer immunotherapy.

When the term “including or comprising” used herein is used, it means that other components are further included, not excluding other components unless specifically stated otherwise. The term “step” or “stage” of something used throughout the present invention does not mean a step for something.

Hereinafter, preferred examples are presented to allow the present invention to be better understood. However, the following examples are merely provided to more easily understand the present invention, and the content of the present invention is not limited by the following examples.

### EXAMPLES

### Example 1. Multi-Center Prospective Cohort of Immune-Related Adverse Events (irAEs)

A total of 672 patient consisting of 372 irAE patients treated with immune checkpoint blockage (ICB) treatment and 300 non-irAE patients, enrolled at Asan Medical Center in Seoul, were recruited to conduct a study.

The basic characteristics of the patient cohort, including patients' age, sex, cancer type, and type of ICB treatment administered, are shown in Table 1 below.

TABLE 1

| | | | | | non-irAE | |
|---|---|---|---|---|---|---|
| | Total | | irAE group | | group | |
| | (N = 672) | | (N = 372) | | (N = 300) | |
| Age | | | | | | |
| Median, years | 63 | (24-89) | 62 | (24-87) | 64 | (26-89) |
| (range) | | | | | | |
| >=65 | 298 | (44.3%) | 153 | (41.1%) | 145 | (48.3%) |
| <65 | 374 | (55.7%) | 219 | (58.9%) | 155 | (51.7%) |
| Sex | | | | | | |
| Male | 490 | (72.9%) | 275 | (73.9%) | 215 | (71.7%) |
| Female | 182 | (27.1%) | 97 | (26.1%) | 85 | (28.3%) |
| Cancer type | | | | | | |
| NSCLC | 257 | (38.2%) | 134 | (36.0%) | 123 | (41.0%) |
| Esophageal cancer | 71 | (10.6%) | 52 | (14.0%) | 19 | (6.3%) |
| HCC or combined | 65 | (9.7%) | 37 | (9.9%) | 28 | (9.3%) |
| HCC-CCA | | | | | | |
| Gastric cancer | 62 | (9.2%) | 37 | (9.9%) | 25 | (8.3%) |
| Breast cancer | 34 | (5.1%) | 29 | (7.8%) | 5 | (1.7%) |
| Bladder cancer | 33 | (4.9%) | 13 | (3.5%) | 20 | (6.7%) |
| Renal cancer | 27 | (4.0%) | 15 | (4.0%) | 12 | (4.0%) |
| Biliary cancer | 23 | (3.4%) | 10 | (2.7%) | 13 | (4.3%) |
| Ureter cancer | 22 | (3.3%) | 10 | (2.7%) | 12 | (4.0%) |
| Head and neck | 20 | (3.0%) | 8 | (2.2%) | 12 | (4.0%) |
| cancer | | | | | | |
| Melanoma | 16 | (2.4%) | 5 | (1.3%) | 11 | (3.7%) |
| Small cell | 13 | (1.9%) | 4 | (1.1%) | 9 | (3.0%) |
| lung cancer | | | | | | |
| Colorectal cancer | 11 | (1.6%) | 11 | (3.0%) | 0 | (0.0%) |
| Gall bladder cancer | 5 | (0.7%) | 2 | (0.5%) | 3 | (1.0%) |
| Pancreas cancer | 2 | (0.3%) | 1 | (0.3%) | 1 | (0.3%) |
| Ampulla of | 2 | (0.3%) | 1 | (0.3%) | 1 | (0.3%) |
| vater cancer | | | | | | |
| Cancer of unknown | 2 | (0.3%) | 1 | (0.3%) | 1 | (0.3%) |
| primary site | | | | | | |
| Neuroendocrine | 1 | (0.1%) | 0 | (0.0%) | 1 | (0.3%) |
| carcinoma | | | | | | |
| Paraganglioma | 1 | (0.1%) | 1 | (0.3%) | 0 | (0.0%) |
| Ovarian cancer | 1 | (0.1%) | 0 | (0.0%) | 1 | (0.3%) |
| Uterine cancer | 1 | (0.1%) | 0 | (0.0%) | 1 | (0.3%) |
| Prostate cancer | 1 | (0.1%) | 0 | (0.0%) | 1 | (0.3%) |
| Thymic carcinoma | 1 | (0.1%) | 1 | (0.3%) | 0 | (0.0%) |
| Angiosarcoma | 1 | (0.1%) | 0 | (0.0%) | 1 | (0.3%) |
| of brain | | | | | | |
| ICB type* | | | | | | |
| Nivolumab | 217 | (32.3%) | 131 | (35.2%) | 86 | (28.7%) |
| Atezolizumab | 193 | (28.7%) | 92 | (24.7%) | 101 | (33.7%) |
| Pembrolizumab | 151 | (22.5%) | 81 | (21.8%) | 70 | (23.3%) |
| Duvalumab | 36 | (5.4%) | 23 | (6.2%) | 13 | (4.3%) |
| Avelumab | 3 | (0.4%) | 2 | (0.5%) | 1 | (0.3%) |
| Nivolumab + | 12 | (1.8%) | 8 | (2.2%) | 4 | (1.3%) |
| Ipilimumab | | | | | | |
| Duvalumab + | 1 | (0.1%) | 1 | (0.3%) | 0 | (0.0%) |
| Tremelimumab | | | | | | |
| Investigational | 72 | (10.7%) | 47 | (12.6%) | 25 | (8.3%) |
| Immune checkpoint | | | | | | |
| treatment | | | | | | |
| ICB treatment | | | | | | |
| regimen* | | | | | | |
| ICB monotherapy | 573 | (85.3%) | 310 | (83.3%) | 263 | (87.7%) |
| ICB + ICB | 15 | (2.2%) | 9 | (2.4%) | 6 | (2.0%) |
| ICB + cytotoxic | 65 | (9.7%) | 43 | (11.6%) | 22 | (7.3%) |
| chemotherapy | | | | | | |
| ICB + molecular | 12 | (1.8%) | 5 | (1.3%) | 7 | (2.3%) |
| targeted therapy | | | | | | |
| ICB + cytotoxic | 12 | (1.8%) | 10 | (2.7%) | 2 | (0.7%) |
| chemotherapy + | | | | | | |
| molecular | | | | | | |
| targeted therapy | | | | | | |
| ICB use* | | | | | | |
| Practice | 483 | (71.9%) | 261 | (70.2%) | | |
| Clinical trial | 161 | (24.0%) | 107 | (28.8%) | | |
| Unknown | 41 | (6.1%) | 17 | (4.6%) | | |
| Treatment setting* | | | | | | |
| Palliative | 557 | (82.9%) | 296 | 79.6%) | 261 | (87.0%) |
| Neoadjuvant | 43 | (6.4%) | 27 | (7.3%) | 16 | (5.3%) |
| Maintenance after | 32 | (4.8%) | 19 | (5.1%) | 13 | (4.3%) |
| definitive CRT | | | | | | |
| Adjuvant | 22 | (3.3%) | 13 | (3.5%) | 9 | (3.0%) |
| Neoadjuvant + | 21 | (3.1%) | 20 | (5.4%) | 1 | (0.3%) |
| adjuvant | | | | | | |
| Prior treatment** | | | | | | |
| Cytotoxic | 466 | (69.3%) | 252 | (67.7%) | 214 | (71.3%) |
| chemotherapy | | | | | | |
| Molecular | 129 | (19.2%) | 72 | (19.4%) | 57 | (19.0%) |
| targeted agent | | | | | | |
| RT | 163 | (24.3%) | 73 | (19.6%) | 90 | (30.0%) |
| CRT | 73 | (10.9%) | 51 | (13.7%) | 22 | (7.3%) |
| Number of prior | | | | | | |
| systemic therapy in | | | | | | |
| the palliative setting* | | | | | | |
| 0 | 70 | (10.4%) | 34 | (9.9%) | 36 | (12.0%) |
| 1 | 265 | (39.4%) | 138 | (37.1%) | 127 | (42.3%) |
| 2 | 113 | (16.8%) | 75 | (20.2%) | 38 | (12.7%) |
| >=3 | 87 | (12.9%) | 45 | (12.1%) | 42 | (14.0%) |

(*13 patients received two ICB therapies, and were calculated separately if applicable (when the basic features of the primary and secondary ICB therapies are different).
**Episodic and non-episodic prescription settings, such as adjuvant or neoadjuvant therapy, are included.

Immune-related adverse event (irAE); non-small cell lung cancer (NSCLC); hepatocellular carcinoma (HCC); cholangiocarcinoma (CCA); immune checkpoint blockade (ICB); radiation therapy (RT); chemical reaction treatment (CRT))

### Example 2. Classification of irAE Patients

For further analysis and training of integrated models, 84 irAE types (single label) were classified into 12 major labels (including label ‘Any’). The single label list consisting of 12 main labels is shown in Table 3 of the following Experimental Example 1, and the number of patients for each of the 12 main labels is shown in Table 4 of the following Experimental Example 1.

### Example 3. Clinical Features

Clinical features available for patients in the cohort of the present invention include a medication type, a cancer type, an ECOG performance status, the history of an autoimmune disease, and the history of diabetes or hypertension. Pre-treatment laboratory tests included several combination values calculated from values, such as a complete blood count (CBC), a chemical property, and a neutrophil-to-lymphocyte ratio (NLR), and also included a platelet-to-lymphocyte (PLR) ratio.

### Example 4. Whole Exome Sequencing (WES) and Data Processing

Libraries were prepared using a SureSelect Human All Exon V5 kit (Agilent Technologies, Santa Clara, CA), and clustering and sequencing were performed as follows according to the standard manufacturer's instructions using TruSeq Rapid SBS Kit-200 Cycle in HiSeq 2500 (Illumina, San Diego, CA).

The quality of raw WES FASTQ files was controlled using FastQC (v.0.11.9) and MultiQC (v.1.9). Reads were mapped to GRCh37 (hg19) build of the 1000 Genomes Project using BWA-MEM (v.0.7.17-r1188) along with default parameters. The mapped reads were classified using HTSlib (v.1.7-2) in SAMtools (v.1.7), and duplication was represented as Picard (v.2.25.0-5-ga2f44ae-SNAPSHOT). Remeasurement of the basic quality scores was done using ApplyBQSR of the Genome Analysis Toolkit (GATK; v.4.1.6.0).

### Example 5. RNA Sequencing and Data Processing

Libraries generated from a whole blood sample were sequenced as 150 bp paired-end reads on the Illumina platform. The quality of raw FASTQ files was controlled using FastQC (v.0.11.9), MultiQC (v.1.9), and Trimmomatic (v.0.39) (TruSeq3-PE-2.fa:2:30:10:2:keepBothReads LEADING:3 TRAILING:3 MINLEN:36 for adaptor sequencing trimming), and SortMeRNA (v.2.1b) (silva-euk-18s-id95.fasta, silva-euk-28s-id98.fasta) was used in rRNA filtering.

The reads were mapped against GRCh37 (hg19) build provided in the 1000 Genomes Project, and genes were assigned based on the gencode.v37.annotation.gtf using STAR 2 path mapping with sjdbOverhang 150. The mapped reads were classified using SAMtools (v.1.7), and the number of reads was counted by HTSeq (v.0.12.4). The number of reads was normalized by calculating TPM values using a house code.

### Example 6. Detection of Germline Single Nucleotide Variants (SNVs)

The GATK best practice workflow was adopted for SNV detection using analysis-ready bam files. After executing HaplotypeCaller in gVCF mode, GenomicsDBImport, GEnotypeGVCFs, VariantRecalibrator, and ApplyVQSR were performed. Variants that satisfied the criteria GQ>80 and DP>20 were filtered using VCFtools (v.0.1.15) and BCFtools (v.1.7), and information on intervals of exon regions was extracted with SureSelect Human All Exon V5 (Agilent).

### Example 7. Post-Processing of Merged Variant Call Format (VCF) Files

VCF files of 608 patients were merged and annotated with ANNOVAR. Only non-synonymous variants in which amino acid variations occurred were used for further analysis, and a binary code indicting the presence or absence of each variant in each patient was used for the p-value of the logistic regression for each irAE label, calculated using age, sex, and drug type as covariates. For variation in linkage disequilibrium, clumping was performed using the R package ieugwasr with parameters such as clumping window=250 kb, r²=0.8, P-value threshold=0.05, and East Asian population.

### Example 8. Detection of Germline Copy Number Variations (CNVs)

GATK germline CNVs were detected in cohort mode with parameters recommended by a best case. A read count was calculated using CollectReadCounts with the analysis-ready BAM files for each sample as input. Then, taking all the intervals as input, the interval lists processed using GATK PreprocessIntervals, AnnotateIntervals, and FilterIntervals were obtained. The copy number for each interval was calculated by DefineGermlineContigPloidy, GermlineCNVCaller, and PostprocessGermlineCNVCalls. Then, the CNV results of each patient were merged on an interval-by-interval basis by BCFtools merge function. The merged VCFs covered 224,551 intervals with a length of about 500 base pairs. To extract intervals (204,364 intervals) between exon regions, functional gene annotation was performed using ANNOVAR, and for samples not included in integrated model training, GATK germline CNV detection was performed in CASE mode. All exon intervals were classified into deletion, neutral, and duplication based on threshold copy number 2.

### Example 9. HLA-Typing

HLA genotyping was performed using HLA-HD (v.1.3.0) with WES FASTQ files as input resulting in typing both class I (HLA-A, B, and C) and class II (DRB1, DQB1, and DPB1). Up to four digits (i.e., the second field) were used for additional analysis, and binary coding was performed to confirm whether each patient has an allele corresponding to the HLA alleles pooled from all patients of the cohort of the present invention.

### Example 10. Multivariate Logistic Regression

Multivariate logistic regression using age, sex, and drug type as covariates was performed to identify a feature candidate associated with each irAE type. A control was defined as a patient who did not experience any irAE. Regressions were performed separately for binary codes of HLA-type and germline SNVs, and CNVs and the continuous value of a peripheral blood marker using age, sex, and drug type as covariates. The final significant feature was determined from the features with a regression p-value of 0.01 or less through a permutation test.

### Example 11. Generation of Training Input Features for Integrated Model

For each of the 12 main irAE labels, the tested germline variations were ranked based on multivariate logistic regression p-value. Among variations with P<0.01, up to 70 SNPs from the highest rank were used as training input features. The optimal number of variations for training was determined by finding the peaks in a plot, which shows the average precision of each test set as the number of trained variations increases, using the find_peaks( ) function in the SciPy package, and house code. In addition, it was intended to determine the optimal number of trained variations using XGBoost, which identified that the average precision was higher when the number of variations, determined by multivariate logistic regression, was used. The number of trained variations was limited to avoid overfitting the model.

### Example 12. Training and Prediction of Integrated Model

An integrated model was trained with each of the 12 main labels. The features of the integrated model include significantly associated HLA type, CNV and a peripheral blood marker, found by multivariate logistic regression, as well as the significantly associated germline variations selected by multivariate logistic regression, as described above. A deep neural network (DNN) framework was constructed to train the integrated model. The performance of DNN was superior to that of a XGBoost classifier. To train models for each label, irAE patients with the corresponding label (true case) and non-irAE patients (false case) were divided into training and validation sets (8:2). All features of the training set were scaled to the range between-1 to 1 using MinMaxScaler, and a scaler fitted to the training set was adopted for feature transformation of the validation set. The optimal model for predicting irAE occurrence, which is the main goal of the training, was selected based on an average precision value, and for model validation, a validation set and samples, which were not used in model training and validation, that is, all irAE patients who did not correspond to the given model label, were used.

The integrated model training was additionally performed with gene expression features for 250 patients for whom both WES data and RNA-seq data were available. The performance of models trained with features derived from WES data and RNA-seq data was compared using the average precision metric.

### Example 13. DNN Modeling

A DNN framework was implemented using PyTorch. For weight initialization, three fully-connected hidden layers and the Xavier uniform method were used. The 1^{st}, 2^{nd}, and 3^{rd} hidden layers have 40, 80, and 20 hidden nodes, respectively, tan h has an interlayer activation function, and sigmoid has a final activation function. All samples were divided into 5 batches to determine a batch size, and the Adam optimizer was applied for the optimization process, and the binary cross-entropy was applied for a loss function. The learning rate, maximum epoch, and patience for early interruption with the optimization process were set to 0.001, 100, and 5, respectively. All hyperparameters were determined by repeated sweeping, and the model with the lowest test loss within 100 epochs was selected as the final model.

### Example 14. Evaluation of Feature Importance

SHAP values for interpreting the impact of each feature on the prediction results were calculated using the DeepExplainer function of the SHAP package. The SHAP values of all samples for each variation were averaged to rank germline variations based on the SHAP values, and highly ranked variations were used for further analysis. The direct comparison between the SHAP values of variation features (binary code), and SHAP values of CNV (−1,0,1) and peripheral blood markers (continuous values) was not possible due to their different scale ranges.

### Example 15. Analysis of Cumulative Incidence

Cumulative incidence analysis was performed to investigate the correlation between time to irAE occurrence, (1) the copy number of gene HLA-B, and (2) the genotype of rs541169 variation. Patients with a copy number of 2 were classified as a normal polyploidy group, patients with a copy number of more than 2 were classified as a duplication group, and patients with a copy number of less than 2 were classified as a deletion group. Patients were classified into three groups according to their genotype for rs541169: homozygous reference allele (HomoRef), heterozygous alternative (HetAlt), and homozygous alternative (HomoAlt). The period from the start of ICB treatment to irAE occurrence was defined as a follow-up period, and death or follow-up loss was treated as censored data. Statistical significance was calculated using a Cox proportional hazards model.

### Example 16. Deconvolution of Cell Type Abundance from RNA-Seq Data

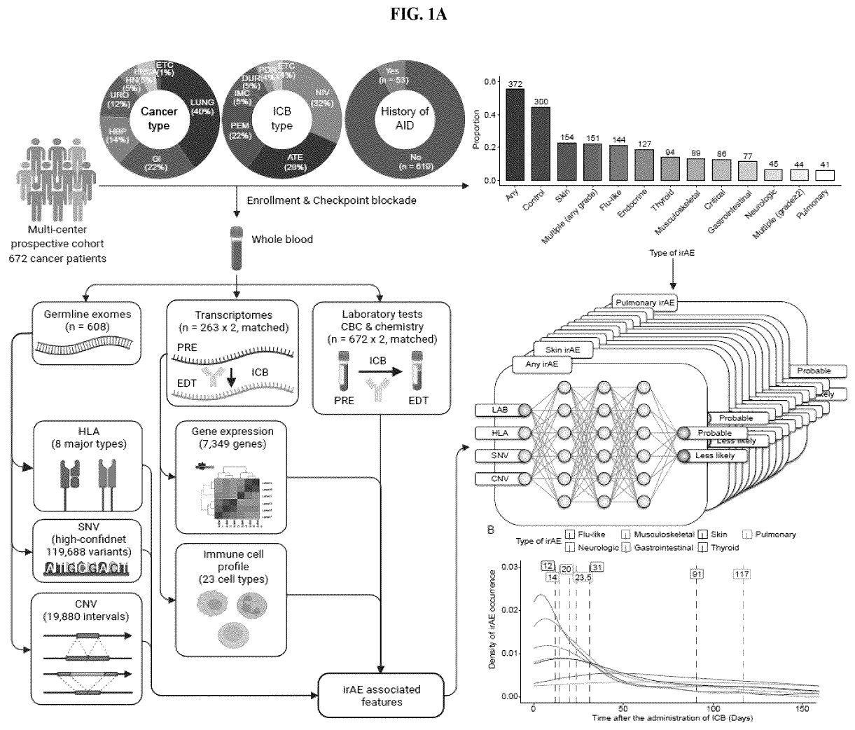

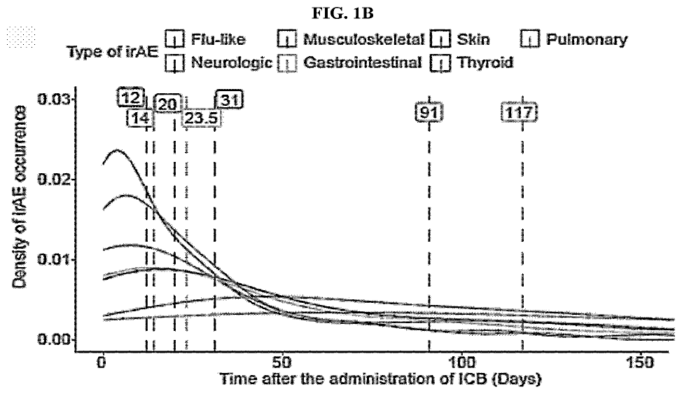

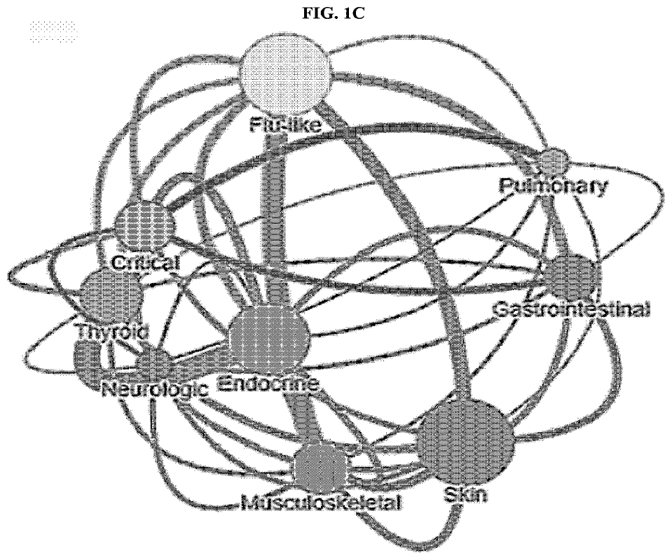

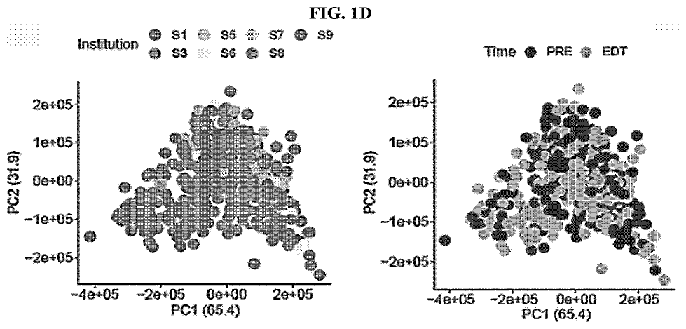

A total of 21 immune cell fractions from whole blood RNA-seq data were calculated using ImmuCellAI (Miao, Y et al., Adv. Sci. 7, 1902880.). PCA analysis was performed to minimize possible bias between sequencing data from different sequencing batches (refer to FIG. 1D), and PCA plots showed that the samples form a harmonious mixture in different sequencing batches.

### Example 17. Statistical Analysis for Balanced Signature Detection

To identify the signature of balancing selection, Hudson-Kreitman-Aguade (HKA) tests were performed on 26 subpopulations from the 1000 Genomes Project. HKA tests compare the level of polymorphism (diversity within a species) with the level of substitution (diversity between species. Maximum likelihood HKA tests (Wright and Charlesworth, 2004) were performed using MLHKA software (http://wright.eeb.utoronto.ca/programs/). The surrounding 1-kb region of the rs541169 mutation was compared with 99 neutrally evolved regions selected as previously reported (Fumagalli et al., 2009; Gokcumen et al., 2013). The number of segregating sites in each region and the pairwise number of differences between species were used as input, and chimpanzees were used as an outgroup in this analysis. To test selection, the program was run in a neutral model in which the number of selected loci was 0, and then in a selection model in which the surrounding 1-kb region of a focal SNP was considered as the only selected locus. Statistical significance was assessed by the likelihood ratio test in which twice the log likelihood difference between the selection model and the neutral model follows a x² distribution with a degree of freedom of 1 (the number of selected loci). To ensure the robustness of output, the chain length was set to 100,000. For each test site, the selection parameter k and the P-value were obtained from the likelihood ratio tests. The selection parameter k represents the k-fold increase in diversity relative to neutral expectations at a given locus. Therefore, k>1 supports balancing selection.

Abbreviations for subpopulations: AFR, African; AMR, Admixed American; EAS, East Asians; EUR, Europeans; SAS, South Asians; GWD, Gambian Mandinka; MSL, Mende, Sierra Leone; ASW, African ancestry in the Southwest, the US; ACB, African Caribbean, Barbados; YRI, Yoruba in Ibadan, Nigeria; LWK, Luhya of Webuye in Kenya; ESN, Esan in Nigeria; MXL, Mexican ancestry in Los Angeles, California; PUR, Puerto Ricans in Puerto Rico; CLM, Colombians in Medellin, Colombia; PEL, Peruvians in Lima, Peru; KHV, Kinh in Ho Chi Minh City, Vietnam; CDX, Chinese Dai of Xishuangbanna; CHB, Chinese Han in Beijing; KOR, Korean; CHS, Chinese Han in the South; JPT, Japanese in Tokyo; GBR, British in England and Scotland; IBS, Iberian Spanish; TSI, Tuscany people in Italy; CEU, Utah North and Western European ancestry (CEPH); FIN, Finnish, Finland; STU, Sri Lankan Tamils, United Kingdom; PJL, Punjabi in Lahore, Pakistan; GIH, Gujarati Indians in Houston, Texas; BEB, Bengali in Bangladesh; ITU, Indian Telugu, the UK.

### EXPERIMENTAL EXAMPLES

### Experimental Example 1. Multi-Center Pan-Cancer Prospective Cohort of irAEs

From 372 patients among 672 ICB-treated patients, 84 irAE types were identified (refer to the following Table 2). Based on affected organ systems, such as the skin, the endocrine system, the thyroid gland, the musculoskeletal system, the gastrointestinal system, and the neurologic system, a label was designated to each irAE. Depending on the severity of irAE, patients with 3 or more irAE types were represented as Multiple G>=1, patients with 3 or more grade 2 or higher irAE types were represented as Multiple G>=2, and patients with any grade 3 or higher irAE types and patients with grade 2 or higher and critical irAE types were additionally labeled as Critical. Other labels include Flu-like (flu-like symptoms) and Pulmonary (pneumonia due to ICB therapy), and patients under all irAE categories were represented as Any (refer to the following Tables 3 and 4).

TABLE 2

| | | irAE of | irAE of | |
|---|---|---|---|---|
| Type of irAE | Control | interest | non-interest | Proportion |
| Pruritus | 300 | 124 | 248 | 0.18 |
| Skin rash/ | 300 | 89 | 283 | 0.13 |
| dermatitis/urticaria | | | | |
| Myalgia | 300 | 72 | 300 | 0.11 |
| Hypothyroidism | 300 | 69 | 303 | 0.10 |
| Fatigue/asthenia | 300 | 62 | 310 | 0.09 |
| Subclinical | 300 | 48 | 324 | 0.07 |
| hypothyroidism | | | | |
| Pneumonitis | 300 | 43 | 329 | 0.06 |
| Hyperthyroidism | 300 | 25 | 347 | 0.04 |
| Hepatitis | 300 | 23 | 349 | 0.03 |
| Enterocolitis/diarrhea | 300 | 22 | 350 | 0.03 |
| Fever | 300 | 16 | 356 | 0.02 |
| Headache | 300 | 15 | 357 | 0.02 |
| Polyneuropathy/ | 300 | 14 | 358 | 0.02 |
| peripheral sensory | | | | |
| neuropathy | | | | |
| Anorexia | 300 | 13 | 359 | 0.02 |
| Arthralgia | 300 | 12 | 360 | 0.02 |
| Dry mouth | 300 | 11 | 361 | 0.02 |
| Adrenal insufficiency | 300 | 11 | 361 | 0.02 |
| Asymptomatic | 300 | 10 | 362 | 0.01 |
| lipase elevation | | | | |
| Asymptomatic | 300 | 10 | 362 | 0.01 |
| amylase elevation | | | | |
| Dizziness | 300 | 10 | 362 | 0.01 |
| Infusion-related | 300 | 10 | 362 | 0.01 |
| reaction | | | | |
| Subclinical | 300 | 9 | 363 | 0.01 |
| hyperthyroidism | | | | |
| Stomatitis | 300 | 8 | 364 | 0.01 |
| Nausea | 300 | 8 | 364 | 0.01 |
| Sweating | 300 | 7 | 365 | 0.01 |
| Muscle weakness | 300 | 7 | 365 | 0.01 |
| Creatine | 300 | 6 | 366 | 0.01 |
| phosphokinase | | | | |
| elevation | | | | |
| Hoarseness | 300 | 6 | 366 | 0.01 |
| Vomiting | 300 | 5 | 367 | 0.01 |
| Dysesthesia | 300 | 5 | 367 | 0.01 |
| Type I diabetes | 300 | 5 | 367 | 0.01 |
| mellitus | | | | |
| Serum creatinine | 300 | 4 | 368 | 0.01 |
| elevation | | | | |
| Skin hypopigmentation | 300 | 3 | 369 | 0.00 |
| Proteinuria | 300 | 3 | 369 | 0.00 |
| Lichen planus | 300 | 3 | 369 | 0.00 |
| Gastritis | 300 | 3 | 369 | 0.00 |
| Limb edema | 300 | 3 | 369 | 0.00 |
| Abdominal pain | 300 | 3 | 369 | 0.00 |
| Panhypopituitarism/ | 300 | 2 | 370 | 0.00 |
| hypophysitis | | | | |
| Uveitis | 300 | 2 | 370 | 0.00 |
| Sore throat | 300 | 2 | 370 | 0.00 |
| Pericarditis | 300 | 2 | 370 | 0.00 |
| Pancreatitis | 300 | 2 | 370 | 0.00 |
| Nephritis | 300 | 2 | 370 | 0.00 |
| Myositis | 300 | 2 | 370 | 0.00 |
| Insomnia | 300 | 2 | 370 | 0.00 |
| Hypertension | 300 | 2 | 370 | 0.00 |
| Facial edema | 300 | 2 | 370 | 0.00 |
| Dry eye | 300 | 2 | 370 | 0.00 |
| Conjunctivitis | 300 | 2 | 370 | 0.00 |
| Retinopathy | 300 | 1 | 371 | 0.00 |
| Blurred vision | 300 | 1 | 371 | 0.00 |
| Eye pain | 300 | 1 | 371 | 0.00 |
| Epiphora | 300 | 1 | 371 | 0.00 |
| Meningoencephalitis | 300 | 1 | 370 | 0.00 |
| Meningitis | 300 | 1 | 370 | 0.00 |
| Leukoencephalopathy | 300 | 1 | 371 | 0.00 |
| Cognitive dysfunction | 300 | 1 | 371 | 0.00 |
| Delirium | 300 | 1 | 371 | 0.00 |
| Trigeminal neuralgia | 300 | 1 | 371 | 0.00 |
| Myasthenia gravis | 300 | 1 | 371 | 0.00 |
| Myopathy | 300 | 1 | 371 | 0.00 |
| Myocarditis | 300 | 1 | 371 | 0.00 |
| Renal thrombotic | 300 | 1 | 371 | 0.00 |
| microangiopathy | | | | |
| Hemolytic | 300 | 1 | 371 | 0.00 |
| uremic syndrome | | | | |
| Pulmonary embolism | 300 | 1 | 371 | 0.00 |
| Hematuria | 300 | 1 | 371 | 0.00 |
| Tympanitis | 300 | 1 | 371 | 0.00 |
| Tinnitus | 300 | 1 | 371 | 0.00 |
| Tongue pain | 300 | 1 | 371 | 0.00 |
| Hyposmia | 300 | 1 | 371 | 0.00 |
| Bullous pemphigoid | 300 | 1 | 371 | 0.00 |
| Dry skin | 300 | 1 | 371 | 0.00 |
| Skin | 300 | 1 | 371 | 0.00 |
| hyperpigmentation | | | | |
| Paronychia | 300 | 1 | 371 | 0.00 |
| Herpes zoster | 300 | 1 | 371 | 0.00 |
| Hand foot syndrome | 300 | 1 | 371 | 0.00 |
| Constipation | 300 | 1 | 371 | 0.00 |
| Interstitial fibrosis | 300 | 1 | 371 | 0.00 |
| Exacerbation | 300 | 1 | 371 | 0.00 |
| of COPD | | | | |
| Thrombocytopenia | 300 | 1 | 371 | 0.00 |
| Neutropenia | 300 | 1 | 371 | 0.00 |
| Exacerbation of | 300 | 1 | 371 | 0.00 |
| rheumatoid arthritis | | | | |
| Allergic rhinitis | 300 | 1 | 371 | 0.00 |

TABLE 3

| Type of irAE | Description | Components |
|---|---|---|
| Any | Any kinds of irAE | single labels |
| Critical | Any irAEs of grade ≥3 or | *adrenal insufficiency, hypophysitis, panhypopituitarism, |
| | critical irAEs* of grade ≥2 | type I diabetes mellitus, pancreatitis, |
| | | leukoencephalopathy, meningoencephalitis, meningitis, |
| | | pericarditis, myocarditis, nephritis, renal thrombotic |
| | | microangiopathy, hemolytic uremic syndrome, pulmonary |
| | | embolism, pneumonitis |
| Skin | irAEs related with skin | skin hyperpigmentation |
| | | skin hypopigmentation |
| | | hand foot syndrome |
| | | dry skin |
| | | dermatitis |
| | | skin rash |
| | | paronychia |
| | | lichen planus |
| | | pruritus |
| | | urticaria |
| | | bullous pemphigoid |
| Thyroid | irAEs related with | hyperthyroidism |
| | thyroid gland | hypothyroidism |
| | | subclinical hypothyroidism with TSH >10 μU/mL |
| Endocrine | irAEs related with endocrine | hyperthyroidism |
| | system | hypothyroidism |
| | | subclinical hyperthyroidism |
| | | subclinical hypothyroidism |
| | | subclinical hypothyroidism with TSH >10 μU/mL |
| | | adrenal insufficiency |
| | | panhypopituitarism |
| | | hypophysitis |
| | | type I diabetes mellitus |
| Musculoskeletal | irAEs related with | arthralgia |
| | musculoskeletal system | myalgia |
| | | myositis |
| | | myopathy |
| | | muscle weakness |
| | | exacerbation of rheumatoid arthritis |
| Neurologic | irAEs related with neurologic | leukoencephalopathy |
| | system | meningoencephalitis |
| | | meningitis |
| | | headache |
| | | dizziness |
| | | cognitive dysfunction |
| | | delirium |
| | | Peripheral sensory neuropathy |
| | | polyneuropathy |
| | | dysesthesia |
| | | trigeminal neuralgia |
| | | myasthenia gravis |
| Pulmonary | Pneumonitis of any grade | pneumonitis |
| Gastrointestinal | irAEs related with | hepatitis |
| | gastrointestinal system | pancreatitis |
| | | asymptomatic amylase/lipase elevation |
| | | enterocolitis |
| | | diarrhea |
| | | gastritis |
| | | nausea |
| | | vomiting |
| | | anorexia |
| | | abdominal pain |
| | | constipation |
| Flu-like | Flu-like symptoms | fever |
| | | sweating |
| | | fatigue |
| | | arthralgia |
| | | muscle weakness |
| | | myalgia |
| | | sore throat |
| | | headache |
| Multiple | Patients with 3 or more | single labels |
| (any grade) | irAEs of any grade | |
| Multiple | Patients with 3 or more | single labels |
| (grade ≥2) | irAEs of grade ≥2 | |

The clinical features of 12 labeled irAE patient groups (irAE groups) and non-irAE patient group (control) are shown in Table 4 below.

TABLE 4

| | Type of irAE | | | | | | | | |
|---|---|---|---|---|---|---|---|---|---|
| out of 672 patients | Any | | Critical | | Skin | | Thyroid | | Ctrl |
| Number of patients (%) | 372 | (100%) | 90 | (23%) | 156 | (41%) | 96 | (25%) | 300 |

| | | | | | |
|---|---|---|---|---|---|
| Age, median (years) | 63 | 62 | 62 | 60 | 64 |

| | | | | | | | | | | |
|---|---|---|---|---|---|---|---|---|---|---|
| Male (%) | 275 | (74%) | 66 | (73%) | 120 | (77%) | 59 | (61%) | 215 | (72%) |
| ECOG Performance status | | | | | | | | | | |
| 0-1 | 328 | (89%) | 75 | (87%) | 133 | (86%) | 85 | (89%) | 259 | (87%) |
| ≥2 | 41 | (11%) | 11 | (13%) | 21 | (14%) | 10 | (11%) | 40 | (13%) |
| NSCLC (%) | 134 | (36%) | 43 | (48%) | 54 | (35%) | 27 | (28%) | 123 | (41%) |
| Prior treatment | | | | | | | | | | |
| Cytotoxic | 252 | (68%) | 49 | (54%) | 98 | (63%) | 45 | (47%) | 214 | (71%) |
| chemotherapy | | | | | | | | | | |
| Molecular targeted | 72 | (19%) | 16 | (18%) | 28 | (18%) | 14 | (15%) | 57 | (19%) |
| agent | | | | | | | | | | |
| RT | 73 | (20%) | 18 | (20%) | 28 | (18%) | 18 | (19%) | 90 | (30%) |
| CRT | 51 | (13%) | 9 | (10%) | 22 | (14%) | 10 | (10%) | 22 | (7%) |
| Median of prior | 1 | (0-7) | 1 | (0-5) | 1 | (0-7) | 1 | (0-3) | 1 | (0-6) |
| systemic therapy in | | | | | | | | | | |
| the palliative setting | | | | | | | | | | |
| (range) | | | | | | | | | | |
| Comorbidity | | | | | | | | | | |
| HTN | 125 | (34%) | 28 | (31%) | 57 | (37%) | 28 | (29%) | 101 | (34%) |
| DM | 63 | (17%) | 20 | (22%) | 28 | (18%) | 19 | (20%) | 51 | (17%) |
| History of | 33 | (9%) | 9 | (10%) | 11 | (7%) | 13 | (14%) | 20 | (7%) |
| autoimmune disease | | | | | | | | | | |
| anti-PD-1 ICB (%) | 233 | (61%) | 58 | (64%) | 99 | (63%) | 57 | (59%) | 167 | (56%) |

| | Type of irAE | | | | | | | | | |
|---|---|---|---|---|---|---|---|---|---|---|
| out of 672 patients | Endocrine | | Musculoskeletal | | Neurologic | | Pulmonary | | Ctrl | |
| Number of patients (%) | 130 | (34%) | 90 | (23%) | 49 | (13%) | 43 | (11%) | 300 | |
| Age, median (years) | 61 | | 61 | | 61 | | 63 | | 64 | |
| Male (%) | 87 | (67%) | 65 | (72%) | 39 | (80%) | 33 | (77%) | 215 | (72%) |
| ECOG Performance status | | | | | | | | | | |
| 0-1 | 118 | (91%) | 76 | (84%) | 43 | (88%) | 38 | (93%) | 259 | (87%) |
| ≥2 | 11 | (9%) | 14 | (16%) | 6 | (12%) | 3 | (7%) | 40 | (13%) |
| NSCLC (%) | 37 | (28%) | 34 | (38%) | 17 | (35%) | 27 | (63%) | 123 | (41%) |
| Prior treatment | | | | | | | | | | |
| Cytotoxic | 69 | (53%) | 60 | (67%) | 29 | (59%) | 25 | (58%) | 214 | (71%) |
| chemotherapy | | | | | | | | | | |
| Molecular targeted | 17 | (13%) | 15 | (17%) | 10 | (20%) | 6 | (14%) | 57 | (19%) |
| agent | | | | | | | | | | |
| RT | 22 | (17%) | 10 | (11%) | 4 | (8%) | 10 | (23%) | 90 | (30%) |
| CRT | 16 | (12%) | 17 | (19%) | 9 | (18%) | 8 | (19%) | 22 | (7%) |
| Median of prior | 1 | (0-3) | 1 | (0-5) | 1 | (0-4) | 1 | (0-5) | 1 | (0-6) |
| systemic therapy in | | | | | | | | | | |
| the palliative setting | | | | | | | | | | |
| (range) | | | | | | | | | | |
| Comorbidity | | | | | | | | | | |
| HTN | 43 | (33%) | 34 | (38%) | 19 | (39%) | 13 | (30%) | 101 | (34%) |
| DM | 28 | (22%) | 11 | (12%) | 8 | (16%) | 9 | (21%) | 51 | (17%) |
| History of | 16 | (12%) | 8 | (9%) | 4 | (8%) | 4 | (9%) | 20 | (7%) |
| autoimmune disease | | | | | | | | | | |
| anti-PD-1 ICB (%) | 76 | (58%) | 54 | (60%) | 30 | (61%) | 29 | (67%) | 167 | (56%) |

| | Type of irAE | | | | | | | | | |
|---|---|---|---|---|---|---|---|---|---|---|
| out of 672 patients | Gastrointestinal | | Flu-like | | Multiple (any grade) | | Multiple (grade ≥2) | | Ctrl | |
| Number of patients (%) | 78 | (20%) | 148 | (38%) | 155 | (40%) | 45 | (12%) | 300 | |
| Age, median (years) | 63 | | 62 | | 62 | | 62 | | 64 | |
| Male (%) | 61 | (78%) | 105 | (71%) | 117 | (75%) | 37 | (82%) | 215 | (72%) |
| ECOG Performance status | | | | | | | | | | |
| 0-1 | 43 | (88%) | 132 | (89%) | 136 | (89%) | 35 | (81%) | 259 | (87%) |
| ≥2 | 6 | (12%) | 16 | (11%) | 16 | (11%) | 8 | (19%) | 40 | (13%) |
| NSCLC (%) | 27 | (35%) | 54 | (36%) | 52 | (34%) | 14 | (31%) | 123 | (41%) |
| Prior treatment | | | | | | | | | | |
| Cytotoxic | 44 | (56%) | 95 | (64%) | 90 | (58%) | 24 | (53%) | 214 | (71%) |
| chemotherapy | | | | | | | | | | |
| Molecular targeted | 13 | (17%) | 24 | (16%) | 28 | (18%) | 8 | (18%) | 57 | (19%) |
| agent | | | | | | | | | | |
| RT | 11 | (14%) | 22 | (15%) | 21 | (14%) | 5 | (11%) | 90 | (30%) |
| CRT | 9 | (12%) | 20 | (14%) | 18 | (12%) | 6 | (13%) | 22 | (7%) |
| Median of prior | 1 | (0-4) | 1 | (0-6) | 1 | (0-4) | 1 | (0-4) | 1 | (0-6) |
| systemic therapy in | | | | | | | | | | |
| the palliative setting | | | | | | | | | | |
| (range) | | | | | | | | | | |
| Comorbidity | | | | | | | | | | |
| HTN | 28 | (36%) | 54 | (36%) | 57 | (37%) | 15 | (33%) | 101 | (34%) |
| DM | 14 | (18%) | 22 | (15%) | 32 | (21%) | 11 | (24%) | 51 | (17%) |
| History of | 6 | (8%) | 12 | (8%) | 12 | (8%) | 2 | (4%) | 20 | (7%) |
| autoimmune disease | | | | | | | | | | |
| anti-PD-1 ICB (%) | 44 | (56%) | 86 | (58%) | 97 | (63%) | 28 | (62%) | 167 | (56%) |

According to Table 4, lung cancer was the most common cancer type, and patients with lung cancer were mostly treated with anti-PD-1. There was no significant difference between the Eastern Cooperative Oncology Group performance status (ECOG PS) and the history of autoimmune diseases in all irAE groups. The ICB agents used in this cohort included 5 types of PD-1 antibodies (pembrolizumab: PEM, nivolumab: NIV, PDR001: PDR, INCMGA00012: INC, tislelizumab: TIS), 4 types of PD-L1 antibodies (atezolizumab: ATE, durvalumab: DUR, IMC-001: IMC, avelumab: AVE), 2 types of CTLA-4 antibodies (ipilimumab: IPI, tremelimumab: TRE) used in combination with PD-1 or PD-L1 antibody, PD-1 expressing T cells preferentially targeting CTLA-4 (MEDI5752), and a bispecific antibody against STING agonist (MK1454) and ILT4 antibody (MK4830).

According to the following Table 5, while 55% of all cases were labeled as Any, Skin was the most frequent irAE type (23%), followed by Multiple (all grades; 23%) and Flu-like (22%) (refer to FIG. 1A).

TABLE 5

| | | irAE of | irAE of | |
|---|---|---|---|---|
| Type of irAE | Control | interest | non-interest | Proportion |
| Any | 300 | 372 | | 0.55 |
| Skin | 300 | 156 | 229 | 0.23 |
| Multiple (any grade) | 300 | 155 | 230 | 0.23 |
| Flu-like | 300 | 148 | 237 | 0.22 |
| Endocrine | 300 | 130 | 255 | 0.19 |
| Thyroid | 300 | 96 | 289 | 0.14 |
| Musculoskeletal | 300 | 90 | 295 | 0.13 |
| Critical | 300 | 90 | 295 | 0.13 |
| Gastrointestinal | 300 | 78 | 307 | 0.12 |
| Neurologic | 300 | 49 | 336 | 0.07 |
| Multiple (grade ≥2) | 300 | 45 | 340 | 0.07 |
| Pulmonary | 300 | 43 | 342 | 0.06 |

In addition, as shown in FIG. 1B, the Flu-like case showed the earliest occurrence (median=12 days), and Pulmonary showed the latest occurrence (median=117 days), followed by Thyroid (median=91 days). In FIG. 1B, each vertical dashed line and number represents the median of the occurrence data for each irAE type. As shown in FIG. 1C, network analysis showed strong co-occurrence between Thyroid and Endocrine, Skin, Flu-like, and Musculoskeletal.

Moreover, to identify genetic, molecular, and cellular irAE risk factors, multi-dimensional sequencing was performed on this cohort. Germline variations were screened based on whole exome sequencing of baseline whole blood samples obtained from 608 patients prior to ICB treatment (PRE), and SNV, CNV, and HLA typing were included in the analysis. RNA sequencing was performed on 263 matched whole blood samples before ICB treatment (PRE) and in early ICB treatment (EDT) to investigate differential molecular activity and immune cell profiles between patients with or without irAE and between PRE and EDT. Table 6 below shows the number of available samples according to clinical factors. CBC tests and biological analyses were performed on all PRE and EDT samples to investigate not only the baseline differences but also changes caused by ICB treatment between the irAE groups and the non-irAE group (refer to FIG. 1A).

TABLE 6

| Sex | | 672 | |
|---|---|---|---|
| Age | | 672 | |
| Cancer type | | 672 | |
| AID | | 672 | |
| HTN | | 672 | |
| DM | | 672 | |
| ECOG | | 668 | |
| cytotoxic agent | | 626 | |
| molecular target agent | | 625 | |
| RT | | 635 | |
| CCRT | | 635 | |
| PRE WBC | 672 | EDT WBC | 631 |
| PRE RBC | 669 | EDT RBC | 630 |
| PRE Hemoglobin | 672 | EDT Hemoglobin | 629 |
| PRE Hematocrit | 672 | EDT Hematocrit | 631 |
| PRE Platelet | 672 | EDT Platelet | 631 |
| PRE Lymphocyte | 669 | EDT Lymphocyte | 630 |
| PRE Neutrophil | 669 | EDT Neutrophil | 630 |
| PRE Monocyte | 669 | EDT Monocyte | 630 |
| PRE Eosinophil | 660 | EDT Eosinophil | 630 |
| PRE Basophil | 665 | EDT Basophil | 630 |
| PRE ANC | 670 | EDT ANC | 612 |
| PRE Abs Neutrophil | 669 | EDT Abs Neutrophil | 629 |
| PRE Abs Lymphocyte | 669 | EDT Abs Lymphocyte | 629 |
| PRE NLR | 669 | EDT NLR | 629 |
| PRE PLR | 669 | EDT PLR | 629 |
| PRE Calcium | 655 | EDT Calcium | 620 |
| PRE Phosphorus | 424 | EDT Phosphorus | 411 |
| PRE Glucose | 644 | EDT Glucose | 613 |
| PRE BUN | 491 | EDT BUN | 466 |
| PRE UA | 595 | EDT UA | 571 |
| PRE Cholesterol | 346 | EDT Cholesterol | 315 |
| PRE Protein | 656 | EDT Protein | 611 |
| PRE Albumin | 665 | EDT Albumin | 625 |
| PRE AST | 671 | EDT AST | 630 |
| PRE ALT | 669 | EDT ALT | 628 |
| PRE ALP | 652 | EDT ALP | 616 |
| PRE gGT | 142 | EDT gGT | 123 |
| PRE LDH | 345 | EDT LDH | 331 |
| PRE Bilirubin | 668 | EDT Bilirubin | 629 |
| PRE Amylase | 148 | EDT Amylase | 189 |
| PRE Lipase | 134 | EDT Lipase | 176 |
| PRE CRP | 259 | EDT CRP | 246 |
| PRE Sodium | 540 | EDT Sodium | 529 |
| PRE Potassium | 541 | EDT Potassium | 530 |
| PRE Chloride | 538 | EDT Chloride | 528 |
| PRE Creatinine | 668 | EDT Creatinine | 630 |

### Experimental Example 2. Confirmation of Decreased Neutrophile Function in all PRE and EDT irAE Samples

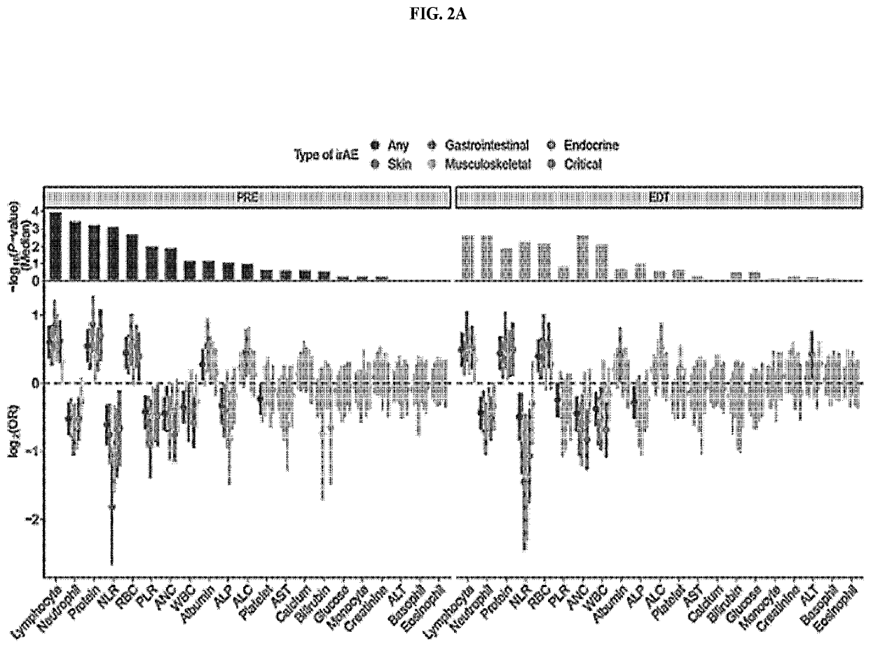

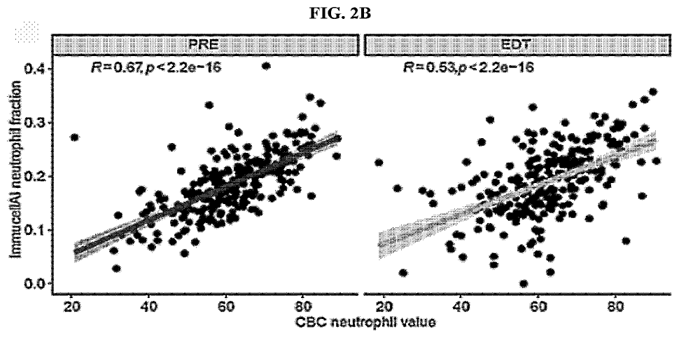

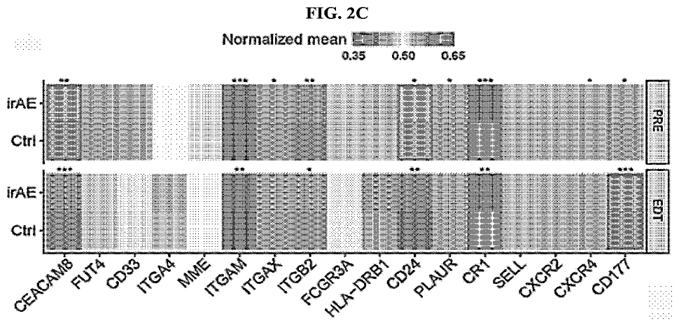

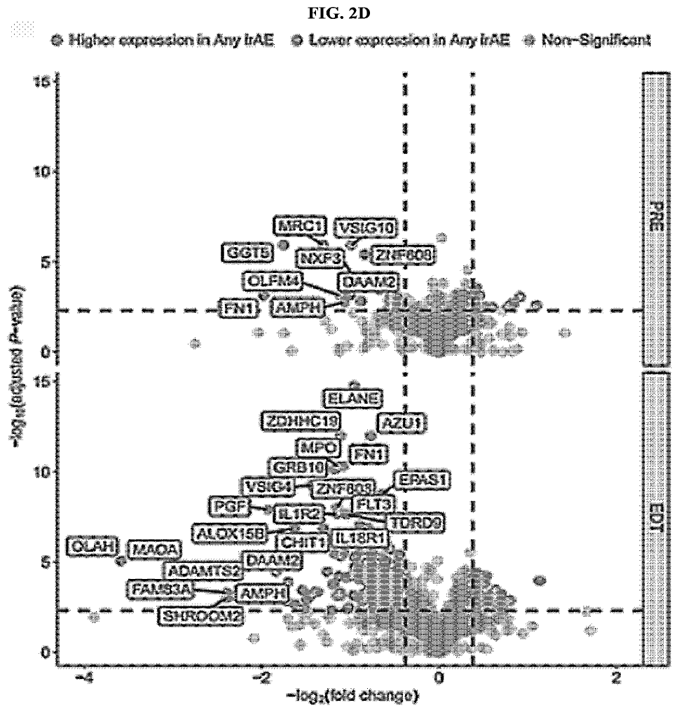

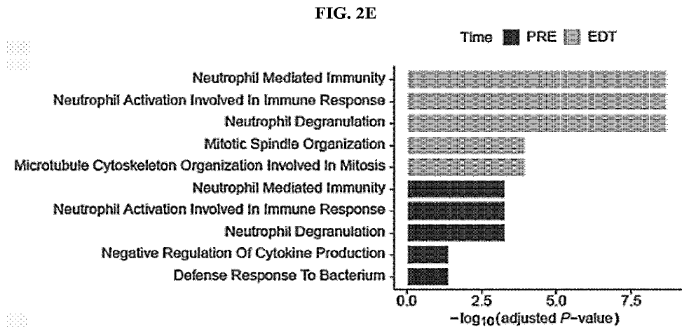

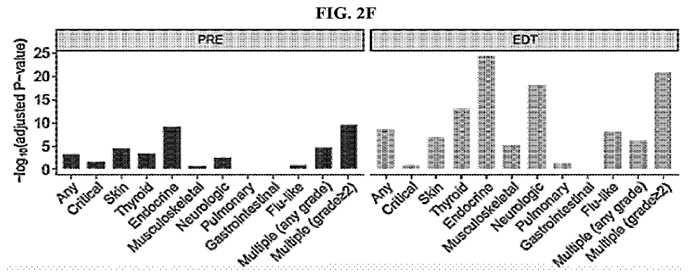

The association between irAE occurrence and CBC or biochemical measurement was examined using a generalized linear model including age and sex as covariates. As a result, as shown in FIG. 2A, the neutrophil counts, lymphocyte counts, and neutrophil-to-lymphocyte ratios (NLRs) of all PRE and EDT samples were associated with irAE occurrence in most irAE labels except Critical.

Specifically, all irAE risks were related to a significantly lower neutrophil count (PRE: odds ratio, 95% CI=0.69 (0.63-0.75), P=7.7e-06; EDT: odds ratio, 95% CI=0.73 (0.67-0.80), P=0.0002) and NLR (PRE: odds ratio, 95% CI=0.65 (0.59-0.72), P=2.36e-05, EDT: odds ratio, 95% CI=0.70 (0.62-0.80), P=0.004) as well as a higher lymphocyte count (PRE: odds ratio, 95% CI=1.52 (1.40-1.65), P=4e-07, EDT: odds ratio, 95% CI=1.40 (1.29-1.53), P=6.37e-05). The lower baseline NLR of irAE is consistent with the previous reports (Matsukane, R et al., Sci. Rep. 11, 1324.; Michailidou, D et al., Sci. Rep. 11, 9029; Pavan, A et al., Oncologist 24, 1128-1136).

In addition, white blood cell (WBC) and red blood cell (RBC) levels in all PRE and EDT samples were also associated with many irAE labels. However, irAE occurrence was only related to a platelet count, PLR, a protein level, an albumin level, and an alkaline phosphatase (ALP) concentration in the PRE samples. In contrast, the association of irAEs with a calcium level and an alanine aminotransferase (ALT) concentration was observed only in the EDT samples.

As a result of measuring a neutrophil cell fraction based on the inference from RNA sequencing data, as shown in FIG. 2B, there was a strong correlation between the CBC neutrophil count and the estimated neutrophil fraction. Likewise, the expression level of a neutrophil marker gene was significantly lower in the irAE group than a control in all PRE and EDT samples as shown in FIG. 2C.

Afterward, irAE-related differences were numerically characterized at the molecular level besides the cell count. To this end, genes with higher or lower expression in irAE samples were confirmed. As a result, as shown in FIG. 2D, particularly, differential gene expression was mostly characterized by low expression in the irAE groups, which was more obvious in the EDT samples than the PRE samples. As shown in FIG. 2E, pathway enrichment analysis showed that irAE-related gene repression occurred in neutrophil-mediated immunity as well as neutrophil activation and degranulation in all PRE and EDT samples. However, the degree of pathway enrichment was much greater in the case of genes identified in the EDT samples. When confirmed for each of 12 irAE groups, as shown in FIG. 2F, a similar pattern was observed when low-expressed genes were identified from the comparison between each of the 12 irAE groups and the control.

### Experimental Example 3. Confirmation of Activation of Differential Treatment Genes Between irAE Patients and Non-irAE Patients

The present inventors sought to understand how gene expression programs respond differently to ICB treatment between the irAE groups and the control by comparing the matched PRE and EDT samples. In the case of the patents with irAEs, genes that were significantly up-regulated in ICB treatment included genes that are responsible for cytolytic activity (IFNG, GZMH, GZMA) and NK cell activation (CD160, NKG7) (Chen, I. X et al., Proc Natl Acad Sci USA. 2020 Sep. 22; 117 (38): 23684-23694.). In addition, immunosuppressive genes, such as IDO1, which can promote the function of regulatory T lymphocytes, were also included (Hornyak, L et al., Front. Immunol. 9, 151.). This can reflect a homeostatic control mechanism acting by antitumor immune activation after ICB treatment.

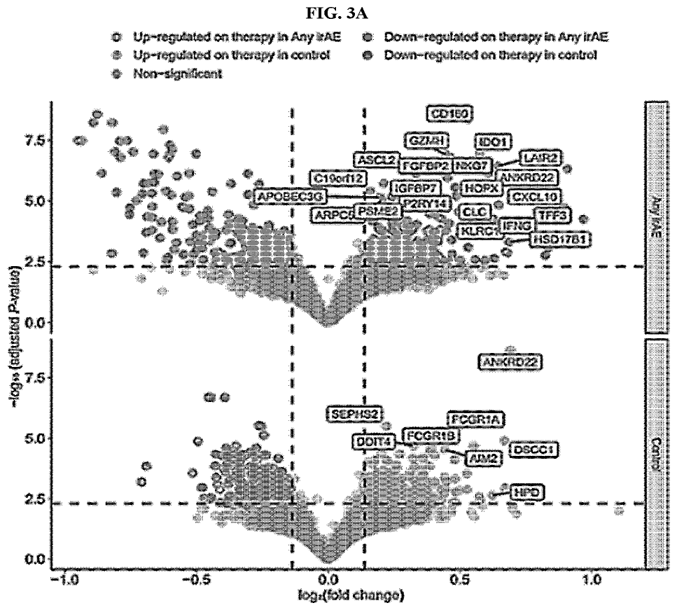

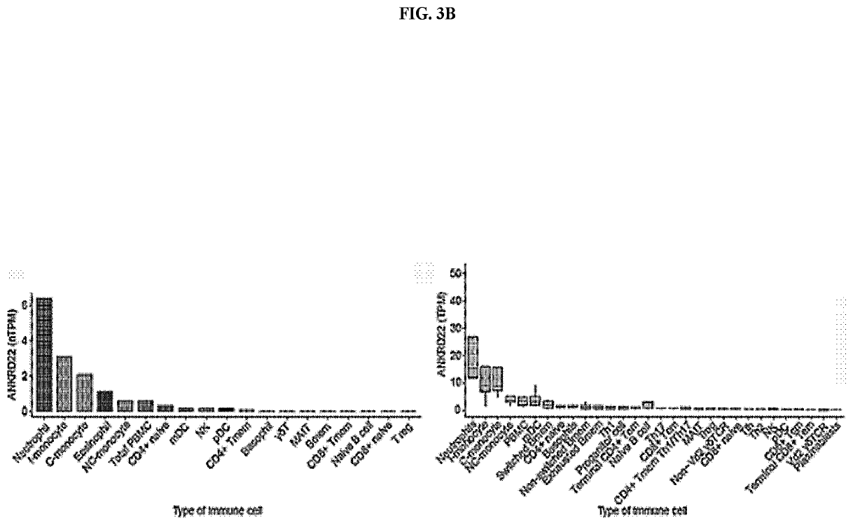

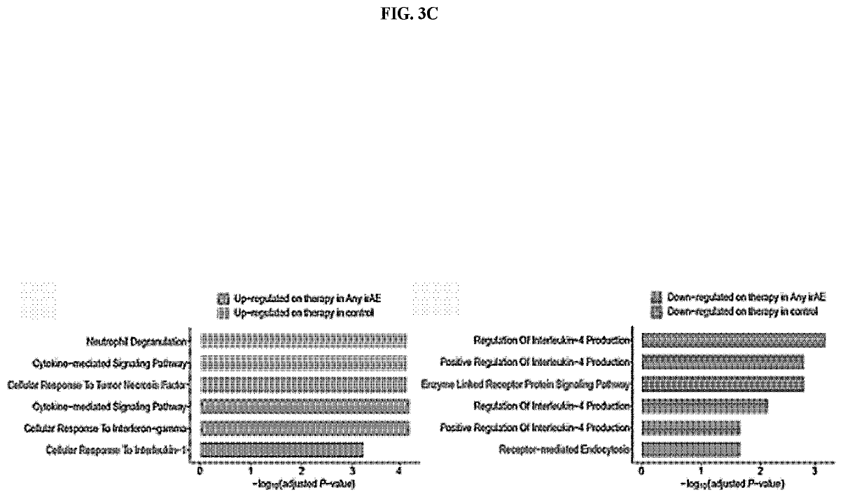

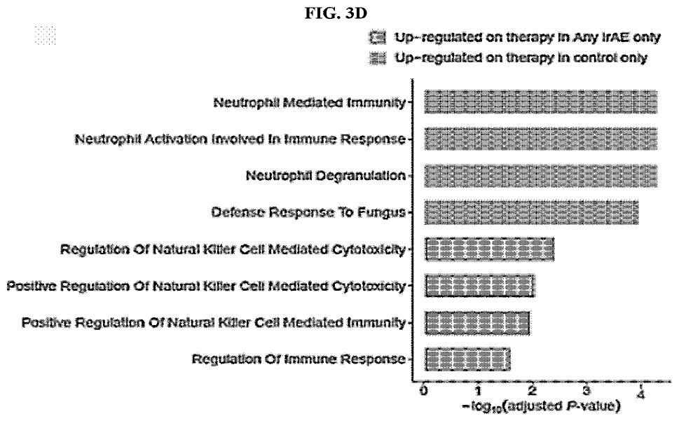

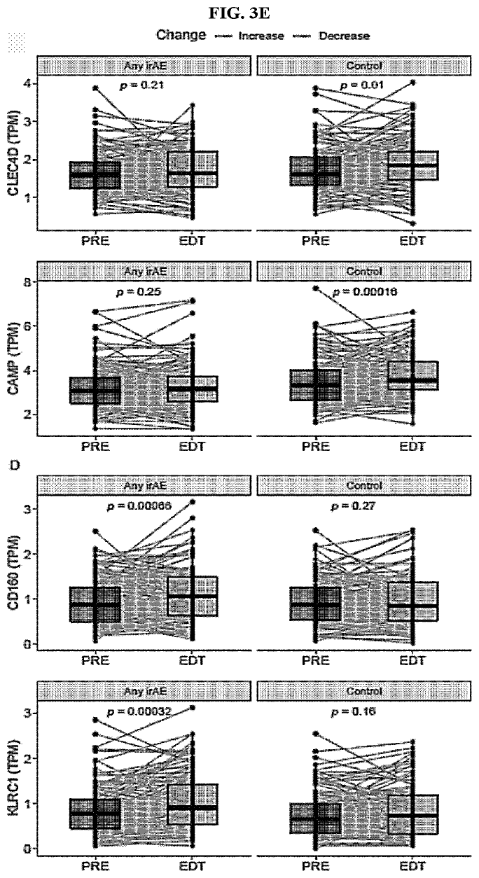

As shown in FIG. 3A, ANKRD22 is one of the genes significantly up-regulated in the EDT samples of the control. Although its function is not well established, the induction of the gene in peripheral blood has been observed in host defense against viral infection (Bin, L et al., J Immunol (2016) 196 (1_Supplement): 201.4.). In the present invention, it was found that, as shown in FIG. 3B, ANKRD22 is specifically expressed in neutrophils.

As shown in FIG. 3C, the pathway enrichment analysis mainly showed up-regulation of EDT for immune activation, such as cytokine-mediated signaling and cellular responses to tumor necrosis factors and IFN-γ in all irAE groups and control. In addition to these pathways, the EDT samples of the control showed the activation of neutrophil-related functions. Pathway enrichment was further investigated for genes that were exclusively activated only in the irAE groups or the control, and genes involved in cytokine-mediated signaling and cellular responses were excluded. As a result, as shown in FIG. 3D, it was confirmed that the NK cell-related gene expression program and the neutrophil-related gene expression program were specifically activated in the irAE groups and the control, respectively. For example, as shown in FIG. 3E, genes associated with neutrophil activity, such as CLEC4D and CAMP, were activated upon treatment only in the control. On the other hand, some genes associated with NK cell activity, such as CD160 and KLRC1, were up-regulated to the response to treatment, especially in the irAE patients.

### Experimental Example 4. Confirmation of HLA-B Alleles and CNV Associated with Overall irAE Risk

Multivariate logistic regression was performed using age, sex, and a drug type as covariates to evaluate the association between a copy number in 19,880 exon interval units and the occurrence of 12 major irAEs types. Significantly associated exon intervals (P<0.01) are shown in Table 7 below. Particularly, it was revealed that the CNV of HLA Class I and II genes was significantly associated with various irAE types.

TABLE 7

| Type of irAE | CNV ID | P-value | gene |
|---|---|---|---|
| Any | chr6: 31324462-31324741 | 0.001 | HLA-B wholegene |
| | | | HLA-B exon2 |
| Any | chr7: 100634945-100635138 | 0.002 | MUC12 exon2 |
| Any | chr2: 87420635-87420975 | 0.002 | ANAPC1 exon8 |
| Any | chr8: 54793570-54793940 | 0.003 | RGS20 wholegene |
| Any | chr1: 152586094-152586493 | 0.004 | LCE3B wholegene |
| Any | chr22: 40081915-40082662 | 0.005 | CACNA1I exon36 |
| | | | CACNA1I exon37 |
| Any | chr1: 35370310-35370516 | 0.007 | DLGAP3 exon1 |
| Any | chr17: 77812551-77812932 | 0.007 | CBX4 exon2 |
| Any | chr4: 69342831-69343615 | 0.008 | TMPRSS11E exon8 |
| Any | chr1: 152573024-152573644 | 0.008 | LCE3C wholegene |
| Any | chr4: 15005553-15006221 | 0.01 | CPEB2 exon1 |
| Any | chr6: 27834340-27834967 | 0.01 | HIST1H1B exon1 |
| Critical | chr15: 22413530-22413968 | 0 | OR4N3P wholegene |
| Critical | chr15: 22382207-22382691 | 0 | OR4N4 wholegene |
| | | | LOC727924 wholegene |
| Critical | chr15: 22368319-22368667 | 0.001 | OR4M2 wholegene |
| Critical | chr2: 87420635-87420975 | 0.002 | ANAPC1 exon8 |
| Critical | chr22: 25436575-25437791 | 0.005 | KIAA1671 exon3 |
| Critical | chr1: 151337334-151337904 | 0.006 | SELENBP1 exon10 |
| | | | SELENBP1 exon8 |
| | | | SELENBP1 exon9 |
| Critical | chr1: 151371721-151372092 | 0.006 | PSMB4 wholegene |
| Critical | chr1: 151377013-151377600 | 0.006 | POGZ exon13 |
| | | | POGZ exon17 |
| | | | POGZ cxon19 |
| | | | POGZ exon18 |
| Critical | chr6: 32489367-32489781 | 0.007 | HLA-DRB5 exon2 |
| Endocrine | chr5: 177168045-177168772 | 0.001 | FAM153A exon6 |
| | | | FAM153A exon8 |
| Endocrine | chr1: 151337334-151337904 | 0.006 | SELENBP1 exon10 |
| | | | SELENBP1 exon8 |
| | | | SELENBP1 exon9 |
| Endocrine | chr1: 151371721-151372092 | 0.006 | PSMB4 wholegene |
| Endocrine | chr1: 151377013-151377600 | 0.006 | POGZ exon13 |
| | | | POGZ exon17 |
| | | | POGZ exon19 |
| | | | POGZ exon18 |
| Endocrine | chr1: 152586094-152586493 | 0.006 | LCE3B wholegene |
| Endocrine | chr7: 72430225-72430632 | 0.006 | NSUN5P2 exon4 |
| | | | TRIM74 exon4 |
| Endocrine | chr1: 152573024-152573644 | 0.009 | LCE3C wholegene |
| Flu-like | chr6: 31324250-31324461 | 0.004 | HLA-B exon1 |
| Flu-like | chr7: 5942016-5942639 | 0.007 | CCZ1 exon6 |
| Flu-like | chr2: 87420635-87420975 | 0.009 | ANAPC1 exon8 |
| Flu-like | chr2: 98161877-98162528 | 0.01 | ANKRD36B exon23 |
| Flu-like | chr3: 195390807-195391406 | 0.01 | SDHAP2 wholegene |
| Gastrointestinal | chr7: 100634945-100635138 | 0.001 | MUC12 exon2 |
| Gastrointestinal | chr19: 52149405-52149816 | 0.008 | SIGLEC14 exon2 |
| Gastrointestinal | chr1: 17087460-17087845 | 0.008 | MST1L exon2 |
| Gastrointestinal | chr5: 96248068-96248741 | 0.01 | ERAP2 exon16 |
| | | | ERAP2 exon15 |
| Musculoskeletal | chr21: 15013512-15014139 | 0.002 | POTED exon11 |
| Musculoskeletal | chr2: 87420635-87420975 | 0.005 | ANAPC1 exon8 |
| Musculoskeletal | chr7: 144015382-144015845 | 0.006 | OR2A1 exon1 |
| Musculoskeletal | chr17: 66265434-66265784 | 0.007 | SLC16A6 exon7 |
| | | | SLC16A6 exon6 |
| Musculoskeletal | chr17: 58078320-58078802 | 0.009 | TBC1D3P1-DHX40P1 exon6 |
| Multiple (grade ≥2) | chr5: 70307899-70309043 | 0.006 | NAIP wholegene |
| Multiple (grade ≥2) | chr14: 74003851-74004198 | 0.006 | ACOT1 wholegene |
| Multiple (grade ≥2) | chr14: 106207878-106208450 | 0.007 | IGHG1 exon4 |
| Multiple (grade ≥2) | chr19: 54804071-54804408 | 0.01 | LILRA3 wholegene |
| | | | LILRA6 wholegene |
| Neurologic | chr7: 100635139-100635510 | 0.001 | MUC12 exon2 |
| Neurologic | chr6: 29910896-29911491 | 0.001 | HLA-A wholegene |
| Neurologic | chr6: 29910586-29910895 | 0.001 | AK309533 exon1 |
| | | | HLA-A exon2 |
| | | | HLA-A exon1 |
| Neurologic | chr19: 44932636-44933058 | 0.002 | ZNF229 exon6 |
| Neurologic | chr7: 6825302-6825643 | 0.003 | RSPH10B2 exon16 |
| Neurologic | chr7: 6825302-6825643 | 0.003 | RSPH10B2 exon15 |
| Neurologic | chr7: 6825302-6825643 | 0.003 | RSPH10B2 exon9 |
| Neurologic | chr21: 15013512-15014139 | 0.003 | POTED exon11 |
| Neurologic | chr14: 45599029-45599398 | 0.004 | FKBP3 cxon3 |
| Thyroid | chr1: 151337334-151337904 | 0.002 | SELENBP1 exon10 |
| | | | SELENBP1 exon8 |
| | | | SELENBP1 exon9 |
| Thyroid | chr1: 151371721-151372092 | 0.002 | PSMB4 wholegene |
| Thyroid | chr1: 151377013-151377600 | 0.002 | POGZ exon13 |
| | | | POGZ exon17 |
| | | | POGZ exon19 |
| | | | POGZ cxon18 |
| Thyroid | chr5: 177168045-177168772 | 0.003 | FAM153A exon6 |
| | | | FAM153A exon8 |
| Thyroid | chr1: 152586494-152586889 | 0.006 | LCE3B cxon1 |
| Thyroid | chr16: 55853240-55853860 | 0.006 | CES1 exon7 |
| Thyroid | chr7: 72430225-72430632 | 0.007 | NSUN5P2 exon4 |
| | | | TRIM74 exon4 |
| Thyroid | chr2: 87423360-87423997 | 0.008 | ANAPC1 exon9 |
| Thyroid | chr10: 47416560-47417156 | 0.008 | FAM35DP exon5 |
| Multiple (any grade) | chr6: 31324462-31324741 | 0.004 | HLA-B wholegene |
| | | | HLA-B exon1 |
| Multiple (any grade) | chr6: 32634035-32634738 | 0.006 | HLA-DQB1 wholegene |
| Multiple (any grade) | chr7: 100634945-100635138 | 0.006 | MUC12 exon2 |
| Multiple (any grade) | chr6: 32628984-32629519 | 0.008 | HLA-DQB1 exon3 |
| | | | HLA-DQB1 exon4 |
| Multiple (any grade) | chr6: 32005948-32006709 | 0.008 | CYP21A2 wholegene |
| Multiple (any grade) | chr2: 87420635-87420975 | 0.01 | ANAPC1 exon8 |
| Multiple (any grade) | chr9: 135946828-135947422 | 0.01 | CEL exon11 |
| Skin | chr1: 40240339-40240976 | 0.003 | BMP8B exon2 |
| Skin | chr7: 144015382-144015845 | 0.006 | OR2A1 exon1 |
| Skin | chr7: 6012757-6013101 | 0.008 | PMS2 exon12 |
| | | | PMS2 exon14 |
| | | | PMS2 exon15 |
| | | | PMS2 exon11 |
| Skin | chr4: 69341748-69342476 | 0.009 | TMPRSS11E exon7 |
| Pulmonary | chr8: 7320000-7320652 | 0.006 | SPAG11B exon2 |

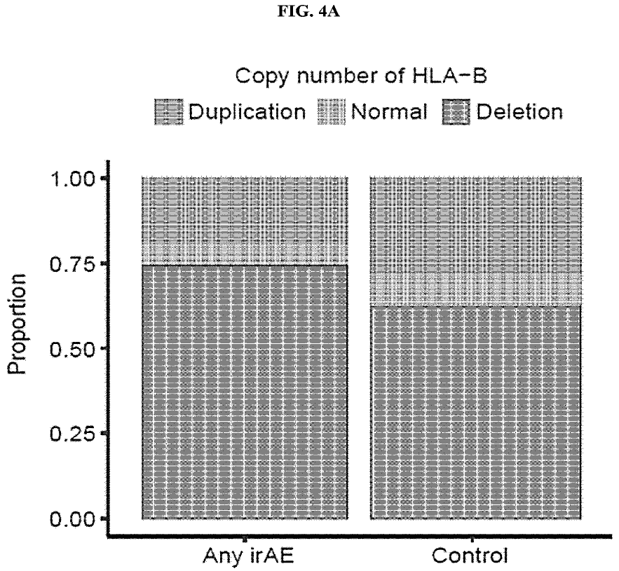

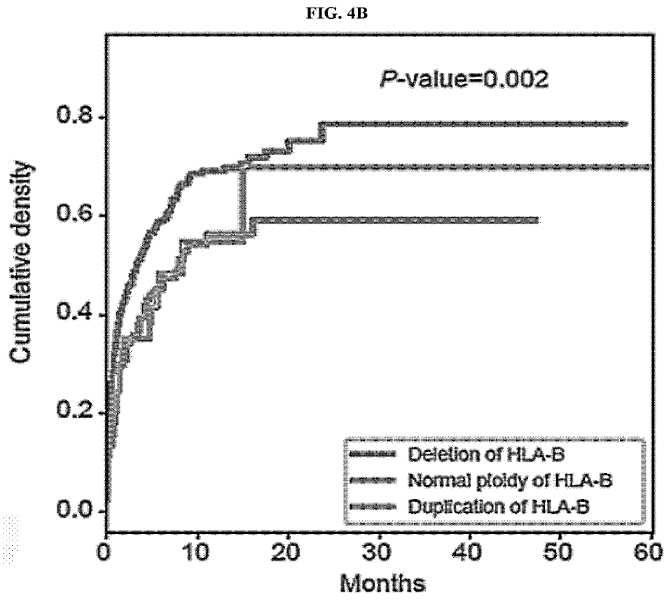

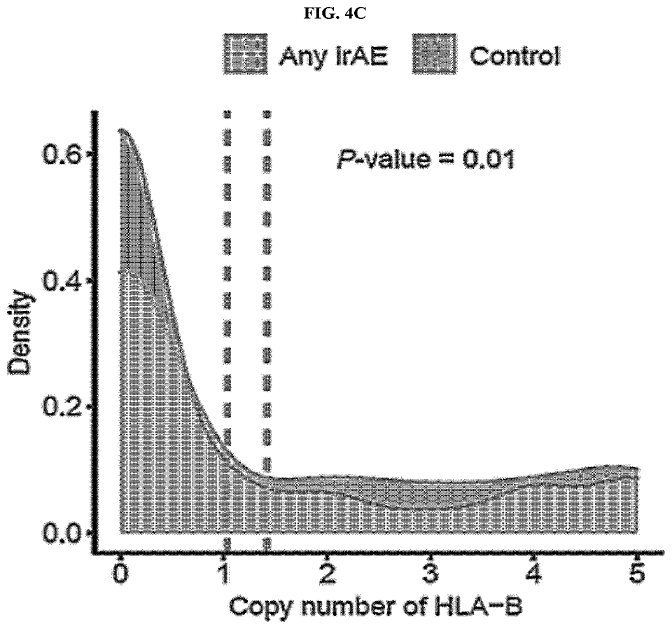

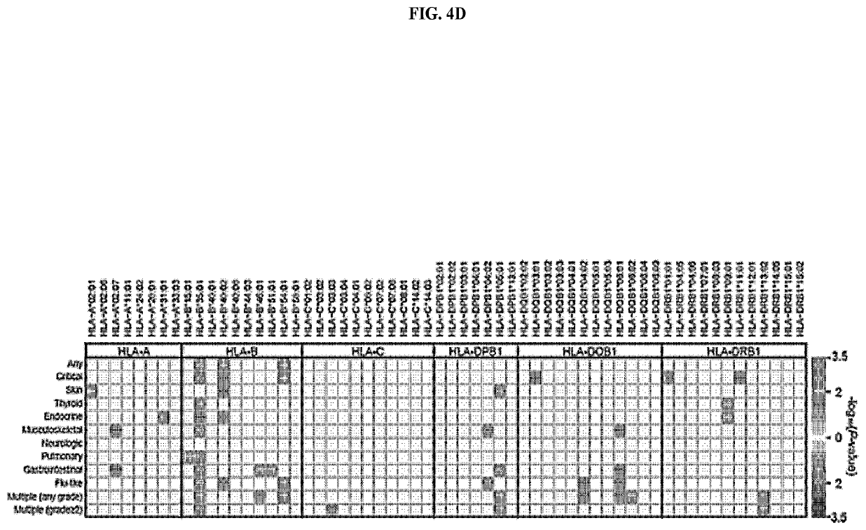

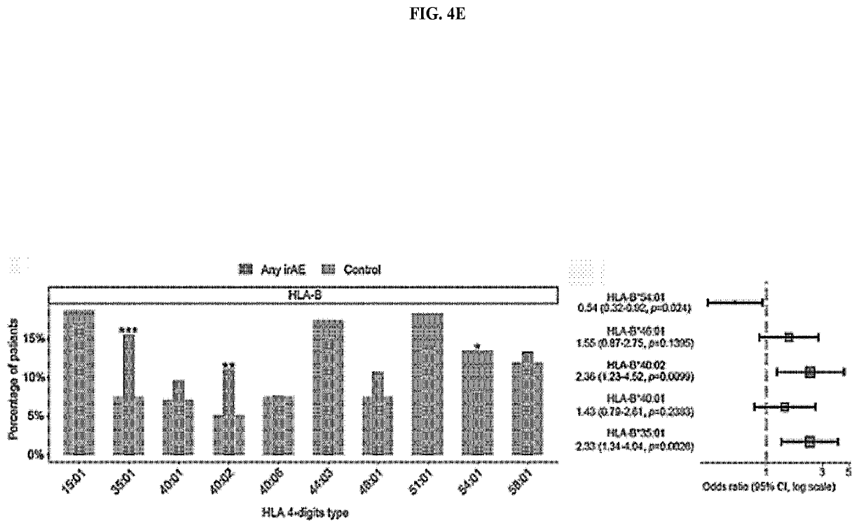

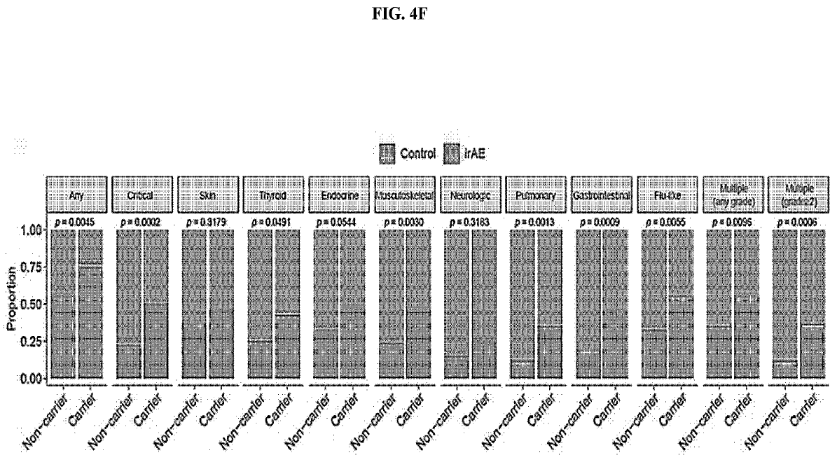

The most significant association with Any (odds ratio, 95% CI=0.72 (0.59-0.87), P=0.001) for CNV of HLA-B exon 2 (chr6: 31324462-31324741) encoding α1-domain that determines antigen-binding specificity was shown. To evaluate association with the irAE risk, all samples were divided into three groups according to a CNV status (i.e., deletion, normal ploidy, and duplication), and as shown in FIG. 4A, the three groups were differentially distributed between the irAE groups and the control. Particularly, as shown in FIG. 4B, HLA-B exon 2 deletion patients had a significantly shorter time to irAE occurrence (P=0.002, Cox proportional hazards model). In addition, as shown in FIG. 4C, the average copy number was significantly lower in irAE patients than non-irAE patients (P=0.01).

Meanwhile, previous studies have shown that various autoimmune diseases are associated with specific HLA alleles (Ahn, S et al., Immune Netw. 2011 December; 11 (6): 324-335.). Accordingly, Class I and II HLA typing was performed to evaluate the association between HLA alleles and 12 irAE labels using multivariate logistic regression, and HLA alleles which have an allele frequency higher than 3% in the cohort were used for subsequent analysis.

As a result of association evaluation, it was found that a specific allele of HLA-B and a specific HLA class II gene were associated with the predisposition of various irAEs. Particularly, as shown in FIGS. 4D and 4E, HLA-B*35:01 showed significant association (multivariate logistic regression P<0.05) for most irAE labels except Nervous system (P=0.27) and Skin (P=0.25).

As shown in FIG. 4F, HLA-B*35:01 increased the incidence of all irAE types, whereas the most significant increases were shown in Critical (chi square test, P=0.0002, non-carrier: 21% vs. carrier: 50%) and Multiple G>=2 (chi square test, P=0.0006, non-carrier: 12% vs. carrier: 37%). In addition to HLA-B*35:01, HLA-B*40:02 increased the risk of several irAE types, and HLA-B*54:01 decreased irAE incidence (refer to FIGS. 4D and 4E).

While HLA-B alleles or CNVs was associated with overall irAE risks, other variations showed specific correlations with a specific irAE label. For example, HLA-A duplication is particularly related to the nervous system. In Class II HLA genes, it was seen that HLA-DQB1 deletion is related to Multiple G>=1, whereas HLA-DRB5 duplication increases the risk of Critical. Other than the HLA gene, CNV of ANAPC1 had associations with various labels (Any, Critical, Flu-like, Musculoskeletal, Thyroid, and Multiple G>=1) (refer to Table 7). ANAPC1 protein belongs to various biological pathways, including cell cycle, mitosis, MHC class I-mediated antigen processing and presentation. In addition, this protein was identified as the one of 10 predictive biomarkers for immune evasion and immunotherapy response (Bou-Dargham, M. J et al., BMC Cancer 20, 572.). LCE3B and LCE3C deletion discovered to increase the risk of Any, Endocrine, and Thyroid-type irAEs had been previously reported to be related to psoriasis (Coto, E et al., BMC Med. Genet. 11, 45.), and the CNV of CYP21A2 showed significant association with the occurrence of Multiple G>=1. The copy number and genotype variations of a gene were reported in relation to the susceptibility of autoimmune diseases (Chen, I. X et al., Proc Natl Acad Sci USA. 2020 Sep. 22; 117 (38): 23684-23694.).

### Experimental Example 5. Integration Modeling of Germline SNVs for Prediction by irAE Type

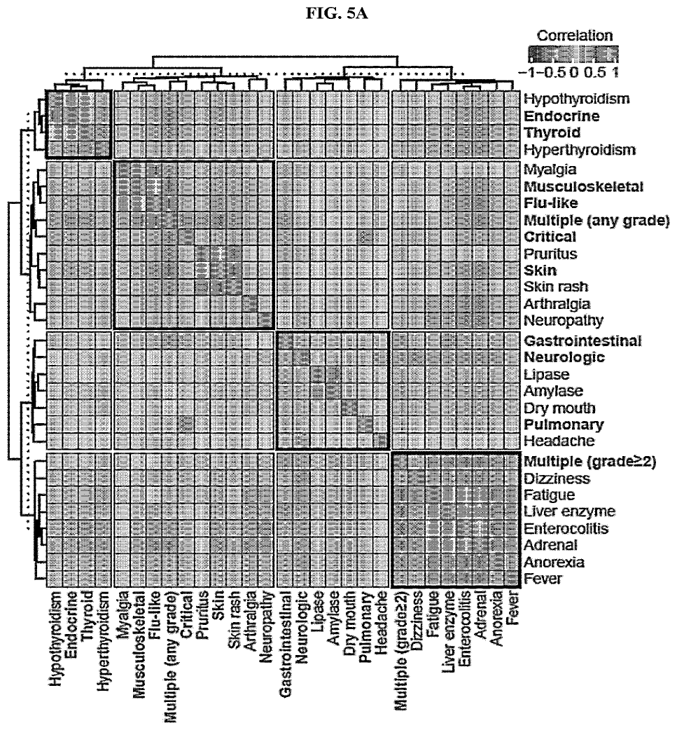

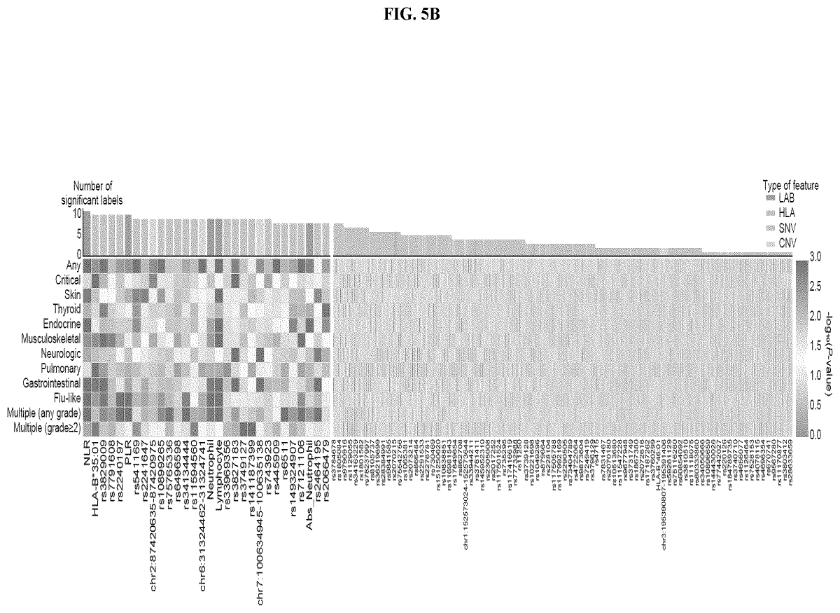

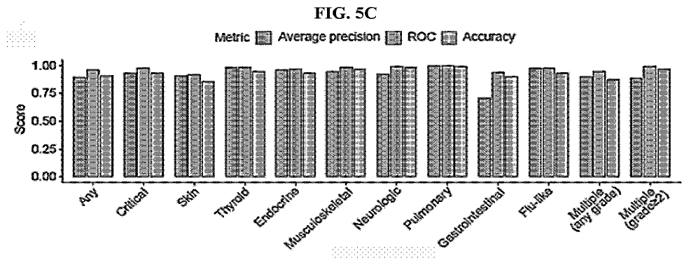

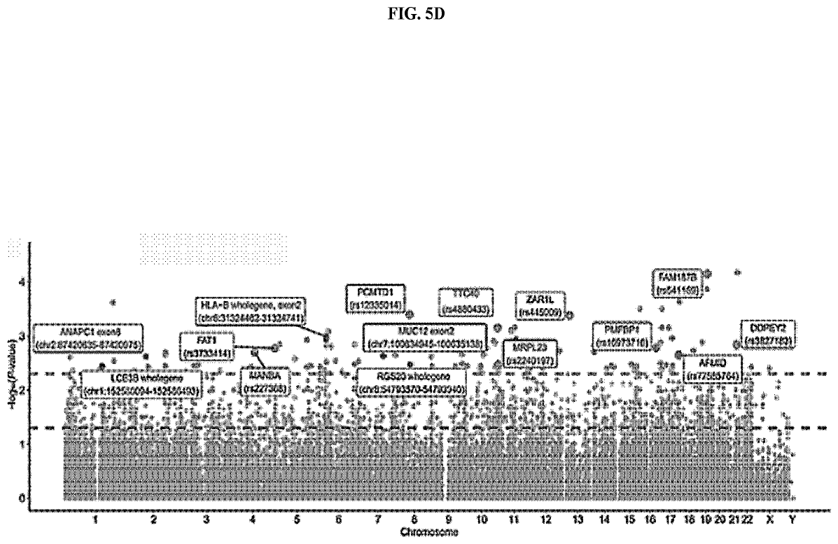

A more detailed role of SNV in forming pathological diversity of irAEs together with common risk factors such as immune cell fractions and HLA-B variation was postulated. To find genetic predisposition to various irAEs, multivariate logistic regression was performed using 119,688 non-synonymous SNVs with age, sex, and drug type as predictors for major irAE labels and related symptoms. And then, based on the association with 12,934 common SNVs, K-means clustering with a k value of 4 was performed on 29 irAE variables to observe the relationship of different irAEs sharing similar genetic components, which was shown in FIG. 5A. Items associated with the major irAE labels are highlighted in bold, and the color scale of cells is proportional to Pearson's correlation coefficient.

Afterward, a deep learning framework that trains a unified prediction model for each of the 12 major irAE labels using SNVs as input function along with laboratory data (refer to FIG. 2A), CNV (refer to Table 7), and HLA type (refer to FIG. 4D). A total of 859 features were used to train 12 irAE prediction models. Based on these 859 features, the relationship between various irAE types were investigated (refer to Table 8).

TABLE 8

| | | | | | | | | | | | | | | Number of |
|---|---|---|---|---|---|---|---|---|---|---|---|---|---|---|
| | Type of | | | | | Endo- | Musculo- | Neuro- | | Gastro- | | Multiple | Multiple | significant |
| Feature name | feature | Any | Critical | Skin | Thyroid | crine | skeletal | logic | Pulmonary | intestinal | Flu-like | (any grade) | (grade ≥2) | labels |
| NLR | LAB | 0.0001 | 0.0171 | 0.0013 | 0.0061 | 0.0005 | 0.0026 | 0.0051 | 0.1209 | 0.0000 | 0.0000 | 0.0000 | 0.0050 | 11 |
| HLA-B*35:01 | HLA | 0.0026 | 0.0005 | 0.2538 | 0.0177 | 0.0262 | 0.0023 | 0.2798 | 0.0011 | 0.0012 | 0.0032 | 0.0087 | 0.0014 | 10 |
| rs3829009 | SNV | 0.0011 | 0.0073 | 0.0446 | 0.0016 | 0.0078 | 0.0008 | 0.2082 | 0.7014 | 0.0007 | 0.0012 | 0.0021 | 0.0102 | 10 |
| rs7791608 | SNV | 0 0085 | 0 0372 | 0 0514 | 0.0139 | 0.0098 | 0 0016 | 0 0123 | 0 1834 | 0 0088 | 0.0080 | 0 0035 | 0.0066 | 10 |
| rs2240197 | SNV | 0.0034 | 0.2159 | 0.0121 | 0.0238 | 0.0133 | 0.0065 | 0.0369 | 0.0093 | 0.0239 | 0.0009 | 0.0004 | 0.1155 | 10 |
| PLR | LAB | 0.0027 | 0.0370 | 0.0060 | 0.0353 | 0.0130 | 0.0297 | 0.2456 | 0.4047 | 0.0045 | 0.0005 | 0.0004 | 0.0339 | 10 |
| rs541169 | SNV | 0.0001 | 0.1189 | 0.0018 | 0.0033 | 0.0023 | 0.0087 | 0.0471 | 0.2854 | 0.0169 | 0.0067 | 0.0195 | 0.9501 | 9 |
| rs2241647 | SNV | 0.0077 | 0.1778 | 0.0017 | 0.0289 | 0.0241 | 0.0428 | 0.0300 | 0.1521 | 0.0054 | 0.0043 | 0.0062 | 0.1248 | 9 |
| chr2:87420635-87420975 | CNV | 0.0024 | 0.0021 | 0.0322 | 0.0134 | 0.0216 | 0.0054 | 0.1353 | 0.0121 | 0.1745 | 0.0085 | 0.0096 | 0.0648 | 9 |
| rs10899265 | SNV | 0.0011 | 0.0255 | 0.0034 | 0.1525 | 0.0486 | 0.0238 | 0.1810 | 0.3163 | 0.0089 | 0.0032 | 0.0021 | 0.0351 | 9 |
| rs75765336 | SNV | 0.0103 | 0.0228 | 0.0884 | 0.0122 | 0.0103 | 0.0386 | 0.0105 | 0.1430 | 0.0083 | 0.0078 | 0.0008 | 0.3296 | 9 |
| rs6496598 | SNV | 0.0111 | 0.0121 | 0.0091 | 0.3479 | 0.2167 | 0.0046 | 0.0287 | 0.6502 | 0.0122 | 0.0162 | 0.0103 | 0.0288 | 9 |
| rs34134444 | SNV | 0.0047 | 0.2159 | 0.0121 | 0.0238 | 0.0133 | 0.0122 | 0.0751 | 0.0093 | 0.0239 | 0.0016 | 0.0008 | 0.1155 | 9 |
| rs11594560 | SNV | 0.0081 | 0.0162 | 0.0311 | 0.0083 | 0.0089 | 0.0438 | 0.0098 | 0.0754 | 0.0732 | 0.0533 | 0.0053 | 0.0017 | 9 |
| Neutrophil | LAB | 0.0211 | 0.2332 | 0.0274 | 0.0144 | 0.0042 | 0.0032 | 0.0176 | 0.4347 | 0.0013 | 0.0004 | 0.0012 | 0.0836 | 9 |
| Lymphocyte | LAB | 0.0000 | 0.0598 | 0.0000 | 0.0069 | 0.0002 | 0.0001 | 0.0582 | 0.4504 | 0.0000 | 0.0000 | 0.0000 | 0.0425 | 9 |
| rs33969356 | SNV | 0.0161 | 0.0146 | 0.0794 | 0.0257 | 0.0205 | 0.0207 | 0.1310 | 0.9996 | 0.0093 | 0.0196 | 0.0139 | 0.0076 | 9 |
| rs3827183 | SNV | 0.0015 | 0.0009 | 0.0283 | 0.0624 | 0.0264 | 0.0219 | 0.0005 | 0.1380 | 0.0006 | 0.0769 | 0.0036 | 0.0058 | 9 |
| rs3749127 | SNV | 0.0093 | 0.0185 | 0.0255 | 0.2733 | 0.1486 | 0.0096 | 0.1240 | 0.0482 | 0.0176 | 0.0286 | 0.0173 | 0.0002 | 9 |
| rs141187399 | SNV | 0.0807 | 0.0106 | 0.3054 | 0.0465 | 0.0130 | 0.0390 | 0.0417 | 0.5143 | 0.0495 | 0.0249 | 0.0116 | 0.0008 | 9 |
| chr7:100634945-100635138 | CNV | 0.0023 | 0.0489 | 0.0148 | 0.0614 | 0.0230 | 0.1603 | 0.0008 | 0.0774 | 0.0009 | 0.0206 | 0.0061 | 0.0342 | 9 |
| rs743923 | SNV | 0.0082 | 0.0046 | 0.0817 | 0.0243 | 0.0197 | 0.2344 | 0.0458 | 0.0363 | 0.0070 | 0.0135 | 0.0155 | 0.1715 | 9 |
| rs445909 | SNV | 0.0004 | 0.0429 | 0.0186 | 0.1009 | 0.0372 | 0.3982 | 0.0148 | 0.1695 | 0.0087 | 0.0356 | 0.0330 | 0.3469 | 8 |
| rs6511 | SNV | 0.0063 | 0.1817 | 0.0251 | 0.0368 | 0.0491 | 0.0189 | 0.0139 | 0.4471 | 0.3712 | 0.0185 | 0.0007 | 0.0820 | 8 |
| rs149324907 | SNV | 0.0045 | 0.0552 | 0.2600 | 0.0059 | 0.0019 | 0.1041 | 0.0108 | 0.0350 | 0.0239 | 0.0206 | 0.0031 | 0.4297 | 8 |
| rs7121106 | SNV | 0.0007 | 0.1418 | 0.0086 | 0.0073 | 0.0144 | 0.0009 | 0.0967 | 0.6368 | 0.0167 | 0.0024 | 0.0005 | 0.2196 | 8 |
| Abs_Neutrophil | LAB | 0.0017 | 0.0895 | 0.0456 | 0.0056 | 0.0002 | 0.0531 | 0.0103 | 0.3315 | 0.0065 | 0.0163 | 0.0034 | 0.0699 | 8 |
| rs2464195 | SNV | 0.0345 | 0.0356 | 0.0020 | 0.0587 | 0.0332 | 0.1712 | 0.0014 | 0.1326 | 0.0002 | 0.0071 | 0.0001 | 0.1031 | 8 |
| rs2066479 | SNV | 0.0037 | 0.0158 | 0.0109 | 0.0011 | 0.0024 | 0.0407 | 0.4046 | 0.2752 | 0.0530 | 0.0823 | 0.0199 | 0.0297 | 8 |
| rs3784678 | SNV | 0.0260 | 0.1042 | 0.0167 | 0.1162 | 0.0623 | 0.0028 | 0.0141 | 0.1527 | 0.0013 | 0.0033 | 0.0335 | 0.0291 | 8 |
| rs565791 | SNV | 0.0001 | 0.0778 | 0.0026 | 0.0073 | 0.0094 | 0.0082 | 0.1084 | 0.1317 | 0.0140 | 0.0170 | 0.0090 | 0.7509 | 8 |
| rs28477160 | SNV | 0.0287 | 0.0032 | 0.5431 | 0.0509 | 0.0339 | 0.0844 | 0.0170 | 0.0058 | 0.0120 | 0.0655 | 0.0157 | 0.0026 | 8 |
| rs76832527 | SNV | 0.0096 | 0.0228 | 0.0387 | 0.1703 | 0.0833 | 0.0339 | 0.0900 | 0.0180 | 0.0110 | 0.0582 | 0.0041 | 0.0430 | 8 |
| rs7641243 | SNV | 0.0012 | 0.0626 | 0.0030 | 0.0339 | 0.0247 | 0.0220 | 0.1098 | 0.1714 | 0.0197 | 0.0112 | 0.0264 | 0.0836 | 8 |
| rs3736930 | SNV | 0.0002 | 0.0144 | 0.0236 | 0.0123 | 0.0030 | 0.3045 | 0.1892 | 0.2275 | 0.0417 | 0.0295 | 0.0288 | 0.0852 | 8 |
| rs117192179 | SNV | 0.0200 | 0.0020 | 0.4828 | 0.0565 | 0.0283 | 0.0157 | 0.8065 | 0.0464 | 0.0217 | 0.0653 | 0.0430 | 0.0046 | 8 |
| rs8103849 | SNV | 0.0257 | 0.0107 | 0.4493 | 0.0888 | 0.0129 | 0.0455 | 0.0200 | 0.5103 | 0.0145 | 0.0248 | 0.0024 | 0.0773 | 8 |
| rs10221530 | SNV | 0.0605 | 0.0304 | 0.1287 | 0.0056 | 0.0132 | 0.0179 | 0.0050 | 1.0000 | 0.0497 | 0.0455 | 0.0232 | 0.1528 | 8 |
| rs11854484 | SNV | 0.0312 | 0.0038 | 0.1490 | 0.0512 | 0.0639 | 0.0136 | 0.0475 | 0.1316 | 0.0362 | 0.0125 | 0.0437 | 0.0206 | 8 |
| rs1805084 | SNV | 0.0772 | 0.0070 | 0.2491 | 0.0015 | 0.0114 | 0.0493 | 0.3598 | 0.0080 | 0.0471 | 0.1406 | 0.0098 | 0.0196 | 8 |
| rs76638917 | SNV | 0.0074 | 0.0286 | 0.0079 | 0.0923 | 0.0142 | 0.0157 | 0.0723 | 0.4617 | 0.0081 | 0.0250 | 0.0057 | 0.1813 | 8 |
| rs117134739 | SNV | 0.0167 | 0.0116 | 0.0016 | 0.1095 | 0.0945 | 0.0039 | 0.0091 | 0.0247 | 0.1141 | 0.0131 | 0.0253 | 0.0886 | 8 |
| rs3803824 | SNV | 0.0071 | 0.0201 | 0.0245 | 0.3750 | 0.7506 | 0.0034 | 0.3688 | 0.0270 | 0.0037 | 0.0669 | 0.0203 | 0.0496 | 8 |
| rs9908414 | SNV | 0.0003 | 0.0185 | 0.0086 | 0.0025 | 0.0027 | 0.0041 | 0 8586 | 0.0330 | 0.0573 | 0.0056 | 0.0774 | 0.0710 | 8 |
| rs2305440 | SNV | 0.0037 | 0.0154 | 0.0070 | 0.2122 | 0.1141 | 0.0040 | 0.0518 | 0.5404 | 0.0270 | 0.0070 | 0.0123 | 0.0495 | 8 |
| rs116602139 | SNV | 0.02.23 | 0.0104 | 0.1855 | 0.0270 | 0.0752 | 0.0962 | 0.0248 | 0.0550 | 0.0101 | 0.0138 | 0.0466 | 0.0178 | 8 |
| rs12516846 | SNV | 0.0262 | 0.0030 | 0.0709 | 0.7326 | 0.1928 | 0.0478 | 0.8069 | 0.0415 | 0.0002 | 0.0066 | 0.0090 | 0.0011 | 8 |
| rs60927391 | SNV | 0.0300 | 0.2697 | 0.0627 | 0.0058 | 0.0066 | 0.0380 | 0.0609 | 0.8223 | 0.0437 | 0.0098 | 0.0304 | 0.0814 | 7 |
| rs9790916 | SNV | 0.1957 | 0.0488 | 0.6394 | 0.7282 | 0.9913 | 0.0039 | 0.0027 | 0.3365 | 0.0038 | 0.0162 | 0.0136 | 0.0027 | 7 |
| rs3774729 | SNV | 0.0321 | 0.0126 | 0.0175 | 0.0070 | 0.0187 | 0.3955 | 0.0522 | 0.4423 | 0.0243 | 0.1213 | 0.0085 | 0.0893 | 7 |
| rs61729307 | SNV | 0.0013 | 0.0270 | 0.0533 | 0.0167 | 0.0071 | 0.0612 | 0.1634 | 0.2269 | 0.0245 | 0.0164 | 0.0031 | 1.0000 | 7 |
| rs141716191 | SNV | 0.0397 | 0.0400 | 0.0528 | 0.6440 | 0.7035 | 0.1494 | 0 1685 | 0.0069 | 0.0119 | 0.0197 | 0.0416 | 0 0090 | 7 |
| rs1944294 | SNV | 0.0342 | 0.4292 | 0.0442 | 0.0245 | 0.0144 | 0.0109 | 0.6950 | 0.7950 | 0.1532 | 0.0043 | 0.0014 | 0.3344 | 7 |
| rs7553399 | SNV | 0.1476 | 0.0023 | 0.3543 | 0.0509 | 0.0248 | 0.0361 | 0.0110 | 0.0479 | 0.2585 | 0.1246 | 0.0036 | 0.0016 | 7 |
| rs3813803 | SNV | 0.0044 | 0.0010 | 0.1132 | 0.0096 | 0.0032 | 0.1033 | 0.0105 | 0.2929 | 0.0445 | 0.2230 | 0.1007 | 0.0285 | 7 |
| rs11146955 | SNV | 0.0149 | 0.0195 | 0.6666 | 0.1900 | 0.0683 | 0.0010 | 0.1628 | 0.1047 | 0.0234 | 0.0003 | 0.0330 | 0.0101 | 7 |
| rs4752904 | SNV | 0.2855 | 0.0191 | 0.0353 | 0.4678 | 0.3706 | 0.0016 | 0.0111 | 0.1507 | 0.0958 | 0.0101 | 0.0077 | 0.0001 | 7 |
| rs146824779 | SNV | 0.0188 | 0.0030 | 0.0833 | 0.7859 | 0.4266 | 0.0261 | 0.0507 | 0.0003 | 0.0597 | 0.0107 | 0.0124 | 0.0040 | 7 |
| rs1129055 | SNV | 0.2180 | 0.0240 | 0.0280 | 0.1015 | 0.1184 | 0.4662 | 0.0209 | 0.0015 | 0.0058 | 0.0536 | 0.0325 | 0.0350 | 7 |
| rs1047099 | SNV | 0.0148 | 0.0118 | 0.0151 | 0.0867 | 0.0084 | 0.0475 | 0.6139 | 0.5040 | 0.3770 | 0.1684 | 0.0438 | 0.0185 | 7 |
| rs10508 | SNV | 0.0003 | 0.1399 | 0.0009 | 0.0110 | 0.0018 | 0.0088 | 0.6374 | 0.4405 | 0.0852 | 0.0143 | 0.0112 | 0.3370 | 7 |
| rs77585764 | SNV | 0.0023 | 0.0465 | 0.0018 | 0.0972 | 0.0482 | 0.3693 | 0.6057 | 0.3505 | 0.0070 | 0.0557 | 0.0047 | 0.0015 | 7 |
| rs7693453 | SNV | 0.0097 | 0.0092 | 0.1802 | 0.0400 | 0.0191 | 0.0025 | 0.1668 | 0.1137 | 0.0635 | 0.0037 | 0.0405 | 0.0870 | 7 |
| rs117936881 | SNV | 0.0466 | 0.0102 | 0.0921 | 0.0082 | 0.0069 | 0.2702 | 0.3648 | 0.0453 | 0.2298 | 0.4917 | 0.0300 | 0.0222 | 7 |
| rs74611066 | SNV | 0.0392 | 0.0081 | 0.0970 | 0 0321 | 0.0019 | 0.9977 | 0.3862 | 0.0023 | 0.2055 | 0.3497 | 0 0260 | 0.0149 | 7 |
| rs12139100 | SNV | 0.0104 | 0.0547 | 0.0076 | 0.0054 | 0.0060 | 0.0507 | 0.6308 | 0.3085 | 0.4335 | 0.0490 | 0.0229 | 0.0003 | 7 |
| rs184750798 | SNV | 0.0087 | 0.0018 | 0.8991 | 0.1096 | 0.0612 | 0.0042 | 0.1548 | 0.7011 | 0.0002 | 0.0062 | 0.0016 | 0.0005 | 7 |
| rs34670941 | SNV | 0.1558 | 0.0300 | 0.0129 | 0.0412 | 0.1138 | 0.0717 | 0.0384 | 0.2901 | 0.0274 | 0.4398 | 0.0072 | 0.0098 | 7 |
| rs34163229 | SNV | 0.0022 | 0.0313 | 0.0245 | 0.5586 | 0.6077 | 0.0495 | 0.0370 | 0.0928 | 0.0123 | 0.0010 | 0.0582 | 0.0701 | 7 |
| rs1056523 | SNV | 0.1668 | 0.1151 | 0.6663 | 0.0276 | 0.0318 | 0.0192 | 0.3541 | 0.0886 | 0.0077 | 0.0079 | 0.0104 | 0.0347 | 7 |
| rs117318814 | SNV | 0.0501 | 0.1166 | 0.5041 | 0.0217 | 0.0079 | 0.1235 | 0.9742 | 0.0121 | 0.0443 | 0.0363 | 0.0045 | 0.0051 | 7 |
| rs9652296 | SNV | 0.0040 | 0.0098 | 0.0928 | 0.0341 | 0.0064 | 0.0611 | 0.3965 | 0.0052 | 0.0411 | 0.0682 | 0.0092 | 0.0565 | 7 |
| rs9503233 | SNV | 0.0118 | 0.0152 | 0.0205 | 0 1843 | 0.0509 | 0.0214 | 0.1080 | 0.0150 | 0.0048 | 0.0710 | 0 0657 | 0.0199 | 7 |
| rs117139389 | SNV | 0.1173 | 0.0039 | 0.0840 | 0.0037 | 0.0278 | 0.0591 | 0.0203 | 0.2015 | 0.0062 | 0.2261 | 0.0273 | 0.0013 | 7 |
| rs11479 | SNV | 0.0099 | 0.0030 | 0.0940 | 0.4256 | 0.1986 | 0.0107 | 0.0694 | 0.4263 | 0.0043 | 0.0027 | 0.0069 | 0.0119 | 7 |
| rs2278132 | SNV | 0.0309 | 0.0136 | 0.2565 | 0.0033 | 0.0091 | 0.3885 | 0.4476 | 0.7449 | 0.0446 | 0.3187 | 0.0101 | 0.0166 | 7 |
| rs1851725 | SNV | 0.0018 | 0.0320 | 0.0517 | 0.0273 | 0.0187 | 0.0490 | 0.0965 | 0.3340 | 0.1582 | 0.0471 | 0.0191 | 0.3775 | 7 |
| rs2071762 | SNV | 0.0088 | 0.5509 | 0.0353 | 0.0199 | 0.0201 | 0.0018 | 0.8829 | 0.5884 | 0.8574 | 0.0241 | 0.0465 | 0.4789 | 7 |
| rs1801582 | SNV | 0.0438 | 0.0242 | 0.1521 | 0.1684 | 0.0509 | 0.1105 | 0.0287 | 0.3298 | 0.0233 | 0.0490 | 0.0062 | 0.0305 | 7 |
| rs72663752 | SNV | 0.0267 | 0.0985 | 0.0514 | 0.3527 | 0.4968 | 0.0139 | 0.0393 | 0.7824 | 0.0153 | 0.0031 | 0.0107 | 0.0066 | 7 |
| rs75680863 | SNV | 0.0150 | 0.0735 | 0.0405 | 0 0260 | 0.0063 | 0.0235 | 0.3312 | 0.1719 | 0.0586 | 0.0107 | 0 0341 | 0.1543 | 7 |
| rs3732215 | SNV | 0.0307 | 0.0385 | 0.0327 | 0.2255 | 0.1395 | 0.0430 | 0.0020 | 0.1388 | 0.0736 | 0.0236 | 0.0050 | 0.2634 | 7 |
| rs1015149 | SNV | 0.0008 | 0.0418 | 0.1479 | 0.0625 | 0.0283 | 0.0623 | 0.0254 | 0.0794 | 0.0388 | 0.0125 | 0.0013 | 0.1633 | 7 |
| rs58905020 | SNV | 0.0435 | 0.0797 | 0.0027 | 0.3154 | 0.1210 | 0.0104 | 0.1013 | 0.3661 | 0.0077 | 0.0028 | 0.0043 | 0.0023 | 7 |
| rs11552724 | SNY | 0.0036 | 0.0067 | 0.0633 | 0.0101 | 0.0036 | 0.1447 | 0.6079 | 0.0453 | 0.0397 | 0.1561 | 0.0216 | 0.2105 | 7 |
| rs554324677 | SNV | 0.0295 | 0.0273 | 0.0498 | 0.0191 | 0.0100 | 0.2133 | 0.0174 | 0.0823 | 0.4563 | 0.2765 | 0.0238 | 0.1885 | 7 |
| rs923828 | SNV | 0.0204 | 0.2651 | 0.0034 | 0.6156 | 0.9373 | 0.0007 | 0.0405 | 0.1622 | 0.0288 | 0.0397 | 0.2584 | 0.0062 | 7 |
| rs142854953 | SNV | 0.0853 | 0.2126 | 0.0322 | 0.0144 | 0.0406 | 0.0239 | 0.0181 | 0.7475 | 0.5160 | 0.1402 | 0.0231 | 0.0020 | 7 |
| rs78237997 | SNV | 0.2120 | 0.0094 | 0.8601 | 0 0391 | 0.0500 | 0.0418 | 0.0418 | 0.1420 | 0.0215 | 0.1285 | 0 0262 | 0.0036 | 7 |
| rs2270581 | SNV | 0.0075 | 0.0762 | 0.0007 | 0.3618 | 0.2648 | 0.0051 | 0.0192 | 0.3693 | 0.0215 | 0.0157 | 0.0110 | 0.1643 | 7 |
| rs79298408 | SNV | 0.0638 | 0.0053 | 0.4488 | 0.1006 | 0.0765 | 0.0098 | 0.0129 | 0.2282 | 0.0032 | 0.0194 | 0.0039 | 0.0008 | 7 |
| rs2278196 | SNV | 0.0102 | 0.1758 | 0.0277 | 0.1134 | 0.0313 | 0.0112 | 0.0189 | 0.5458 | 0.3329 | 0.0689 | 0.0071 | 0.0167 | 7 |
| chr7:100635139-100635510 | CNV | 0.0066 | 0.2022 | 0.0366 | 0.0722 | 0.0388 | 0.1103 | 0.0007 | 0.1483 | 0.0016 | 0.0194 | 0.0063 | 0.0685 | 7 |
| rs67383011 | SNV | 0.0087 | 0.0545 | 0.0021 | 0.3868 | 0.3468 | 0.1495 | 0.2249 | 0.0122 | 0.0172 | 0.0375 | 0.0131 | 0.1733 | 6 |
| rs2303926 | SNV | 0.0023 | 0.5952 | 0.0004 | 0.0003 | 0.0032 | 0.2853 | 0.6220 | 0.9051 | 0.0433 | 0.2285 | 0.0113 | 0.6935 | 6 |
| rs2289591 | SNV | 0.0516 | 0.0042 | 0.0301 | 0.6492 | 0.6837 | 0.0596 | 0.100 | 0.6190 | 0.0035 | 0.0463 | 0.0447 | 0.0098 | 6 |
| rs78572085 | SNV | 0.0493 | 0.0077 | 0.5790 | 0.0595 | 0.1025 | 0.0095 | 0.5778 | 0.4706 | 0.0311 | 0.0369 | 0.0528 | 0.0374 | 6 |
| rs3733617 | SNV | 0.0276 | 0.3404 | 0.0964 | 0.0936 | 0.0772 | 0.0311 | 0.0685 | 0.6021 | 0.0024 | 0.0458 | 0.0007 | 0.0040 | 6 |
| rs8105737 | SNV | 0.0132 | 0.3054 | 0.2896 | 0.0096 | 0.0084 | 0.0212 | 0.9331 | 0.3780 | 0.0324 | 0.0823 | 0.0328 | 0.7025 | 6 |
| rs1053338 | SNV | 0.0049 | 0.0384 | 0.0122 | 0.0820 | 0.0443 | 0.0676 | 0.2225 | 0.9422 | 0.1265 | 0.0070 | 0.0016 | 0.3141 | 6 |
| rs2304974 | SNV | 0.0527 | 0.0148 | 0.4677 | 0.0026 | 0.0029 | 0.4282 | 0.0878 | 0.4061 | 0.0135 | 0.5108 | 0.0283 | 0.0156 | 6 |
| rs9899177 | SNV | 0.0007 | 0.1573 | 0.0035 | 0.0138 | 0.0040 | 0.0036 | 0.6994 | 0.1301 | 0.2221 | 0.0022 | 0.0735 | 0.2559 | 6 |
| rs34069439 | SNV | 0.0069 | 0.1848 | 0.0078 | 0.0387 | 0.0125 | 0.2156 | 0.0093 | 0.5074 | 0.0657 | 0.3096 | 0.0148 | 0.6677 | 6 |
| rs72483216 | SNV | 0.0069 | 0.0435 | 0.1171 | 0.0370 | 0.0090 | 0.0962 | 0.3353 | 0.1676 | 0.2088 | 0.0320 | 0.0097 | 0.2461 | 6 |
| rs3822214 | SNV | 0.0957 | 0.2087 | 0.1635 | 0.5529 | 0.6380 | 0.0091 | 0.0085 | 0.0395 | 0.1268 | 0.0328 | 0.0449 | 0.0046 | 6 |
| rs35019975 | SNV | 0.0115 | 0.0448 | 0.1627 | 0.0163 | 0.0096 | 0.4562 | 0.0828 | 0.4032 | 0.0682 | 0.0878 | 0.0076 | 0.0026 | 6 |
| rs75455499 | SNV | 0.1170 | 0.0228 | 0.1515 | 0.8276 | 0.9438 | 0.0494 | 0.0390 | 0.0967 | 0.0493 | 0.0287 | 0.0059 | 0.0871 | 6 |
| rs17089782 | SNV | 0.0288 | 0.1394 | 0.0396 | 0.0015 | 0.0112 | 0.1965 | 0.1315 | 0.8920 | 0.1485 | 0.2463 | 0.0397 | 0.0374 | 6 |
| rs36219699 | SNV | 0.0426 | 0.0737 | 0.2314 | 0.1619 | 0.1162 | 0.0452 | 0.8783 | 0.7927 | 0.0174 | 0.0181 | 0.0083 | 0.0378 | 6 |
| rs1713420 | SNV | 0.0092 | 0.1569 | 0.0142 | 0.1430 | 0.2193 | 0.0785 | 0.0634 | 0.4755 | 0.0210 | 0.0243 | 0.0369 | 0.0006 | 6 |
| rs78721070 | SNV | 0.0291 | 0.0038 | 0.0150 | 0.1280 | 0.0423 | 0.1340 | 0.6549 | 0.0058 | 0.6238 | 0.0543 | 0.0183 | 0.2176 | 6 |
| rs139253533 | SNV | 0.2448 | 0.0531 | 0.0917 | 0.9165 | 0.4577 | 0.0358 | 0.0006 | 0.8417 | 0.0398 | 0.0200 | 0.0300 | 0.0135 | 6 |
| rs17128275 | SNV | 0.0096 | 0.1368 | 0.0265 | 0.0036 | 0.0142 | 0.2301 | 0.5973 | 0.1614 | 0.0323 | 0.0263 | 0.0593 | 0.0976 | 6 |
| rs308998 | SNV | 0.0148 | 0.0139 | 0.1719 | 0.0029 | 0.0112 | 0.0349 | 0.3350 | 0.0093 | 0.3142 | 0.1063 | 0.2042 | 0.0903 | 6 |
| rs35751778 | SNV | 0.0870 | 0.0722 | 0.0512 | 0.2417 | 0.1699 | 0.0059 | 0.0338 | 0.3507 | 0.0276 | 0.0159 | 0.0252 | 0.0268 | 6 |
| rs11458 | SNV | 0.0428 | 0.0354 | 0.1577 | 0.1651 | 0.0932 | 0.0857 | 0.0071 | 0.1271 | 0.0374 | 0.0471 | 0.0032 | 0.1097 | 6 |
| rs76688635 | SNV | 0.0177 | 0.0014 | 0.7789 | 0.0619 | 0.0484 | 0.4012 | 0.6550 | 0.0035 | 0.0345 | 0.3453 | 0.0371 | 0.2135 | 6 |
| rs1141509 | SNV | 0.0061 | 0.3018 | 0.0047 | 0.3203 | 0.2133 | 0.0083 | 0.1872 | 0.8834 | 0.0296 | 0.0131 | 0.0033 | 0.7693 | 6 |
| rs28384991 | SNV | 0.0044 | 0.0555 | 0.0252 | 0.0132 | 0.0034 | 0.2292 | 0.3193 | 0.6316 | 0.0601 | 0.0754 | 0.0042 | 0.0264 | 6 |
| rs77125878 | SNV | 0.2005 | 0.1185 | 0.0690 | 0.1103 | 0.1115 | 0.0360 | 0.0487 | 0.2848 | 0.0021 | 0.0086 | 0.0066 | 0.0046 | 6 |
| rs190713831 | SNV | 0.1287 | 0.0749 | 0.1591 | 0.0277 | 0.0472 | 0.1537 | 0.1109 | 0.8409 | 0.0003 | 0.0060 | 0.0075 | 0.0015 | 6 |
| rs2189473 | SNV | 0.0064 | 0.0556 | 0.0383 | 0.0427 | 0.0252 | 0.2606 | 0.1616 | 0.6843 | 0.0506 | 0.0460 | 0.0206 | 0.1637 | 6 |
| rs12647859 | SNV | 0.0096 | 0.0977 | 0.0735 | 0.0668 | 0.0201 | 0.0035 | 0.0365 | 0.0396 | 0.3085 | 0.0043 | 0.0504 | 0.0629 | 6 |
| rs117016551 | SNV | 0.0798 | 0.0225 | 0.1326 | 0.1236 | 0.0699 | 0.0113 | 0.2318 | 0.7900 | 0.0358 | 0.0273 | 0.0065 | 0.0223 | 6 |
| rs919364 | SNV | 0.0114 | 0.9638 | 0.0163 | 0.0499 | 0.0437 | 0.0048 | 0.8103 | 0.6739 | 0.7909 | 0.0329 | 0.6216 | 0.2536 | 6 |
| rs80359065 | SNV | 0 0315 | 0.0125 | 0.0799 | 0.0094 | 0.0165 | 0.0847 | 1.0000 | 0.0604 | 0.0699 | 0 1486 | 0.0388 | 0.0432 | 6 |
| rs2071834 | SNV | 0.0208 | 0.0231 | 0.0092 | 0.4396 | 0.3666 | 0.3310 | 0.0834 | 0.8013 | 0.0026 | 0.0776 | 0.0016 | 0.0346 | 6 |
| rs3746887 | SNV | 0.0001 | 0.1013 | 0.0170 | 0.0038 | 0.0130 | 0.1019 | 0.0764 | 0.0660 | 0.3492 | 0.0429 | 0.0154 | 0.5135 | 6 |
| rs9841585 | SNV | 0.0059 | 0.1270 | 0.0884 | 0.0214 | 0.0384 | 0.1251 | 0.0005 | 0.2141 | 0.0317 | 0.2217 | 0.0380 | 0.5456 | 6 |
| rs3182535 | SNV | 0.0406 | 0.2985 | 0.0487 | 0.0498 | 0.0136 | 0.1664 | 0.0918 | 0.4467 | 0.0025 | 0.1711 | 0.0169 | 0.3162 | 6 |
| rs1875272 | SNV | 0.0285 | 0.4570 | 0.0604 | 0.0208 | 0.0472 | 0.0801 | 0.4849 | 0.6186 | 0.0491 | 0.0095 | 0.0266 | 0.0745 | 6 |
| rs10857469 | SNV | 0.0094 | 0.8805 | 0.1100 | 0.6106 | 0.5146 | 0.0184 | 0.0004 | 0.3338 | 0.0028 | 0.0033 | 0.0264 | 0.3266 | 6 |
| rs2297654 | SNV | 0.0558 | 0.1115 | 0.3966 | 0.0438 | 0.0231 | 0.0207 | 0.4876 | 0.7737 | 0.0016 | 0.0241 | 0.0075 | 0.0917 | 6 |
| rs987495 | SNV | 0.0013 | 0.2517 | 0.0329 | 0.0610 | 0.0808 | 0.0064 | 0.2957 | 0.0137 | 0.0429 | 0.0051 | 0.3667 | 0.3792 | 6 |
| rs74777754 | SNV | 0.0042 | 0.0098 | 0.0193 | 0.0063 | 0.0071 | 0.1813 | 0.1490 | 0.0008 | 0.1063 | 0.3065 | 0.0980 | 0.0544 | 6 |
| rs7570797 | SNV | 0.0306 | 0.0253 | 0.1097 | 0.0795 | 0.0102 | 0.0082 | 0.2656 | 0.0303 | 0.8998 | 0.0123 | 0.2037 | 0.2036 | 6 |
| rs9657518 | SNV | 0.0104 | 0.0538 | 0.1101 | 0.0132 | 0.0032 | 0.0539 | 0.2336 | 0.7964 | 0.0021 | 0.2979 | 0.0254 | 0.0058 | 6 |
| rs3752978 | SNV | 0.0223 | 0.0433 | 0.0402 | 0.0006 | 0.0008 | 0.0668 | 0.2597 | 0.9719 | 0.1423 | 0.1394 | 0.0499 | 0.0886 | 6 |
| rs2070215 | SNV | 0.0194 | 0.0596 | 0.0226 | 0.1051 | 0.0210 | 0.5639 | 0.0134 | 0.8204 | 0.0018 | 0.2627 | 0.0040 | 0.1956 | 6 |
| rs4880433 | SNV | 0.0007 | 0.3256 | 0.0395 | 0.5797 | 0.0425 | 0.0036 | 0.4336 | 0.0127 | 0.1473 | 0.0258 | 0.1139 | 0.2373 | 6 |
| RBC | LAB | 0.0000 | 0.0769 | 0.0021 | 0.0547 | 0.0114 | 0.1074 | 0.3021 | 0.7251 | 0.0144 | 0.0097 | 0.0013 | 0.5371 | 6 |
| Protein | LAB | 0.0000 | 0.0175 | 0.0065 | 0.4421 | 0.0810 | 0.2047 | 0.4991 | 0.2477 | 0.0344 | 0.0165 | 0.0103 | 0.4327 | 6 |
| rs1043879 | SNV | 0.0604 | 0.0098 | 0.1210 | 0.0300 | 0.0369 | 0.0837 | 0.0116 | 0.0091 | 0.0599 | 0.1471 | 0.0342 | 0.1276 | 6 |
| HLA-B*40:02 | HLA | 0.0099 | 0.0358 | 0.0319 | 0.0506 | 0.0477 | 0.5031 | 0.3574 | 0.0293 | 0.9574 | 0.0456 | 0.1889 | 0.3164 | 6 |
| rs74846385 | SNV | 0.0062 | 0.0914 | 0.0340 | 0.0340 | 0.0693 | 0.0160 | 0.2587 | 0.6336 | 0.0525 | 0.0161 | 0.0058 | 0.4625 | 6 |
| rs142457134 | SNV | 0.0341 | 0.0234 | 0.6624 | 0.0003 | 0.0025 | 0.2284 | 0.0043 | 0.0551 | 0.0919 | 0.3740 | 0.0161 | 0.0622 | 6 |
| rs7128017 | SNV | 0.0107 | 0.0060 | 0.1383 | 0.2341 | 0.1175 | 0.0007 | 0.2945 | 0.9483 | 0.0775 | 0.0082 | 0.0035 | 0.0121 | 6 |
| rs1378796 | SNV | 0.0045 | 0.1799 | 0.0352 | 0.0033 | 0.0064 | 0.0924 | 0.1646 | 0.3160 | 0.0802 | 0.0404 | 0.3928 | 0.0143 | 6 |
| rs75542756 | SNV | 0.0154 | 0.2547 | 0.03$2 | 0.0507 | 0.0603 | 0.0093 | 0.1025 | 0.5961 | 0.0229 | 0.0021 | 0.0258 | 0.0976 | 6 |
| rs12335014 | SNV | 0.0004 | 0.0892 | 0.0096 | 0.0423 | 0.0011 | 0.1551 | 0.3229 | 0.0752 | 0.1814 | 0.0237 | 0.0207 | 0.6433 | 6 |
| rs260462 | SNV | 0.4334 | 0.3129 | 0.0798 | 0.9178 | 0.9811 | 0.0006 | 0.0438 | 0.9189 | 0.0064 | 0.0012 | 0.0311 | 0.0123 | 6 |
| rs4667591 | SNV | 0.0023 | 0.0880 | 0.0691 | 0.0245 | 0.0356 | 0.1779 | 0.5707 | 0.3371 | 0.2018 | 0.0411 | 0.0263 | 0.7614 | 5 |
| rs1800205 | SNV | 0.0362 | 0.0846 | 0.1279 | 0.1807 | 0.0928 | 0.0133 | 0.8093 | 0.3904 | 0.0647 | 0.0135 | 0.0078 | 0.0054 | 5 |
| rs17409304 | SNV | 0.1670 | 0.0390 | 0.2982 | 0.2612 | 0.2855 | 0.0170 | 0.2321 | 0.1355 | 0.0043 | 0.0455 | 0.0036 | 0.0817 | 5 |
| rs71425650 | SNV | 0.0213 | 0.2264 | 0.0598 | 0.0145 | 0.0102 | 0.0807 | 0.1146 | 0.6604 | 0.3212 | 0.0874 | 0.0140 | 0.0141 | 5 |
| rs2075252 | SNV | 0.0020 | 0.0874 | 0.0647 | 0.0120 | 0.0183 | 0.1933 | 0.7897 | 0.2173 | 0.1837 | 0.0459 | 0.0099 | 0.3655 | 5 |
| rs17790811 | SNV | 0.0076 | 0.0139 | 0.0085 | 0.0967 | 0.0098 | 0.1409 | 0.1207 | 0.0900 | 0.0078 | 0.1618 | 0.0533 | 0.1360 | 5 |
| rs1048661 | SNV | 0.3382 | 0.1764 | 0.5164 | 0.4138 | 0.4750 | 0.0086 | 0.3799 | 0.0442 | 0.0067 | 0.0063 | 0.0049 | 0.0671 | 5 |
| rs1065381 | SNV | 0.0170 | 0.2866 | 0 0077 | 0.3350 | 0.0885 | 0.3135 | 0.0169 | 0.6904 | 0.0450 | 0.4431 | 0.0250 | 1.0000 | 5 |
| rs381852 | SNV | 0.6680 | 0.0207 | 0.2669 | 0.3347 | 0.2058 | 0.1429 | 0.0008 | 0.1100 | 0.0031 | 0.0520 | 0.0070 | 0.0005 | 5 |
| rs149457868 | SNV | 0.0212 | 0.4166 | 0.0023 | 0.0156 | 0.0107 | 0.1039 | 0.1774 | 0.5705 | 0.0621 | 0.5262 | 0.0330 | 0.2051 | 5 |
| rs132700 | SNV | 0.0666 | 0.1325 | 0.2406 | 0.0406 | 0.0892 | 0.0972 | 0.0189 | 0.2600 | 0.0109 | 0.0508 | 0.0009 | 0.0037 | 5 |
| rs57115249 | SNV | 0.0216 | 0.8942 | 0.0328 | 0.0031 | 0.0058 | 0.1385 | 0.6587 | 0.9499 | 0.0750 | 0.0408 | 0.1073 | 0.2834 | 5 |
| chr2:87423360-87423997 | CNV | 0.0460 | 0.0060 | 0.3253 | 0.0079 | 0.0186 | 0.0933 | 0.3939 | 0.1846 | 0.6918 | 0.0989 | 0.0580 | 0.0436 | 5 |
| rs1051266 | SNV | 0.3559 | 0.3114 | 0.5902 | 0.8833 | 0.8748 | 0.7288 | 0.0327 | 0.0224 | 0.0008 | 0.0409 | 0.1031 | 0.0197 | 5 |
| rs2302858 | SNV | 0.1048 | 0.0335 | 0.3041 | 0.0182 | 0.0242 | 0.3104 | 0.0169 | 0.1736 | 0.0276 | 0.4843 | 0.0705 | 0.1248 | 5 |
| rs45547640 | SNV | 0.0842 | 0.2483 | 0.1394 | 0.0066 | 0.0162 | 0.7525 | 0.2584 | 0.0416 | 0.4920 | 0.0327 | 0.0080 | 0.1643 | 5 |
| rs61754935 | SNV | 0.2008 | 0.0172 | 0 0543 | 0.3096 | 0.3093 | 0.1478 | 0.0284 | 0.0039 | 0.2143 | 0.2304 | 0.0345 | 0.0156 | 5 |
| rs2073214 | SNV | 0.1349 | 0.1487 | 0.0379 | 0.1560 | 0.2636 | 0.2213 | 0.2955 | 0.2708 | 0.0005 | 0.0147 | 0.0051 | 0.0048 | 5 |
| rs6032259 | SNV | 0.0208 | 0.2672 | 0.1550 | 0.0003 | 0.0014 | 0.6564 | 0.0180 | 0.7267 | 0.5468 | 0.4421 | 0.0488 | 0.3481 | 5 |
| rs2274802 | SNV | 0.0072 | 0.0007 | 0.0368 | 0.2363 | 0.1822 | 0.1053 | 0.0319 | 0.0062 | 0.2471 | 0.0649 | 0.3175 | 0.1278 | 5 |
| rs6736922 | SNV | 0.5819 | 0.0206 | 0.5022 | 0.1036 | 0.1435 | 0.0041 | 0.0007 | 0.0008 | 0.8561 | 0.0467 | 0.2620 | 0.0552 | 5 |
| rs76326644 | SNV | 0.0396 | 0.0481 | 0.1121 | 0.1904 | 0.0953 | 0.0229 | 0.0737 | 0.1853 | 0.0053 | 0.0676 | 0.1048 | 0.0105 | 5 |
| rs2257167 | SNV | 0.3235 | 0.3267 | 0.2822 | 0.1794 | 0.1152 | 0.0060 | 0.0149 | 0.6441 | 0.1191 | 0.0391 | 0.0391 | 0.0166 | 5 |
| rs8012505 | SNV | 0.1127 | 0.3125 | 0.0267 | 0.2518 | 0.1839 | 0.0423 | 0.1673 | 0.2359 | 0.3670 | 0.0320 | 0.0024 | 0.0289 | 5 |
| rs3747673 | SNV | 0.0191 | 0.8419 | 0.0241 | 0.0196 | 0.0097 | 0.1348 | 0.6721 | 0.7594 | 0.2832 | 0.1469 | 0.0369 | 0.2846 | 5 |
| rs13336129 | SNV | 0.0071 | 0.0632 | 0.0206 | 0.0797 | 0.0777 | 0.0346 | 0.9922 | 0.2025 | 0.3119 | 0.0177 | 0.0384 | 0.6602 | 5 |
| rs78970023 | SNV | 0 0489 | 0 7956 | 0 0807 | 0 0916 | 0.0950 | 0 0532 | 0 0419 | 0 2102 | 0 1554 | 0 0429 | 0 0167 | 0.0035 | 5 |
| rs866484 | SNV | 0.2251 | 0.2377 | 0.0313 | 0.0773 | 0.0285 | 0.2604 | 0.4096 | 0.5256 | 0.0470 | 0.0524 | 0.0075 | 0.0103 | 5 |
| rs74953707 | SNV | 0.0203 | 0.1523 | 0.0862 | 0.0485 | 0.0036 | 0.3588 | 0.7585 | 0.1873 | 0.0238 | 0.0480 | 0.1515 | 0.5501 | 5 |
| rs76511758 | SNV | 0.0037 | 0.9993 | 0.0861 | 0.0392 | 0.0231 | 0.1523 | 0.9945 | 0.9999 | 0.1076 | 0.0384 | 0.0420 | 0.9984 | 5 |
| rs2865121 | SNV | 0.0099 | 0.0021 | 0.0356 | 0.0172 | 0.0702 | 0.2495 | 0.4387 | 0.0182 | 0.9584 | 0.1906 | 0.3339 | 0.4863 | 5 |
| rs9843784 | SNY | 0.0416 | 0.2405 | 0.0137 | 0.0352 | 0.0459 | 0.0054 | 0.0613 | 0.7515 | 0.2072 | 0.0657 | 0.2193 | 0.0921 | 5 |
| chr1:151371721-151372092 | CNV | 0.0882 | 0.0055 | 0.2328 | 0.0021 | 0.0060 | 0.0793 | 0.5227 | 0.0848 | 0.2571 | 0.0690 | 0.0272 | 0.0179 | 5 |
| rs2347132 | SNV | 0.0120 | 0.0215 | 0.0930 | 0.0175 | 0.0008 | 0.0999 | 0.7335 | 0.0108 | 0.8545 | 0.1135 | 0.0889 | 0.1235 | 5 |
| rs2347131 | SNV | 0.0179 | 0.0367 | 0.0930 | 0.0296 | 0.0026 | 0.0999 | 0.7335 | 0.0108 | 0.8545 | 0.1135 | 0.1215 | 0.1235 | 5 |
| rs3732775 | SNV | 0.0276 | 0.5524 | 0.0535 | 0.0122 | 0.0093 | 0.0348 | 0.6086 | 0.4603 | 0.6415 | 0.0272 | 0.1458 | 0.5316 | 5 |
| rs10401454 | SNV | 0.0086 | 0.1431 | 0.1097 | 0.0123 | 0.0004 | 0.1175 | 0.4782 | 0.5040 | 0.0402 | 0.1068 | 0.0233 | 0.1843 | 5 |
| rs2291533 | SNV | 0.0034 | 0.0318 | 0.0119 | 0.1015 | 0.0599 | 0.0566 | 0.2516 | 0.7471 | 0.1079 | 0.0040 | 0.0017 | 0.3543 | 5 |
| chr1:151337334-151337904 | CNV | 0.0882 | 0.0055 | 0.2328 | 0.0021 | 0.0060 | 0.0793 | 0.5227 | 0.0848 | 0.2571 | 0.0690 | 0.0272 | 0.0179 | 5 |
| rs2274254 | SNV | 0.0087 | 0.1298 | 0.0336 | 0.4014 | 0.2138 | 0.0080 | 0.0193 | 0.1094 | 0.0481 | 0.0509 | 0.1531 | 0.1514 | 5 |
| rs17080319 | SNV | 0.0019 | 0.0060 | 0.0782 | 0.0050 | 0.0030 | 0.1328 | 0.3261 | 0.0429 | 0.0691 | 0.1990 | 0.0775 | 0.1977 | 5 |
| rs4371530 | SNV | 0.4207 | 0.0206 | 0.1070 | 0.1092 | 0.4265 | 0.0028 | 0.0516 | 0.0027 | 0.3796 | 0.0597 | 0.0390 | 0.0016 | 5 |
| rs145477274 | SNV | 0.0180 | 0.4304 | 0.0285 | 0.0590 | 0.0330 | 0.1439 | 0.0014 | 0.9994 | 0.0232 | 0.0563 | 0.0576 | 0.1977 | 5 |
| rs2276932 | SNV | 0.0292 | 0.5962 | 0.0242 | 0.0530 | 0.1312 | 0.0031 | 0.2905 | 0.0539 | 0.3807 | 0.0158 | 0.0747 | 0.0271 | 5 |
| chr1:151377013-151377600 | CNV | 0.0882 | 0.0055 | 0.2328 | 0.0021 | 0.0060 | 0.0793 | 0.5227 | 0.0848 | 0.2571 | 0.0690 | 0.0272 | 0.0179 | 5 |
| rs2281852 | SNV | 0.1674 | 0.1382 | 0.0126 | 0.0250 | 0.0465 | 0.1732 | 0.7993 | 0.6586 | 0.1328 | 0.0782 | 0.0038 | 0.0054 | 5 |
| rs2298804 | SNV | 0.0284 | 0.0788 | 0.0812 | 0.0877 | 0.0596 | 0.0480 | 0.1179 | 0.0023 | 0.0641 | 0.0468 | 0.0120 | 0.2045 | 5 |
| rs2276781 | SNV | 0.0091 | 0.0103 | 0.0790 | 0.0032 | 0.0077 | 0.3784 | 0.9427 | 0.1192 | 0.0178 | 0.1020 | 0.0903 | 0.1098 | 5 |
| rs1047855 | SNV | 0.0261 | 0.0379 | 0.1705 | 0.0782 | 0.0099 | 0.0181 | 0.4859 | 0.2233 | 0.0862 | 0.0218 | 0.1140 | 0.4557 | 5 |
| rs56060894 | SNV | 0.0447 | 0.0797 | 0.2876 | 0.3389 | 0.0377 | 0.5997 | 0.6407 | 0.0141 | 0.0012 | 0.1815 | 0.0143 | 0.0613 | 5 |
| rs2276774 | SNV | 0 0063 | 0 0155 | 0 0580 | 0 0021 | 0 0064 | 0 4752 | 0 8298 | 0 1601 | 0 0269 | 0 1468 | 0 1298 | 0 1475 | 5 |
| rs16973716 | SNV | 0.0017 | 0.0142 | 0.0044 | 0.0323 | 0.0042 | 0.6063 | 0.1703 | 0.8839 | 0.9574 | 0.3861 | 0.1091 | 0.1448 | 5 |
| rs28497577 | SNV | 0.0286 | 0.2475 | 0.6538 | 0.0061 | 0.0017 | 0.3419 | 0.1897 | 0.0672 | 0.0230 | 0.1121 | 0.0103 | 0.1595 | 5 |
| rs782800 | SNV | 0.0029 | 0.8124 | 0.0314 | 0.7379 | 0.5419 | 0.0084 | 0.3633 | 0.8296 | 0.4027 | 0.0049 | 0.0387 | 0.8859 | 5 |
| rs3732496 | SNV | 0.0562 | 0.0311 | 0.1322 | 0.0528 | 0.0480 | 0.1679 | 0.0094 | 0.0172 | 0.2112 | 0.1631 | 0.0392 | 0.9998 | 5 |
| rs12512164 | SNV | 0.0333 | 0.1680 | 0.0052 | 0.0143 | 0.0629 | 0.8255 | 0.8249 | 0.2966 | 0.0039 | 0.6166 | 0.0065 | 0.2904 | 5 |
| rs11538927 | SNV | 0.0233 | 0.0004 | 0.5167 | 0.2234 | 0.4231 | 0.0815 | 0.3166 | 0.0107 | 0.2019 | 0.0643 | 0.0407 | 0.0228 | 5 |
| rs730469 | SNV | 0.1676 | 0.0597 | 0.2837 | 0.6109 | 0.2584 | 0.0016 | 0.0069 | 0.0148 | 0.1467 | 0.0028 | 0.0178 | 0.0942 | 5 |
| rs227787 | SNV | 0.0222 | 0.0028 | 0.0567 | 0.0011 | 0.0022 | 0.5953 | 0.4333 | 0.0096 | 0.0610 | 0.7714 | 0.1692 | 0.0651 | 5 |
| rs2304968 | SNV | 0.0007 | 0.2137 | 0.0357 | 0.2627 | 0.0588 | 0.0491 | 0.4480 | 0.6378 | 0.6394 | 0.0319 | 0.0262 | 0.4778 | 5 |
| rs3850625 | SNV | 0.0199 | 0.1197 | 0.0046 | 0.1691 | 0.0572 | 0.0090 | 0.8176 | 0.0383 | 0.7927 | 0.0616 | 0.0177 | 0.5006 | 5 |
| rs1107413 | SNT | 0.0266 | 0.0934 | 0.3652 | 0.0864 | 0.0263 | 0.0897 | 0.0536 | 0.8172 | 0.0007 | 0.0105 | 0.0063 | 0.2342 | 5 |
| rs2303810 | SNV | 0.0688 | 0.0263 | 0.2283 | 0.0164 | 0.0885 | 0.2610 | 0.4739 | 0.0042 | 0.0453 | 0.0579 | 0.1545 | 0.0046 | 5 |
| chr7:144015382-144015845 | CNV | 0.0116 | 0.9313 | 0.0064 | 0.4260 | 0.3826 | 0.0060 | 0.1156 | 0.1361 | 0.5191 | 0.0153 | 0.0343 | 0.8967 | 5 |
| rs12872889 | SNV | 0.1911 | 0.2079 | 0.0352 | 0.4245 | 0.6695 | 0.1589 | 0.3043 | 0.0623 | 0.0106 | 0.0195 | 0.0056 | 0.0108 | 5 |
| WBC | LAB | 0.0301 | 0.2021 | 0.1748 | 0.0278 | 0.0025 | 0.3638 | 0.0417 | 0.4629 | 0.0520 | 0.2169 | 0.0430 | 0.1680 | 5 |
| rs151250620 | SNV | 0.0337 | 0.6162 | 0.1191 | 0.1006 | 0.1314 | 0.0080 | 0.0501 | 0.2276 | 0.2041 | 0.0050 | 0.0076 | 0.0036 | 5 |
| rs79812711 | SNV | 0.3022 | 0.3237 | 0.7854 | 0.4268 | 0.3642 | 0.0048 | 0.0233 | 0.9319 | 0.0394 | 0 0083 | 0.0195 | 0.3866 | 5 |
| rs3736822 | SNV | 0.0398 | 0.0048 | 0.6402 | 0.1140 | 0.0355 | 0.0812 | 0.3613 | 0.0053 | 0.2494 | 0.2395 | 0.1426 | 0.0160 | 5 |
| rs143121480 | SNV | 0.0333 | 0.0116 | 0.0862 | 0.3307 | 0.2837 | 0.3730 | 0.0237 | 0.0645 | 0.0031 | 0.1406 | 0.1045 | 0.0183 | 5 |
| rs140382957 | SNV | 0.1010 | 0.0336 | 0.3839 | 0.1233 | 0.1903 | 0.0787 | 0.0493 | 0.0057 | 0.0483 | 0.0690 | 0.0165 | 0.2230 | 5 |
| rs6459165 | SNV | 0.2707 | 0.0589 | 0.1982 | 0.3694 | 0.5934 | 0.0295 | 0.1214 | 0.4731 | 0.0148 | 0.0348 | 0.0431 | 0.0005 | 3 |
| rs11051266 | SNV | 0.0074 | 0.8771 | 0.0027 | 0.0105 | 0.0216 | 0.5060 | 0.4331 | 0.6946 | 0.3024 | 0.1050 | 0.0344 | 0.1227 | 5 |
| rs77474006 | SNV | 0.0040 | 0.0651 | 0.0064 | 0.1627 | 0.1255 | 0.0829 | 0.0613 | 0.0422 | 0.3126 | 0.0069 | 0.0396 | 0.8456 | 5 |
| rs597570 | SNV | 0.0109 | 0.0518 | 0.0541 | 0.1286 | 0.0492 | 0.1160 | 0.2911 | 0.7138 | 0.0001 | 0.0370 | 0.0018 | 0.0781 | 5 |
| rs1024196 | SNV | 0.2707 | 0.0589 | 0.1982 | 0.3694 | 0.5934 | 0.0295 | 0.1214 | 0.4731 | 0.0148 | 0.0348 | 0.0431 | 0.0005 | 5 |
| rs10838851 | SNV | 0.8015 | 0.2350 | 0.1500 | 0.7915 | 0.9324 | 0.0224 | 0.0019 | 0.5151 | 0.0335 | 0.0746 | 0.0224 | 0.0142 | 5 |
| rs1766848 | SNV | 0.0717 | 0.0192 | 0.4634 | 0.3458 | 0.2659 | 0.0060 | 0.2754 | 0.0001 | 0.3725 | 0.0194 | 0.0336 | 0.5413 | 5 |
| rs12794960 | SNV | 0.1175 | 0.0207 | 0.1364 | 0.2030 | 0.0822 | 0.0726 | 0.0004 | 0.8582 | 0.0087 | 0.1555 | 0.0059 | 0.0261 | 5 |
| rs117866676 | SNV | 0.0019 | 0.3987 | 0.0577 | 0.0236 | 0.0042 | 0.0027 | 0.5593 | 0.2536 | 0.4204 | 0.0029 | 0.0532 | 0.1721 | 5 |
| rs2288672 | SNV | 0 8198 | 0 0027 | 0 2861 | 0 9663 | 0 2632 | 0 0420 | 0 3363 | 0 0618 | 0 0150 | 0 2213 | 0 0377 | 0 0108 | 5 |
| rs72655972 | SNV | 0.0218 | 0.0279 | 0.2382 | 0.5951 | 0.4175 | 0.0034 | 0.2195 | 0.0099 | 0.1633 | 0 0016 | 0.1359 | 0.6139 | 5 |
| rs10783816 | SNV | 0.2086 | 0.2718 | 0.3314 | 0.3479 | 0.5846 | 0.1083 | 0.0433 | 0.0364 | 0.0019 | 0.0125 | 0.0042 | 0.3327 | 5 |
| rs1948516 | SNV | 0.0179 | 0.0502 | 0.0112 | 0.0908 | 0.1308 | 0.2561 | 0.0057 | 0.1014 | 0.0093 | 0.0180 | 0.1002 | 0.3091 | 5 |
| rs2302757 | SNV | 0.0110 | 0.0005 | 0.5046 | 0.0262 | 0.0238 | 0.3739 | 0.0664 | 0.0071 | 0.3374 | 0.0620 | 0.0995 | 0.7262 | 5 |
| rs116981543 | SNV | 0.0051 | 0.1975 | 0.0233 | 0.0436 | 0.0533 | 0.0443 | 0.0385 | 0.3116 | 0.8999 | 0.2065 | 0.1385 | 0.9321 | 5 |
| rs9507425 | SNV | 0.0158 | 0.4073 | 0.0225 | 0.4798 | 0.3779 | 0.0019 | 0.0530 | 0.6515 | 0.2289 | 0.0012 | 0.0106 | 0.0551 | 5 |
| rs4534 | SNV | 0.0024 | 0.1083 | 0.0358 | 0.1295 | 0.0578 | 0.0213 | 0.9593 | 0.1466 | 0.8334 | 0.0165 | 0.0443 | 0.6418 | 5 |
| rs147741170 | SNV | 0.0125 | 0.0097 | 0.1609 | 0.6011 | 0.4585 | 0.5589 | 0.0252 | 0.0169 | 0.0186 | 0.2357 | 0.1069 | 0.4125 | 5 |
| Abs_Lymphocyte | LAB | 0.0077 | 0.4090 | 0.0562 | 0.3694 | 0.2321 | 0.0047 | 0.5749 | 0.6584 | 0.0148 | 0.0007 | 0.0079 | 0.1955 | 5 |
| rs2303757 | SNV | 0.0134 | 0.1682 | 0.0152 | 0.1587 | 0.0620 | 0.0069 | 0.1568 | 0.9996 | 0.1004 | 0.0107 | 0.0788 | 0 8201 | 4 |
| chr9:135946828-135947422 | CNV | 0.0262 | 0.5236 | 0.0472 | 0.5430 | 0.1785 | 0.1076 | 0.1458 | 0.3252 | 0.3457 | 0.0263 | 0.0097 | 0.5866 | 4 |
| rs12107252 | SNV | 0.0336 | 0.0103 | 0.1247 | 0.0076 | 0.0454 | 0.2648 | 0.4799 | 0.0709 | 0.2032 | 0.6623 | 0.2228 | 0.1660 | 4 |
| rs4858871 | SNV | 0.1551 | 0.0080 | 0.0100 | 0.2072 | 0.3209 | 0.5958 | 0.9417 | 0.0108 | 0.1631 | 0.9364 | 0.2150 | 0.0237 | 4 |
| chr19:54804071-54804408 | CNV | 0.4517 | 0.1058 | 0.0896 | 0.0589 | 0.0424 | 0.2989 | 0.0430 | 0.9753 | 0.0711 | 0.5031 | 0.0335 | 0.0096 | 4 |
| rs17849654 | SNV | 0.0629 | 0.0917 | 0.1504 | 0.0819 | 0.1023 | 0.0307 | 0.4817 | 0.8473 | 0.0032 | 0.0384 | 0.3645 | 0.0124 | 4 |
| rs9617066 | SNV | 0.0138 | 0.2410 | 0.6405 | 0.0273 | 0.0100 | 0.3151 | 0.4405 | 0.0294 | 0.9544 | 0.1232 | 0.6442 | 0.5149 | 4 |
| rs10402252 | SNV | 0.0225 | 0.0103 | 0.0133 | 0.2864 | 0.2208 | 0.2549 | 0.1574 | 0.2667 | 0.0166 | 0.1726 | 0.1038 | 0.0755 | 4 |
| rs149641101 | SNV | 0.6155 | 0.0071 | 0.6440 | 0.8786 | 0.8937 | 0.3375 | 0.3607 | 0.0260 | 0.0058 | 0.8359 | 0.3232 | 0.0390 | 4 |
| rs3745489 | SNV | 0.3006 | 0.4665 | 0.1938 | 0.6758 | 0.4374 | 0.0473 | 0.7175 | 0.8692 | 0.0004 | 0.2510 | 0.0096 | 0.0424 | 4 |
| rs2003783 | SNV | 0.0083 | 0 2709 | 0.0028 | 0.3513 | 0.0544 | 0.0570 | 0.1325 | 0.1942 | 0.1145 | 0.0323 | 0.0097 | 0 5665 | 4 |
| rs12460779 | SNV | 0.0378 | 0.5629 | 0.0782 | 0.3030 | 0.2190 | 0.0204 | 0.1778 | 0.4684 | 0.1065 | 0.0086 | 0.0081 | 0.2210 | 4 |
| rs76305623 | SNV | 0.0390 | 0.6528 | 0.0908 | 0.5815 | 0.2044 | 0.1056 | 0.0511 | 0.9097 | 0.0106 | 0.1290 | 0.0211 | 0.0268 | 4 |
| rs115964471 | SNV | 0.1127 | 0.0026 | 0.1950 | 0.7630 | 0.5172 | 0.0688 | 1.0000 | 0.0074 | 0.0146 | 0.0553 | 0.0338 | 0.0948 | 4 |
| rs11606506 | SNV | 0.2950 | 0.0281 | 0.2046 | 0.3766 | 0.3376 | 0.2991 | 0.3649 | 0.0393 | 0.3564 | 0.1170 | 0.0085 | 0.0034 | 4 |
| rs862708 | SNV | 0 1386 | 0 0486 | 0 0252 | 0 2714 | 0 1279 | 0 2712 | 0 2855 | 0 6614 | 0 1996 | 0 0416 | 0 0077 | 0 0801 | 4 |
| rs77803411 | SNV | 0.0314 | 0.0238 | 0.1184 | 0.1341 | 0.0041 | 0.2190 | 0.2155 | 0.0300 | 0.1674 | 0.1562 | 0.1361 | 0 7040 | 4 |
| rs2285906 | SNV | 0.0252 | 0.1934 | 0.2704 | 0.0309 | 0.0013 | 0.1611 | 0.3812 | 0.5988 | 0.0988 | 0.1482 | 0.0349 | 0.3967 | 4 |
| rs2304261 | SNV | 0.0208 | 0.1154 | 0.4237 | 0.0058 | 0.0025 | 0.0770 | 0.4603 | 0.2256 | 0.1791 | 0.0483 | 0.0687 | 0.2738 | 4 |
| rs10899750 | SNV | 0.5898 | 0.0024 | 0.8697 | 0.3526 | 0.9940 | 0.2369 | 0.0077 | 0.0163 | 0.0029 | 0.2884 | 0.1697 | 0.1200 | 4 |
| rs4253301 | SNV | 0.0272 | 0.8117 | 0.0348 | 0.0831 | 0.0109 | 0.2185 | 0.9505 | 0.8232 | 0.2533 | 0.0330 | 0.1644 | 0.9625 | 4 |
| rs7949157 | SNV | 0.8752 | 0.1303 | 0.1884 | 0.1538 | 0.1937 | 0.1066 | 0.0040 | 0.0056 | 0.0332 | 0.0782 | 0.0363 | 0.1295 | 4 |
| chr1:152586494-152586889 | CNV | 0.0049 | 0.1104 | 0.0204 | 0.0061 | 0.0060 | 0.4160 | 0.8379 | 0.4646 | 0.1265 | 0.1225 | 0.0672 | 0.6841 | 4 |
| chr1:152586094-152586493 | CNV | 0.0037 | 0.1104 | 0.0131 | 0.0061 | 0.0060 | 0.4160 | 0.8379 | 0.4646 | 0.1265 | 0.1225 | 0.0672 | 0.6841 | 4 |
| rs9913145 | SNV | 0.0601 | 0.9959 | 0.2422 | 0.0168 | 0.0106 | 0.0186 | 0.2642 | 0.7835 | 0.2913 | 0.0079 | 0.0866 | 0.2812 | 4 |
| chr1:152573024-152573644 | CNV | 0.0081 | 0.1889 | 0.0253 | 0.0106 | 0.0089 | 0.5861 | 0.3291 | 0.3309 | 0.1530 | 0.1512 | 0.1379 | 0.8504 | 4 |
| rs557491 | SNV | 0.2555 | 0.0105 | 0.0533 | 0.3842 | 0.0719 | 0.1786 | 0.0025 | 0.0684 | 0.0076 | 0.3179 | 0.0277 | 0.1314 | 4 |
| rs3803784 | SNV | 0.0741 | 0.1051 | 0.0411 | 0.0177 | 0.0716 | 0.0104 | 0.1735 | 0.1469 | 0.9052 | 0.1037 | 0.0069 | 0.0521 | 4 |
| rs1042522 | SNV | 0.4057 | 0.3394 | 0.3581 | 0.5780 | 0.6777 | 0.0594 | 0.0005 | 0.1703 | 0.0395 | 0.0310 | 0.0975 | 0.0004 | 4 |
| chr4:69342831-69343615 | CNV | 0.0077 | 0 3442 | 0.0173 | 0.0212 | 0.0178 | 0.1929 | 0.1727 | 0.7297 | 0.1904 | 0.4048 | 0.3710 | 0.2907 | 4 |
| rs7503604 | SNV | 0.0002 | 0.0732 | 0.0160 | 0.0509 | 0.0300 | 0.2596 | 0.5489 | 0.0559 | 0.6256 | 0.0462 | 0.0687 | 0.6491 | 4 |
| rs77833427 | SNV | 0.0334 | 0.4140 | 0.0534 | 0.0421 | 0.1482 | 0.5238 | 0.1724 | 0.7653 | 0.0002 | 0.1856 | 0.0289 | 0.1977 | 4 |
| rs34241745 | SNV | 0.0361 | 0.0667 | 0.0452 | 0.2001 | 0.0449 | 0.7959 | 0.2018 | 0.0970 | 0.0066 | 0.5617 | 0.0917 | 0.1630 | 4 |
| rs118176767 | SNV | 0.0477 | 0.0083 | 0.7907 | 0.3211 | 0.1745 | 0.0228 | 0.6208 | 0.0102 | 0.0677 | 0.0554 | 0.0554 | 0.0765 | 4 |
| rs227368 | SNV | 0.0020 | 0.6832 | 0.0868 | 0.0613 | 0.0374 | 0.0394 | 0.9040 | 0.0843 | 0.9237 | 0.0124 | 0.2315 | 0.1412 | 4 |
| rs33944211 | SNV | 0.0308 | 0.3386 | 0.0274 | 0.0180 | 0.0039 | 0.9990 | 0.0868 | 0.4797 | 0.3372 | 0.4255 | 0.0791 | 0.9136 | 4 |
| rs181750881 | SNV | 0.0429 | 0.0117 | 0.0600 | 0.0854 | 0.0233 | 0.1815 | 0.5862 | 0.0086 | 0.5579 | 0.2432 | 0.0888 | 0.0513 | 4 |
| rs141974918 | SNV | 0.0093 | 0.2845 | 0.0326 | 0.0214 | 0.0100 | 0.0748 | 0.1880 | 0.0856 | 0.1916 | 0.0819 | 0.0571 | 0.3381 | 7 |
| rs2277969 | SNV | 0.0314 | 0.8752 | 0.0207 | 0.0047 | 0.0083 | 0.8901 | 0.8918 | 0.3528 | 0.6741 | 0.8959 | 0.2469 | 0.5291 | 4 |
| rs9898 | SNV | 0.4932 | 0.0035 | 0.1651 | 0.7514 | 0.5751 | 0.7537 | 0.1480 | 0.0017 | 0.0385 | 0.6109 | 0.0475 | 0.2549 | 4 |
| rs8135914 | SNV | 0.0105 | 0.1796 | 0.0365 | 0.0036 | 0.0018 | 0.3306 | 0.5355 | 0.1355 | 0.8369 | 0.1429 | 0.1909 | 0.1725 | 4 |
| rs2839716 | SNV | 0.1377 | 0.5449 | 0.5181 | 0.0202 | 0.0053 | 0.3026 | 0.9162 | 0.2858 | 0.0490 | 0.1630 | 0.1545 | 0.0212 | 4 |
| Albumin | LAB | 0.0005 | 0 3826 | 0.0464 | 0.4239 | 0.1578 | 0.1343 | 0.9221 | 0.9836 | 0.0635 | 0.0273 | 0.0334 | 0 8739 | 4 |
| rs2227313 | SNY | 0.2185 | 0.1382 | 0.0257 | 0.0250 | 0.0669 | 0.2404 | 0.7993 | 0.6586 | 0.1328 | 0.1050 | 0.0055 | 0.0054 | 4 |
| rs3814171 | SNV | 0.1546 | 0.2986 | 0.4442 | 0.0413 | 0.1553 | 0.2538 | 0.0175 | 0.3738 | 0.0095 | 0.1058 | 0.0195 | 0.4344 | 4 |
| rs3781411 | SNY | 0.0038 | 0.1088 | 0.1497 | 0.0044 | 0.0124 | 0.2024 | 0.5864 | 0.0076 | 0.7254 | 0.1009 | 0.3251 | 0.6095 | 4 |
| rs12735670 | SNV | 0.1772 | 0.0580 | 0.1181 | 0.0346 | 0.0148 | 0.3422 | 0.0858 | 0.2904 | 0.0497 | 0.2149 | 0.0685 | 0.0009 | 4 |
| rs7526812 | SNV | 0.0655 | 0.1472 | 0.0753 | 0.3670 | 0.2946 | 0.0070 | 0.0827 | 0.4229 | 0.2786 | 0.0349 | 0.0148 | 0.0057 | 4 |
| rs11196686 | SNV | 0.0443 | 0.0271 | 0.0444 | 0.4767 | 0.3004 | 0.8748 | 0.1248 | 0.1093 | 0.2988 | 0.5117 | 0.0103 | 0.4964 | 4 |
| rs1649292 | SNV | 0.0197 | 0.8838 | 0.0025 | 0.0251 | 0.0237 | 0.0887 | 0.8027 | 0.9509 | 0.5756 | 0.0976 | 0.1507 | 0.4015 | 4 |
| rs117677354 | SNV | 0.0324 | 0.3347 | 0.1107 | 0.1853 | 0.1484 | 0.3270 | 0.0010 | 0.5352 | 0.0090 | 0.0430 | 0.1222 | 0.3725 | 4 |
| rs6757448 | SNV | 0.5552 | 0.1106 | 0.6420 | 0.2787 | 0.3008 | 0.0011 | 0.0515 | 0.0013 | 0.0526 | 0.0035 | 0.0924 | 0.0032 | 4 |
| rs80140823 | SNV | 0 0404 | 0 0412 | 0 2890 | 0 0127 | 0 0066 | 0 4095 | 0 3215 | 0 2545 | 0 7754 | 0.8249 | 0.7036 | 0 7255 | 4 |
| rs142580477 | SNV | 0.0547 | 0.1142 | 0.0298 | 0.1843 | 0.3325 | 0.5079 | 0.0221 | 0.4686 | 0.0078 | 0.0617 | 0.0383 | 0.5599 | 4 |
| rs117512489 | SNV | 0.0842 | 0.6319 | 0.1880 | 0.0036 | 0.0203 | 0.8088 | 0.2129 | 0.8830 | 0.0335 | 0.3908 | 0.0305 | 0.6798 | 4 |
| rs145952310 | SNV | 0.0773 | 0.0406 | 0.0438 | 0.1176 | 0.3027 | 0.0597 | 0.0705 | 0.0015 | 0.1852 | 0.0938 | 0.0350 | 0.2031 | 4 |
| rs12686794 | SNV | 0.0267 | 0.9013 | 0.0056 | 0.1104 | 0.0933 | 0.0224 | 0.1893 | 0.8325 | 0.2726 | 0.0390 | 0.0930 | 0.1655 | 4 |
| chr6:32005948-32006709 | CNV | 0.0356 | 0 0773 | 0.1739 | 0.0746 | 0.0523 | 0.3434 | 0.4049 | 0.6301 | 0.0194 | 0.1269 | 0.0082 | 0 0367 | 4 |
| rs1805127 | SNV | 0.1457 | 0.1443 | 0.1350 | 0.3617 | 0.2068 | 0.0230 | 0.1305 | 0.0235 | 0.0755 | 0.0123 | 0.1214 | 0.0029 | 4 |
| rs3746866 | SNV | 0.0595 | 0.0047 | 0.0155 | 0.1406 | 0.2030 | 0.0786 | 0.0026 | 0.1908 | 0.0548 | 0.5566 | 0.1138 | 0.0025 | 4 |
| rs35044862 | SNV | 0.0625 | 0.1205 | 0.0981 | 0.2354 | 0.0729 | 0.0013 | 0.2439 | 0.0893 | 0.0544 | 0.0064 | 0.0214 | 0.0001 | 4 |
| rs3793395 | SNV | 0.0791 | 0.0047 | 0.1556 | 0.0611 | 0.0363 | 0.0483 | 0.0984 | 0.0068 | 0.0897 | 0.2301 | 0.0631 | 0.0968 | 4 |
| rs2236358 | SNV | 0.0066 | 0.9593 | 0.0008 | 0.6119 | 0.4649 | 0.0446 | 0.2385 | 0.4316 | 0.1498 | 0.0063 | 0.0624 | 0.7548 | 4 |
| rs139767460 | SNV | 0.0917 | 0.0046 | 0.4535 | 0.3861 | 0.4753 | 0.0150 | 0.8464 | 0.0086 | 0.1137 | 0.0279 | 0.6174 | 0.1973 | 4 |
| rs2295950 | SNV | 0.0315 | 0.0166 | 0.3064 | 0.0022 | 0.0045 | 0.4453 | 0.9064 | 0.2036 | 0.7694 | 0.9400 | 0.1580 | 0.1790 | 4 |
| rs2305008 | SNV | 0 0390 | 0.0151 | 0 9785 | 0.0033 | 0.0005 | 0 3729 | 0.1563 | 0.0854 | 0.1087 | 0 2277 | 0 0589 | 0.3430 | 4 |
| PD1 | LAB | 0.0143 | 0.0281 | 0.0134 | 0.1634 | 0.0876 | 0.1502 | 0.4985 | 0.4372 | 0.0625 | 0.1648 | 0.0058 | 0.0636 | 4 |
| rs41521547 | SNV | 0.0431 | 0.0060 | 0.7375 | 0.0651 | 0.0127 | 0.2772 | 0.3421 | 0.0053 | 0.0885 | 0.2240 | 0.0628 | 0.5069 | 4 |
| rs17852813 | SNV | 0.0702 | 0.9917 | 0.1282 | 0.0176 | 0.0544 | 0.0400 | 0.4919 | 0.3319 | 0.5228 | 0.0078 | 0.0244 | 0.7444 | 4 |
| rs3737786 | SNV | 0.0204 | 0.1604 | 0.0010 | 0.4813 | 0.1530 | 0.0161 | 0.2251 | 0.1173 | 0.1798 | 0.0519 | 0.0332 | 0.4032 | 4 |
| rs140081 | SNV | 0.0710 | 0.0881 | 0.1074 | 0.6211 | 0.4977 | 0.3186 | 0.1861 | 0.5166 | 0.0427 | 0.0349 | 0.0019 | 0.0197 | 4 |
| rs10489177 | SNV | 0.0594 | 0.0666 | 0.0990 | 0.7102 | 0.5196 | 0.0048 | 0.2240 | 0.2887 | 0.0036 | 0.0069 | 0.0337 | 0.0783 | 4 |
| rs78245253 | SNV | 0.0422 | 0.0342 | 0.0057 | 0.1129 | 0.1095 | 0.4341 | 0.1946 | 0.3085 | 0.1614 | 0.2316 | 0.0418 | 0.0575 | 4 |
| rs12288277 | SNV | 0.0340 | 0.0105 | 0.1625 | 0.0039 | 0.0202 | 0.2795 | 0.3438 | 0.3678 | 0.2273 | 0.6900 | 0.0765 | 0.0800 | 4 |
| rs3748222 | SNV | 0.0143 | 0.0021 | 0.1286 | 0.1997 | 0.1682 | 0.2229 | 0.9711 | 0.0579 | 0.0054 | 0.2342 | 0.0219 | 0.0697 | 4 |
| rs2017252 | SNV | 0.4032 | 0.0200 | 0.4184 | 0.2820 | 0.4062 | 0.0082 | 0.4686 | 0.3932 | 0.5838 | 0.0666 | 0.0485 | 0.0332 | 4 |
| chr17:66265434-66265784 | CNV | 0.0296 | 0.4198 | 0.0800 | 0.1123 | 0.0241 | 0.0071 | 0.9971 | 0.2648 | 0.3278 | 0.0182 | 0.0555 | 0.1072 | 4 |
| rs75570293 | SNV | 0.0208 | 0.0272 | 0.0941 | 0.0256 | 0.0089 | 0.2399 | 0.9750 | 0.1128 | 0.6225 | 0.5126 | 0.3332 | 0.3374 | 4 |
| rs2303398 | SNV | 0.0772 | 0.0288 | 0.9206 | 0.6169 | 0.3645 | 0.0542 | 0.1342 | 0.0219 | 0.0013 | 0.0865 | 0.2145 | 0.0044 | 4 |
| rs2135720 | SNV | 0.0016 | 0.1168 | 0.2293 | 0.1468 | 0.1183 | 0.0014 | 0.8577 | 0.2001 | 0.4620 | 0.0167 | 0.0289 | 0.0682 | 4 |
| rs137969620 | SNV | 0.0574 | 0.0019 | 0.5681 | 0.1293 | 0.2792 | 0.2712 | 0.0042 | 0.0023 | 0.8506 | 0.1404 | 0.0425 | 0.1025 | 4 |
| rs1209750 | SNV | 0.2191 | 0.3928 | 0.0539 | 0.6250 | 0.2891 | 0.0456 | 0.1692 | 0.6132 | 0.0683 | 0.0038 | 0.0374 | 0.0321 | 7 |
| rs71644745 | SNV | 0.0082 | 0.6560 | 0.1636 | 0.0586 | 0.1028 | 0.0036 | 0.1160 | 0.5407 | 0.0578 | 0.0047 | 0.0307 | 0.5100 | 4 |
| rs7207403 | SNV | 0.0519 | 0.0318 | 0.4358 | 0.3253 | 0.1805 | 0.4182 | 0.0392 | 0.0917 | 0.1628 | 0.2566 | 0.0083 | 0.0487 | 4 |
| rs58830807 | SNV | 0.1718 | 0.0489 | 0.2408 | 0.0412 | 0.0077 | 0.0169 | 0.7031 | 0.2048 | 0.1263 | 0.1523 | 0.0561 | 0.0609 | 4 |
| rs117501524 | SNV | 0.0782 | 0.1606 | 0.0895 | 0.2400 | 0.1879 | 0.0056 | 0.0154 | 0.1898 | 0.1530 | 0.0175 | 0.0125 | 0.2441 | 4 |
| rs1318648 | SNV | 0.0003 | 0.3878 | 0.0219 | 0.0357 | 0.0130 | 0.3127 | 0.9976 | 0.7338 | 0.7890 | 0.0751 | 0.0645 | 0.9058 | 4 |
| rs80200454 | SNV | 0.3454 | 0.4281 | 0.5483 | 0.3885 | 0.6135 | 0.0098 | 0.3751 | 0.7558 | 0.1069 | 0.0059 | 0.0077 | 0.0435 | 4 |
| rs2277382 | SNV | 0.0120 | 0.0725 | 0.0065 | 0.2119 | 0.3221 | 0.0133 | 0.5478 | 0.2846 | 0.2270 | 0.0283 | 0.1256 | 0.0931 | 4 |
| rs12648 | SNV | 0.0029 | 0.0595 | 0.2431 | 0.1081 | 0.1100 | 0.0228 | 0.1330 | 0.1596 | 0.0660 | 0.0076 | 0.0032 | 0.1231 | 4 |
| rs1550526 | SNV | 0.0068 | 0.3244 | 0.1685 | 0.0050 | 0.0090 | 0.0456 | 0.4411 | 0.7009 | 0.7903 | 0.2256 | 0.1691 | 0.2961 | 4 |
| rs2857663 | SNV | 0.0253 | 0.0722 | 0.5484 | 0.0049 | 0.0246 | 0.1024 | 0.8551 | 0.6509 | 0.0244 | 0.3177 | 0.1818 | 0.2003 | 4 |
| rs75357093 | SNV | 0.0556 | 0.1552 | 0.0862 | 0.8429 | 0.5627 | 0.0054 | 0.6304 | 0.3956 | 0.0303 | 0.0380 | 0.0133 | 0.1343 | 4 |
| rs75256744 | SNV | 0.0040 | 0.9998 | 0.0136 | 0.0891 | 0.0730 | 0.0405 | 0.2865 | 1.0000 | 0.0728 | 0.0638 | 0.0264 | 1.0000 | 4 |
| rs1800378 | SNV | 0.6296 | 0.0043 | 0.4427 | 0.2023 | 0.4066 | 0.1255 | 0.0130 | 0.0276 | 0.1607 | 0.7267 | 0.3302 | 0.0087 | 4 |
| rs735295 | SNV | 0.0095 | 0.0514 | 0.0464 | 0.0040 | 0.0011 | 0.3311 | 0.8677 | 0.3656 | 0.7705 | 0.2199 | 0.0562 | 0.8971 | 4 |
| rs11753513 | SNV | 0.0590 | 0.5316 | 0.0390 | 0.6745 | 0.3968 | 0.0155 | 0.9559 | 0.6359 | 0.9080 | 0.0081 | 0.0435 | 0.8231 | 4 |
| rs2233434 | SNV | 0.0249 | 0.0553 | 0.1200 | 0.1171 | 0.2259 | 0.0000 | 0.2461 | 0.1345 | 0.7187 | 0.0008 | 0.0093 | 0.0765 | 4 |
| rs10831519 | SNV | 0.0037 | 0.1783 | 0.0196 | 0.4187 | 0.7294 | 0.0049 | 0.3167 | 0.7427 | 0.5054 | 0.0093 | 0.1624 | 0.3919 | 4 |
| rs1126617 | SNV | 0.0294 | 0.4444 | 0.2301 | 0.0068 | 0.0056 | 0.0719 | 0.5179 | 0.2309 | 0.0509 | 0.1713 | 0.0392 | 0.5343 | 4 |
| rs137874988 | SNV | 0.0684 | 0.6650 | 0.0314 | 0.0097 | 0.0199 | 0.3271 | 0.9990 | 1.0000 | 0.5979 | 0.0493 | 0.0596 | 0.0526 | 4 |
| rs2077412 | SNV | 0.0009 | 0.2204 | 0.0012 | 0.1184 | 0.0143 | 0.2911 | 0.3356 | 0.6201 | 0.1018 | 0.0553 | 0.0153 | 0.0943 | 4 |
| rs1059491 | SNV | 0.0284 | 0.1563 | 0.2829 | 0.4756 | 0.1029 | 0.0046 | 0.8598 | 0.3464 | 0.0362 | 0.0144 | 0.1530 | 0.1406 | 4 |
| rs180743 | SNV | 0.0873 | 0.2049 | 0.4119 | 0.5080 | 0.1786 | 0.0047 | 0.4923 | 0.6789 | 0.0294 | 0.0275 | 0.1450 | 0.0401 | 4 |
| rs1044498 | SNV | 0.1283 | 0.0352 | 0.6242 | 0.0273 | 0.0695 | 0.6479 | 0.7858 | 0.1486 | 0.0094 | 0.2878 | 0.0935 | 0.0294 | 4 |
| rs117416619 | SNV | 0.0372 | 0.1015 | 0.8505 | 0.3134 | 0.0693 | 0.0092 | 0.0560 | 0.3168 | 0.0642 | 0.0041 | 0.0936 | 0.0275 | 4 |
| rs78180055 | SNV | 0.2179 | 0.0019 | 0.4293 | 0.1020 | 0.2771 | 0.0349 | 0.2252 | 0.0545 | 0.0465 | 0.3116 | 0.1547 | 0.0089 | 4 |
| rs3823111 | SNV | 0.0189 | 0.0833 | 0.0278 | 0.0865 | 0.0559 | 0.0519 | 0.0062 | 0.0736 | 0.1248 | 0.0984 | 0.0080 | 0.3052 | 4 |
| rs3736352 | SNV | 0.5364 | 0.2639 | 0.9743 | 0.0049 | 0.0303 | 0.0486 | 0.4104 | 0.5113 | 0.1388 | 0.2283 | 0.1074 | 0.0409 | 4 |
| rs2070724 | SNV | 0.0098 | 0.1856 | 0.0606 | 0.0292 | 0.0543 | 0.0004 | 0.3638 | 0.2523 | 0.1389 | 0.0008 | 0.1126 | 0.9896 | 4 |
| rs7997 | SNV | 0.2594 | 0.4213 | 0.3403 | 0.3278 | 0.2398 | 0.0206 | 0.0637 | 0.9532 | 0.0206 | 0.0043 | 0.0918 | 0.0134 | 4 |
| rs34899 | SNV | 0.5431 | 0.0087 | 0.0609 | 0.1651 | 0.3013 | 0.4338 | 0.0712 | 0.0850 | 0.0240 | 0.5726 | 0.0217 | 0.0004 | 4 |
| rs20541 | SNV | 0.0012 | 0.0292 | 0.2108 | 0.0260 | 0.0203 | 0.9007 | 0.7707 | 0.7479 | 0.1721 | 0.2585 | 0.4525 | 0.3137 | 4 |
| rs3730089 | SNV | 0.0209 | 0.6063 | 0.0106 | 0.0818 | 0.0569 | 0.1391 | 0.4415 | 0.2101 | 0.0622 | 0.0096 | 0.0089 | 0.4192 | 4 |
| rs2230524 | SNY | 0.0491 | 0.1064 | 0.0004 | 0.7359 | 0.7884 | 0.2398 | 0.0248 | 0.2232 | 0.6697 | 0.3885 | 0.0336 | 0.9313 | 4 |
| rs77732988 | SNV | 0.0352 | 0.0028 | 0.1620 | 0.7161 | 0.7596 | 0.1287 | 0.1153 | 0.0005 | 0.4444 | 0.2732 | 0.0196 | 0.3093 | 4 |
| rs10901333 | SNV | 0.3462 | 0.5630 | 0.0935 | 0.2870 | 0.9053 | 0.0230 | 0.1860 | 0.3330 | 0.2792 | 0.0030 | 0.0083 | 0.0433 | 4 |
| rs10054110 | SNV | 0.0338 | 0.2722 | 0.7019 | 0.0883 | 0.1971 | 0.0012 | 0.0705 | 0.1999 | 0.1404 | 0.0005 | 0.0030 | 0.0779 | 4 |
| rs3765310 | SNV | 0.0191 | 0.2943 | 0.0447 | 0.0068 | 0.0043 | 0.1200 | 0.5202 | 0.8100 | 0.1271 | 0.2506 | 0.1107 | 0.1091 | 4 |
| rs12441101 | SNV | 0.0043 | 0.4828 | 0.0505 | 0.0018 | 0.0146 | 0.5015 | 0.0164 | 0.1288 | 0.1164 | 0.1061 | 0.1354 | 0.7470 | 4 |
| rs1714987 | SNV | 0.2161 | 0.0045 | 0.8430 | 0.5109 | 0.2988 | 0.0108 | 0.0542 | 0.1065 | 0.1247 | 0.0284 | 0.0834 | 0.0077 | 4 |
| rs7744845 | SNV | 0.0571 | 0.0465 | 0.6827 | 0.1706 | 0.3637 | 0.0526 | 0.4468 | 0.4116 | 0.0268 | 0.0068 | 0.0208 | 0.1564 | 4 |
| rs9358856 | SNV | 0.0043 | 0.3366 | 0.0807 | 0.0199 | 0.0077 | 0.0662 | 0.9759 | 0.3910 | 0.4116 | 0.0312 | 0.0619 | 0.5675 | 4 |
| rs7123133 | SNV | 0.0462 | 0.2551 | 0.4319 | 0.0013 | 0.0017 | 0.3854 | 0.3219 | 0.0364 | 0.9523 | 0.2445 | 0.3684 | 0.8008 | 4 |
| rs1805097 | SNV | 0.1574 | 0.0786 | 0.5386 | 0.8948 | 0.5883 | 0.0062 | 0.7410 | 0.0035 | 0.0227 | 0.0633 | 0.2352 | 0.0459 | 4 |
| rs11250 | SNV | 0.7066 | 0.5364 | 0.3607 | 0.3250 | 0.4734 | 0.0631 | 0.0325 | 0.1824 | 0.1259 | 0.0070 | 0.0497 | 0.0078 | 4 |
| rs80210207 | SNV | 0.0101 | 0.0764 | 0.0120 | 0.8833 | 0.9326 | 0.0056 | 0.3534 | 0.6043 | 0.1476 | 0.0027 | 0.0576 | 0.4099 | 4 |
| rs117633128 | SNV | 0.0185 | 0.1228 | 0.1290 | 0.2143 | 0.0922 | 0.0752 | 0.0070 | 0.7563 | 0.0082 | 0.0382 | 0.0604 | 0.0853 | 4 |
| rs4371677 | SNV | 0.1709 | 0.0210 | 0.6447 | 0.0185 | 0.0507 | 0.2853 | 0.1137 | 0.0224 | 0.1280 | 0.4071 | 0.2631 | 0.0031 | 4 |
| rs4713668 | SNV | 0.0015 | 0 3210 | 0.0232 | 0.0400 | 0.0326 | 0.1428 | 0.9244 | 0.7568 | 0.4124 | 0.3492 | 0 0642 | 0.5731 | 4 |
| rs10149146 | SNV | 0.0171 | 0.3330 | 0.1823 | 0.3735 | 0.2957 | 0.0076 | 0.6132 | 0.0571 | 0.2156 | 0.0050 | 0.0362 | 0.0832 | 4 |
| rs79471015 | SNV | 0.0110 | 0.0121 | 0.0472 | 0.0700 | 0.0516 | 0.6162 | 0.2485 | 0.0230 | 0.0625 | 0.6049 | 0.1152 | 0.0644 | 4 |
| rs16894240 | SNV | 0.0135 | 0.3672 | 0.1894 | 0.4881 | 0.1503 | 0.0167 | 0.0221 | 0.8102 | 0.6884 | 0.0054 | 0.2392 | 0.6352 | 4 |
| rs9562353 | SNV | 0.0291 | 0.0042 | 0.2233 | 0.0771 | 0.1465 | 0.2474 | 0.1680 | 0.0672 | 0.0178 | 0.0620 | 0.0714 | 0.0108 | 4 |
| rs117484136 | SNV | 0.1209 | 0.1317 | 0.2857 | 0.0827 | 0.1213 | 0.2078 | 0.8876 | 0.0318 | 0.1367 | 0.0290 | 0.0218 | 0.1030 | 3 |
| rs3739128 | SNV | 0.0679 | 0.3756 | 0.2412 | 0.0086 | 0.0071 | 0.2487 | 0.0392 | 0.9663 | 0.2705 | 0.2671 | 0.1973 | 0.2557 | 3 |
| rs1139795 | SNV | 0.1162 | 0.1040 | 0.1117 | 0.6860 | 0.7175 | 0.0739 | 0.3514 | 0.0475 | 0.0143 | 0.2366 | 0.1353 | 0.0057 | 3 |
| chr17:58078320-58078802 | CNV | 0.1079 | 0.1487 | 0.2396 | 0.0751 | 0.0359 | 0.0094 | 0.1145 | 0.3831 | 0.3339 | 0.0443 | 0.0584 | 0.5375 | 3 |
| rs9617090 | SNV | 0.2737 | 0.0580 | 0.0042 | 0.4381 | 0.2424 | 0.3430 | 0.0020 | 0.1666 | 0.7259 | 0.3036 | 0.0087 | 0.3943 | 3 |
| rs34173813 | SNV | 0.0438 | 0.6542 | 0.1590 | 0.0038 | 0.0100 | 0.0808 | 0.8031 | 0.8130 | 0.5730 | 0.0776 | 0.5269 | 0.9996 | 3 |
| rs28575622 | SNV | 0.2768 | 0.3939 | 0.5037 | 0.0192 | 0.0069 | 0.6824 | 0.1249 | 0.6636 | 0.0497 | 0.8830 | 0.4494 | 0.0598 | 3 |
| rs3811444 | SNV | 0.1907 | 0.5607 | 0.2687 | 0.2185 | 0.1727 | 0.2844 | 0.0659 | 0.9717 | 0.0161 | 0.0766 | 0.0055 | 0.0184 | 3 |
| rs912174 | SNV | 0.0058 | 0.5914 | 0.0013 | 0.0502 | 0.0935 | 0.1459 | 0.1266 | 0.7273 | 0.6289 | 0.0366 | 0.2611 | 0.7548 | 3 |
| rs1200345 | SNV | 0.1024 | 0.8876 | 0.0137 | 0.0658 | 0.2259 | 0.0392 | 0.0034 | 0.6757 | 0.0639 | 0.1037 | 0.1385 | 0.2838 | 3 |
| rs148267944 | SNV | 0.2076 | 0.0307 | 0.5028 | 0.4748 | 0.3369 | 0.4061 | 0.0024 | 0.0083 | 0.0713 | 0.7535 | 0.2170 | 0.0835 | 3 |
| rs10221698 | SNV | 0.5496 | 0.0939 | 0.2732 | 0.7660 | 0.4798 | 0.8611 | 0.0333 | 0.8488 | 0.0003 | 0.8854 | 0.0994 | 0.0194 | 3 |
| rs149478475 | SNV | 0.0616 | 0.1234 | 0.4918 | 0.0123 | 0.0439 | 0.1671 | 0.0010 | 0.7088 | 0.0580 | 0.2310 | 0.1872 | 0.2551 | 3 |
| rs11540014 | SNV | 0.2458 | 0.0908 | 0.7995 | 0.0274 | 0.0562 | 0.3998 | 0.0023 | 0.0607 | 0.0436 | 0.4110 | 0.3792 | 0.6582 | 3 |
| rs144410671 | SNV | 0.0301 | 0.1842 | 0.0775 | 0.0034 | 0.0071 | 0.0780 | 1.0000 | 0.5091 | 0.9124 | 0.0633 | 01131 | 0.1631 | 3 |
| ALP | LAB | 0.0669 | 0.4452 | 0.1031 | 0.5056 | 0.1688 | 0.0364 | 0.0767 | 0.0234 | 0.1840 | 0.0088 | 0.1262 | 0.7418 | 3 |
| rs1250560 | SNV | 0.0155 | 0.9940 | 0.1659 | 0.0132 | 0.0090 | 0.4433 | 0.3820 | 0.9969 | 0.8537 | 0.1123 | 0.3471 | 0.2097 | 3 |
| rs13263453 | SNV | 0.9270 | 0.0012 | 0.6272 | 0.8731 | 0.5631 | 0.2107 | 0.3020 | 0.0397 | 0.3806 | 0.3910 | 0.5657 | 0.0295 | 3 |
| rs10227141 | SNV | 0.0233 | 0.0644 | 0.0527 | 0.2337 | 0.4389 | 0.0026 | 0.2451 | 0.0821 | 0.3224 | 0.0297 | 0.1071 | 0.1390 | 3 |
| rs41273179 | SNV | 0.0025 | 0.4372 | 0.0283 | 0.3196 | 0.1505 | 0.1799 | 0.3448 | 0.4497 | 0.1749 | 0.0337 | 0.0703 | 0.6570 | 3 |
| rs3739435 | SNV | 0.0180 | 0.0903 | 0.0027 | 0.0796 | 0.0565 | 0.8389 | 0.5776 | 0.1839 | 0.5391 | 0.5350 | 0.0282 | 0.2139 | 3 |
| rs1049296 | SNV | 0.0774 | 0.1994 | 0.2377 | 0.2687 | 0.1384 | 0.2632 | 0.0668 | 0.9085 | 0.0026 | 0.0136 | 0.0039 | 0.1009 | 3 |
| chr7:72430225-72430632 | CNV | 0.0384 | 0.4090 | 0.2104 | 0.0065 | 0.0062 | 0.7978 | 0.6240 | 0.2256 | 0.0762 | 0.3471 | 0.0527 | 0.5740 | 3 |
| rs2270915 | SNV | 0.0283 | 0.1117 | 0.1302 | 0.2969 | 0.1892 | 0.0761 | 0.4773 | 0.2361 | 0.0006 | 0.1328 | 0.0152 | 0.4331 | 3 |
| chr21:15013512-15014139 | CNV | 0.0813 | 0.1184 | 0.1679 | 0.3540 | 0.5893 | 0.0025 | 0.0031 | 0.1318 | 0.4374 | 0.0281 | 0.1407 | 0.2432 | 3 |
| rs2271502 | SNV | 0.0240 | 0.3465 | 0.0029 | 0.0443 | 0.1101 | 0.1828 | 0.2268 | 0.4974 | 0.2609 | 0.0811 | 0.1677 | 0.7934 | 3 |
| rs6000181 | SNV | 0 0253 | 04239 | 0 1744 | 0.0057 | 00182 | 0 7283 | 0 3204 | 0.0616 | 0.2240 | 0 5118 | 0 2298 | 0 4782 | 3 |
| rs11258194 | SNV | 0.1203 | 0.0446 | 0.6966 | 0.4039 | 0.5599 | 0.0503 | 0.8984 | 0.7969 | 0.0070 | 0.0506 | 0.0168 | 0.3005 | 3 |
| rs966185 | SNV | 0.1356 | 0.3639 | 0.0239 | 0.0469 | 0.0741 | 0.0817 | 0.0703 | 0.0714 | 0.4676 | 0.0082 | 0.0642 | 0.2340 | 3 |
| rs3924467 | SNV | 0.1297 | 0.0218 | 0.1639 | 0.9755 | 0.5993 | 0.2865 | 0.2830 | 0.8132 | 0.6685 | 0.3093 | 0.0091 | 0.0003 | 3 |
| rs11245936 | SNV | 0.0539 | 0.0080 | 0.8344 | 0.0513 | 0.0117 | 0.2222 | 0.3656 | 0.0063 | 0.1049 | 0.2076 | 0.0631 | 0.5409 | 3 |
| rs879664 | SNV | 0.1196 | 0.4683 | 0.2680 | 0.0762 | 0.0905 | 0.0314 | 0.1269 | 0.6196 | 0.0710 | 0.0058 | 0.0037 | 0.1142 | 3 |
| rs2274271 | SNV | 0.1659 | 0.0214 | 0.2278 | 0.2181 | 0.3816 | 0.1007 | 0.4203 | 0.0083 | 0.3885 | 0.2768 | 0.1159 | 0.0116 | 3 |
| rs17006143 | SNV | 0.0517 | 0.0039 | 0.1351 | 0.0041 | 0.0104 | 0.2810 | 0.1192 | 0.7520 | 0.6412 | 0.5412 | 0.0759 | 0.1751 | 3 |
| rs7761731 | SNV | 0.2851 | 0.5112 | 0.2449 | 0.0028 | 0.0012 | 0.5500 | 0.3660 | 0.9420 | 0.0614 | 0.1724 | 0.0121 | 0.1754 | 3 |
| rs214950 | SNV | 0.0525 | 0.0944 | 0.1077 | 0.1126 | 0.4247 | 0.0130 | 0.2116 | 0.3954 | 0.1238 | 0.0082 | 0.0072 | 0.0515 | 3 |
| chr15:22413530-22413968 | CNV | 0.1862 | 0.0004 | 0.9801 | 0.7245 | 0.4757 | 0.6541 | 0.3537 | 0.0443 | 0.0279 | 0.6371 | 0.1709 | 0.3248 | 3 |
| rs11542470 | SNV | 0.1488 | 0.0926 | 0.7569 | 0.9074 | 0.6630 | 0.2924 | 0.1924 | 0.3156 | 0.0020 | 0.1392 | 0.0454 | 0.0218 | 3 |
| rs146303784 | SNV | 0.1340 | 0.3031 | 0.2147 | 0.8659 | 0.2270 | 0.0135 | 0.1564 | 0.3521 | 0.0123 | 0.0518 | 0.0887 | 0.0406 | 3 |
| rs220111 | SNV | 0.0369 | 0.5270 | 0.7213 | 0.0061 | 0.0057 | 0.1330 | 0.1896 | 0.1628 | 0.4402 | 0.2936 | 0.1522 | 0.9217 | 3 |
| rs220129 | SNV | 0.8421 | 0.1089 | 0.3465 | 0.2328 | 0.4836 | 0.0375 | 0.0228 | 0.0615 | 0.0841 | 0.0517 | 0.3602 | 0.0014 | 3 |
| rs228104 | SNV | 0.0349 | 0.3624 | 0.1811 | 0.0062 | 0.0010 | 0.7959 | 0.5814 | 0.8440 | 0.8724 | 0.3893 | 0.2670 | 0.9234 | 3 |
| chr15:22382207-22382691 | CNV | 0.2341 | 0.0005 | 0.9697 | 0.6789 | 0.5526 | 0.7151 | 0.4473 | 0.0355 | 0.0454 | 0.7485 | 0.2488 | 0.3940 | 3 |
| rs80169011 | SNV | 0.0049 | 0.5099 | 0.2826 | 0.1551 | 0.0117 | 0.1204 | 0.1359 | 0.4893 | 0.1430 | 0.0090 | 0.0551 | 0.6229 | 3 |
| rs11557154 | SNV | 0.1246 | 0.1273 | 0.1724 | 0.4213 | 0.5493 | 0.0050 | 0.0567 | 0.0123 | 0.0157 | 0.0535 | 0.2451 | 0.3425 | 3 |
| rs740223 | SNV | 0.0065 | 0.1988 | 0.0651 | 0.0766 | 0.0039 | 0.1167 | 0.3843 | 0.1661 | 0.7016 | 0.0182 | 0.1755 | 0.6892 | 3 |
| rs150696137 | SNV | 0.1468 | 0.4295 | 0.2715 | 0.0309 | 0.1940 | 0.1126 | 0.5867 | 0.7102 | 0.6290 | 0.0280 | 0.1067 | 0.0057 | 3 |
| rs3762001 | SNV | 0.4124 | 0.0042 | 0.2681 | 0.6940 | 0.7338 | 0.3421 | 0.0746 | 0.0428 | 0.0060 | 0.2825 | 0.6351 | 0.3296 | 3 |
| rs12664213 | SNV | 0.2330 | 0.0867 | 0.1539 | 0.9333 | 0.9828 | 0.0047 | 0.1656 | 0.0593 | 0.0050 | 0.0168 | 0.0921 | 0.0598 | 3 |
| rs34305100 | SNV | 0.0674 | 0.0206 | 0.0766 | 0.7666 | 0.3852 | 0.1946 | 0.1631 | 0.0054 | 0.0027 | 0.2130 | 0.2084 | 0.0955 | 3 |
| rs28739019 | SNV | 0.0058 | 0.1292 | 0 0698 | 0.4015 | 0 0703 | 0 8008 | 0 6368 | 0 0024 | 0 0487 | 0.1105 | 0.0703 | 0.7254 | 3 |
| rs117505788 | SNV | 0.0051 | 0.1161 | 0.0465 | 0.1419 | 0.1772 | 0.3645 | 0.0899 | 0.0014 | 0.2166 | 0.3711 | 0.3218 | 0.2676 | 3 |
| rs9621461 | SNV | 0.2504 | 0.0344 | 0.2341 | 0.2530 | 0.4707 | 0.4634 | 0.0582 | 0.5916 | 0.0139 | 0.1412 | 0.0449 | 0.1961 | 3 |
| rs2311802 | SNV | 0.3961 | 0.0388 | 0.3630 | 0.3367 | 0.8109 | 0.1304 | 0.0041 | 0.1528 | 0.0368 | 0.4535 | 0.0968 | 0.1476 | 3 |
| rs187661008 | SNV | 0.1959 | 0.0813 | 0.2599 | 0.6105 | 0.5654 | 0.5142 | 0.0212 | 0.7815 | 0.0041 | 0.0329 | 0.0992 | 0.0576 | 3 |
| rs181204862 | SNV | 0.0898 | 0.1656 | 0.0304 | 0.6195 | 0.8018 | 0.0444 | 0.1028 | 0.4547 | 0.3009 | 0.0687 | 0.0554 | 0.0010 | 3 |
| rs79193415 | SNV | 0.0184 | 0.0267 | 0.1977 | 0.0111 | 0.0519 | 0.1567 | 0.0575 | 0.0969 | 0.1305 | 0.8327 | 0.3261 | 0.0733 | 3 |
| rs28608091 | SNV | 0.1456 | 0.3662 | 0.3866 | 0.1797 | 0.3132 | 0.0114 | 0.0035 | 0.5835 | 0.0703 | 0.0097 | 0.0734 | 0.4745 | 3 |
| rs74977890 | SNV | 0.1352 | 0.8197 | 0.5786 | 0.0281 | 0.0634 | 0.0084 | 0.2636 | 0.9511 | 0.4415 | 0.0233 | 0.1861 | 0.2731 | 3 |
| rs2274848 | SNV | 0.0088 | 0.3315 | 0.0022 | 0.1295 | 0.0719 | 0.1298 | 0.7375 | 0.3490 | 0.0255 | 0.1971 | 0.1277 | 0.2748 | 3 |
| rs750559 | SNV | 0.0343 | 0.0031 | 0.2359 | 0.0548 | 0.0089 | 0.3841 | 0.8213 | 0.0732 | 0.2686 | 0.5251 | 0.1342 | 0.0933 | 3 |
| rs117992169 | SNV | 0.3401 | 0.0348 | 0.3642 | 0.3113 | 0.2925 | 0.1082 | 0.9132 | 0.9008 | 0.0375 | 0.5416 | 0.1126 | 0.0037 | 3 |
| rs3733414 | SNV | 0.0017 | 0.2392 | 0.0003 | 0.2016 | 0.2736 | 0.1338 | 0.1356 | 0.2548 | 0.1441 | 0.0233 | 0.1274 | 0.7143 | 3 |
| rs2280085 | SNV | 0.0838 | 0.2522 | 0.0928 | 0.0123 | 0.0096 | 0.3223 | 0.3563 | 0.8554 | 0.0252 | 0.1352 | 0.0876 | 0.3035 | 3 |
| rs3804583 | SNV | 0.5005 | 0.0668 | 0.6530 | 0.6243 | 0.4367 | 0.5840 | 0.1357 | 0.0202 | 0.0245 | 0.6000 | 0.2226 | 0.0039 | 3 |
| rs12505221 | SNV | 0.0713 | 0.0045 | 0.2200 | 0.0050 | 0.0020 | 0.5368 | 0.2477 | 0.1665 | 0.2809 | 0.4071 | 0.1380 | 0.0797 | 3 |
| rs3748199 | SNV | 0.1500 | 0.5869 | 0.8243 | 0.0484 | 0.0828 | 0.4515 | 0.6236 | 0.7093 | 0.0043 | 0.0989 | 0.0184 | 0.0854 | 3 |
| rs3813768 | SNV | 0.8431 | 0.1849 | 0.8600 | 0.9294 | 0.4636 | 0.3431 | 0.0023 | 0.3971 | 0.0085 | 0.3028 | 0.3735 | 0.0159 | 3 |
| rs35083184 | SNV | 0.2999 | 0.0749 | 0.2392 | 0.3432 | 0.1633 | 0.1298 | 0.0029 | 0.7263 | 0.0170 | 0.2741 | 0.0218 | 0.0974 | 3 |
| rs3204849 | SNV | 0.2328 | 0.0210 | 0.3106 | 0.1039 | 0.0338 | 0.6444 | 0.5174 | 0.6603 | 0.2521 | 0.9453 | 0.2764 | 0.0010 | 3 |
| rs13963 | SNV | 0.2299 | 0.0252 | 0.6369 | 0.8572 | 0.8127 | 0.9217 | 0.0588 | 0.0019 | 0.5275 | 0.6933 | 0.7929 | 0.0450 | 3 |
| rs2290505 | SNV | 0.3945 | 0.0411 | 0.6664 | 0.0866 | 0.0702 | 0.2877 | 0.1936 | 0.5204 | 0.0022 | 0.3099 | 0.2013 | 0.0070 | 3 |
| rs184908706 | SNV | 0.0851 | 0.0658 | 0.3201 | 0.2126 | 0.2032 | 0.2143 | 0.1275 | 0.0032 | 0.0256 | 0.2115 | 0.0567 | 0.0363 | 3 |
| rs28936679 | SNY | 0.0282 | 0.8271 | 0.0872 | 0.1545 | 0.2586 | 0.0015 | 0.8905 | 0.4035 | 0.2647 | 0.0109 | 0.0673 | 0.8973 | 3 |
| rs1131620 | SNV | 0.0061 | 0.0511 | 0.0195 | 0.3241 | 0.1952 | 0.2843 | 0 0667 | 0 0813 | 0.6930 | 0.0762 | 0.0078 | 0.5586 | 3 |
| rs4682752 | SNV | 0.6851 | 0.0177 | 0.5354 | 0.1536 | 0.1029 | 0.2955 | 0.1713 | 0.0275 | 0.1397 | 0.6711 | 0.1359 | 0.0053 | 3 |
| rs35496730 | SNV | 0.1586 | 0.0103 | 0.8212 | 0.1361 | 0.0866 | 0.6298 | 0.0383 | 0.3688 | 0.0466 | 0.6250 | 0.0853 | 0.0701 | 3 |
| rs8100718 | SNV | 0.1671 | 0.2236 | 0.2795 | 0.7224 | 0.8528 | 0.0013 | 0.0502 | 0.6840 | 0.3054 | 0.0050 | 0.0123 | 0.2306 | 3 |
| rs171140 | SNV | 0 9529 | 0 1175 | 0 1829 | 04851 | 03671 | 0 1868 | 0 0235 | 0 3649 | 0 0299 | 0 5046 | 0 1114 | 0 0034 | 3 |
| rs11228763 | SNV | 0.0272 | 0.1098 | 0.1127 | 0.0116 | 0.1280 | 0.0082 | 0.4284 | 0.1343 | 0.3827 | 0.0585 | 0.0632 | 0.1650 | 3 |
| rs2684348 | SNV | 0.6253 | 0.3584 | 0.2737 | 0.7535 | 0.9129 | 0.5490 | 0.0322 | 0.9946 | 0.0011 | 0.3691 | 0.1043 | 0.0162 | 3 |
| rs73404789 | SNV | 0.1987 | 0.0485 | 0.1231 | 0.0064 | 0.0207 | 0.3686 | 0.8042 | 0.9999 | 0.2944 | 0.7566 | 0.1607 | 0.2800 | 3 |
| rs61746574 | SNV | 0.0282 | 0.2756 | 0.0049 | 0.9897 | 0.6435 | 0.1284 | 0.2306 | 0.4713 | 0.4038 | 0.0832 | 0.0151 | 0.4954 | 3 |
| rs77310684 | SNV | 0.1018 | 0.2259 | 0.4378 | 0.2088 | 0.1221 | 0.0404 | 0.0019 | 0.3405 | 0.8353 | 0.1678 | 0.2053 | 0.0364 | 3 |
| rs57207396 | SNV | 0.0212 | 0.6967 | 0.1744 | 0.2814 | 0.1505 | 0.0077 | 0.7505 | 0.8802 | 0.7326 | 0.0234 | 0.1424 | 0.9985 | 3 |
| chr5:177168045-177168772 | CNV | 0.1671 | 0.2922 | 0.0409 | 0.0033 | 0.0009 | 0.1479 | 0.4616 | 0.4107 | 0.7648 | 0.6153 | 0.0804 | 0.7494 | 3 |
| rs74823804 | SNV | 0.0546 | 0.2065 | 0.1561 | 0.4416 | 0.6336 | 0.2542 | 0.0212 | 0.5783 | 0.0540 | 0.0240 | 0.0164 | 0.3472 | 3 |
| rs4984921 | SNV | 0.6765 | 0.0064 | 0.3034 | 0.5914 | 0.7242 | 0.6158 | 0.0340 | 0.0030 | 0.1958 | 0.3372 | 0.5574 | 0.1039 | 3 |
| rs1860368 | SNV | 0.0013 | 0.7184 | 0.0081 | 0.0961 | 0.0668 | 0.1812 | 0.1925 | 0.5012 | 0.5230 | 0.0644 | 0.0387 | 0.9131 | 3 |
| rs1128864 | SNV | 0.0349 | 0.2729 | 0.1188 | 0.0447 | 0.0101 | 0.3195 | 0.8474 | 0.9666 | 0.3021 | 0.1961 | 0.1068 | 0.8579 | 3 |
| rs607230 | SNV | 0.4734 | 0.0310 | 0.8199 | 0.8295 | 0.8151 | 0.1772 | 0.0951 | 0.0028 | 0.1864 | 0.2951 | 0.6547 | 0.0178 | 3 |
| rs4722064 | SNV | 0.0113 | 0.3616 | 0.0754 | 0.1869 | 0.2154 | 0.2451 | 0 2886 | 0.5689 | 0.1640 | 0.0247 | 0.0066 | 0.2179 | 3 |
| rs9944810 | SNV | 0.0019 | 0.2738 | 0.300€ | 0.0794 | 0.1105 | 0.0686 | 0.5663 | 0.2463 | 0.4382 | 0.0130 | 0.0496 | 0.2671 | 3 |
| rs4857302 | SNV | 0.6786 | 0.1138 | 0.9251 | 0.7138 | 0.6211 | 0.2503 | 0.0417 | 0.5596 | 0.0038 | 0.2910 | 0.2883 | 0.0375 | 3 |
| rs3744550 | SNV | 0.3278 | 0.1650 | 0.4565 | 0.2990 | 0.4878 | 0.0022 | 0.1691 | 0.9110 | 0.0183 | 0.0028 | 0.1199 | 0.0854 | 3 |
| rs56231962 | SNV | 0.0597 | 0.0125 | 0.0742 | 0.3692 | 0.2299 | 0.1168 | 0.0073 | 0.0791 | 0.0748 | 0.1916 | 0.0563 | 0.0375 | 3 |
| rs4805825 | SNV | 0.2744 | 0.3303 | 0.0985 | 0.3171 | 0.6202 | 0.0998 | 0.1757 | 0.2817 | 0.0170 | 0.0407 | 0.0721 | 0.0032 | 3 |
| rs7657817 | SNV | 0.1318 | 0.6734 | 0.0497 | 0.0402 | 0.0102 | 0.9856 | 0.9300 | 0.7340 | 0.3569 | 0.9797 | 0.2286 | 0.6244 | 3 |
| rs78471713 | SNV | 0.0420 | 0.1689 | 0.2560 | 0.0452 | 0.0048 | 0.1774 | 0.0715 | 0.4804 | 0.1337 | 0.3208 | 0.2633 | 0.7822 | 3 |
| rs2241445 | SNV | 0.0085 | 0.2243 | 0.0020 | 0.4702 | 0.2724 | 0.0451 | 0.7658 | 0.2505 | 0.4131 | 0.0653 | 0.2210 | 0.4345 | 3 |
| rs10034345 | SNV | 0.0853 | 0.0156 | 0.0648 | 0.1956 | 0.1331 | 0.1750 | 0.9998 | 0.0060 | 0.2071 | 0.2907 | 0.2141 | 0.0235 | 3 |
| rs9873604 | SNV | 0.4435 | 0.0104 | 0.8915 | 0.1886 | 0.3896 | 0.3592 | 0.2253 | 0.0082 | 0.0714 | 0.5440 | 0.1766 | 0.0014 | 3 |
| rs117039796 | SNV | 0.1143 | 0.0001 | 0.8652 | 0.2742 | 0.7384 | 0.0446 | 0 3126 | 0 0260 | 0.2495 | 0.4205 | 0.0769 | 0.1239 | 3 |
| rs12286721 | SNV | 0.7653 | 0.1639 | 0.4868 | 0.2388 | 0.3020 | 0.0750 | 0.0001 | 0.2097 | 0.1829 | 0.1669 | 0.0233 | 0.0177 | 3 |
| rs2282537 | SNV | 0.2284 | 0.0050 | 0.4143 | 0.0009 | 0.0013 | 0.6413 | 0.1556 | 0.1732 | 0.1253 | 0.5822 | 0.1178 | 0.1135 | 3 |
| rs2276054 | SNV | 0.1560 | 0.3684 | 0.1416 | 0.0350 | 0.0084 | 0.2383 | 0.5222 | 0.4098 | 0.8682 | 0.2124 | 0.3684 | 0.0423 | 3 |
| rs75981854 | SNV | 0.1783 | 0.1243 | 0.2492 | 0.4856 | 0.2173 | 0.0044 | 0.1468 | 0.6344 | 0.1019 | 0.0041 | 0.0039 | 0.0682 | 3 |
| rs2301612 | SNV | 0.0161 | 0.3920 | 0.0879 | 0.0333 | 0.0042 | 0.9441 | 0.7685 | 0.2198 | 0.9670 | 0.5826 | 0.1510 | 0.5266 | 3 |
| rs2291569 | SNV | 0.0587 | 0.0850 | 0.0770 | 0.1502 | 0.1421 | 0.0049 | 0.1077 | 0.0586 | 0.2232 | 0.0041 | 0.0345 | 0.5047 | 3 |
| rs117805838 | SNV | 0.2361 | 0.0228 | 0.2924 | 0.3231 | 0.6476 | 0.0535 | 0.2641 | 0.3068 | 0.0301 | 0.1814 | 0.1018 | 0.0034 | 3 |
| chr5:70307899-70309043 | CNV | 0.0454 | 0.0848 | 0.1841 | 0.6327 | 0.6777 | 0.4246 | 0.3743 | 0.7803 | 0.0502 | 0.3160 | 0.0463 | 0.0058 | 3 |
| rs7249419 | SNV | 0.0944 | 0.1683 | 0.4754 | 0.0764 | 0.0101 | 0.3648 | 0.0083 | 0.1017 | 0.3567 | 0.3493 | 0.0348 | 0.1974 | 3 |
| rs6788319 | SNV | 0.1900 | 0.7122 | 0.0310 | 0.0122 | 0.0068 | 0.1995 | 0.0813 | 0.6167 | 0.3954 | 0.3858 | 0.3239 | 0.6095 | 3 |
| rs818818 | SNV | 0.8243 | 0.0431 | 0.3956 | 0.5733 | 0.4968 | 0.2306 | 0.4086 | 0.2131 | 0.0345 | 0.3683 | 0.0829 | 0.0040 | 3 |
| rs34099167 | SNV | 0.1112 | 0.0700 | 0.6867 | 0.0032 | 0.0032 | 0.1761 | 0.0092 | 0.9008 | 0.5154 | 0.1939 | 0.1075 | 0.0517 | 3 |
| rs2282488 | SNV | 0.0163 | 0.0022 | 0.2982 | 0.0965 | 0.1036 | 0.2800 | 0.2578 | 0.0607 | 0.0923 | 0.1292 | 0.0168 | 0.2393 | 3 |
| rs2291256 | SNV | 0.2607 | 0.6909 | 0.2791 | 0.6235 | 0.8575 | 0.0079 | 0.0094 | 0.9949 | 0.1959 | 0.0307 | 0.1800 | 0.4219 | 3 |
| rs75616428 | SNV | 0.1286 | 0.0448 | 0.4667 | 0.0137 | 0.1039 | 0.2518 | 0.8613 | 0.1354 | 0.1044 | 0.1546 | 0.2081 | 0.0000 | 3 |
| rs1671036 | SNV | 0.6668 | 0.2771 | 0.8812 | 0.5630 | 0.7600 | 0.1610 | 0.0020 | 0.3828 | 0.0846 | 0.0893 | 0.0359 | 0.0185 | 3 |
| rs78536254 | SNV | 0.2314 | 0.8320 | 0.1542 | 0.2018 | 0.1684 | 0.0125 | 0.0015 | 0.4092 | 0.3147 | 0.0230 | 0.0571 | 0.4709 | 3 |
| rs10409531 | SNV | 0.2254 | 0.7889 | 0.0337 | 0.0163 | 0.0073 | 0.2425 | 0.7023 | 0.4229 | 0.6856 | 0.7655 | 0.1800 | 0.9705 | 3 |
| rs3796129 | SNV | 0.2682 | 0.0535 | 0.9369 | 0.0035 | 0.0037 | 0.3564 | 0.8027 | 0.0206 | 0.4686 | 0.7225 | 0.8428 | 0.7154 | 3 |
| rs4789134 | SNV | 0.1583 | 0.0313 | 0.3335 | 0.2305 | 0.3208 | 0.0801 | 0.5208 | 0.0162 | 0.3783 | 0.0706 | 0.0637 | 0.0010 | 3 |
| rs527025 | SNV | 0.2735 | 0.0647 | 0.1936 | 0.0037 | 0.0070 | 0.8460 | 0.4063 | 0.4019 | 0.8374 | 0.8464 | 0.0273 | 0.0879 | 3 |
| rs149088674 | SNV | 0.0801 | 0.0017 | 0.8900 | 0.1596 | 0.3143 | 0.0144 | 0.0604 | 0.0004 | 0.2262 | 0.1611 | 0.0613 | 0.0676 | 3 |
| rs3740861 | SNV | 0.0332 | 0.0783 | 0.1726 | 0.5924 | 0.8766 | 0.1156 | 0.0143 | 0.0895 | 0.0557 | 0.0362 | 0.1285 | 0.7494 | 3 |
| rs34864744 | SNV | 0.1970 | 0.2513 | 0.8084 | 0.0453 | 0.1613 | 0.0146 | 0.2107 | 0.8011 | 0.3052 | 0.0013 | 0.1198 | 0.6601 | 3 |
| rs60732298 | SNV | 0.0859 | 0.1091 | 0.4904 | 0.1030 | 0.1772 | 0.0029 | 0.5075 | 0.4798 | 0.8777 | 0.0198 | 0.2402 | 0.4284 | 2 |
| rs4148304 | SNV | 0.0505 | 0.0957 | 0.9764 | 0.5703 | 0.1256 | 0.0746 | 0.2207 | 0.9317 | 0.0004 | 0.0998 | 0.0165 | 0.2057 | 2 |
| rs80019749 | SNV | 0.0584 | 0.6351 | 0.0333 | 0.0537 | 0.0067 | 0.4238 | 0.7304 | 0.4641 | 0.9614 | 0.8930 | 0.2222 | 0.5866 | 2 |
| rs78774163 | SNV | 0.5528 | 0.3694 | 0.8851 | 0.0106 | 0.0196 | 0.3087 | 0.4502 | 0.9700 | 0.1357 | 0.2950 | 0.6541 | 0.2231 | 2 |
| rs4715 | SNV | 0.0184 | 0.6594 | 0.0026 | 0.6625 | 0.2320 | 0.8142 | 0.7578 | 0.1789 | 0.5462 | 0.1782 | 0.1367 | 0.9048 | 2 |
| rs4963 | SNV | 0.0017 | 0.2262 | 0.0908 | 0.0467 | 0.0850 | 0.1213 | 0.0620 | 0.0855 | 0.4770 | 0.2048 | 0.2580 | 0.4385 | 2 |
| rs3796508 | SNV | 0.1604 | 0.7678 | 0.1919 | 0.5510 | 0.3922 | 0.0081 | 0.8612 | 0.5741 | 0.5905 | 0.0264 | 0.1321 | 0.3620 | 2 |
| rs78872016 | SNV | 0.1078 | 0.6368 | 0.8666 | 0.8514 | 0.8355 | 0.0050 | 0.5343 | 0.0788 | 0.7737 | 0.0069 | 0.3271 | 0.9720 | 2 |
| rs2229642 | SNV | 0.0030 | 0.6853 | 0.0470 | 0.1216 | 0.0568 | 0.3695 | 0.6328 | 0.8362 | 0.8182 | 0.3708 | 0.1299 | 0.9720 | 2 |
| rs79731548 | SNV | 0.1719 | 0.3702 | 0.4762 | 0.0118 | 0.0321 | 0.9084 | 0.1242 | 0.4266 | 0.5481 | 0.9591 | 0.1390 | 0.9357 | 2 |
| rs141504768 | SNV | 0.6052 | 0.0125 | 0.8815 | 0.2751 | 0.3818 | 0.5376 | 0.0690 | 0.3929 | 0.0273 | 0.5533 | 0.1064 | 0.0621 | 2 |
| rs3213869 | SNV | 0.7660 | 0.8002 | 0.7658 | 0.0223 | 0.0098 | 0.7316 | 0.4368 | 0.2829 | 0.5509 | 0.9907 | 0.7380 | 0.5957 | 2 |
| rs10258821 | SNV | 0.1093 | 0.0028 | 0.5509 | 0.3296 | 0.2910 | 0.6237 | 0.6562 | 0.0237 | 0.4535 | 0.6096 | 0.2341 | 0.1790 | 2 |
| rs2280839 | SNV | 0.0296 | 0.1179 | 0.0696 | 0.2199 | 0.3681 | 0.1094 | 0.7690 | 0.0055 | 0.4535 | 0.2609 | 0.1202 | 0.7425 | 2 |
| rs7831467 | SNV | 0.2198 | 0.4189 | 0.5514 | 0.3472 | 0.0933 | 0.0657 | 0.1838 | 0.0442 | 0.6280 | 0.0039 | 0.3394 | 0.4747 | 2 |
| rs17707331 | SNV | 0.0923 | 0.0502 | 0.4347 | 0.0088 | 0.0329 | 0.8311 | 0.8814 | 0.1205 | 0.1496 | 0.1241 | 0.1091 | 0.3979 | 2 |
| rs62490396 | SNV | 0.5666 | 0.0306 | 0.5086 | 0.8932 | 0.7767 | 0.9194 | 0.2270 | 0.0031 | 0.3474 | 0.7778 | 0.1778 | 0.1036 | 2 |
| rs2272095 | SNV | 0.1229 | 0.3508 | 0.3037 | 0.0732 | 0.0936 | 0.3997 | 0.0015 | 0.1350 | 0.6827 | 0.3041 | 0.0438 | 0.1182 | 2 |
| rs74531854 | SNV | 0.5132 | 0.0148 | 0.8253 | 0.9650 | 0.8262 | 0.5140 | 0.2661 | 0.9998 | 0.0391 | 0.3118 | 0.1704 | 0.2058 | 2 |
| rs2233701 | SNV | 0.0757 | 0.4047 | 0.4226 | 0.1895 | 0.3081 | 0.0895 | 0.8219 | 0.8055 | 0.0019 | 0.0520 | 0.0242 | 0.0811 | 2 |
| rs36104932 | SNV | 0.1513 | 0.6127 | 0.0898 | 0.0595 | 0.2139 | 0.0018 | 0.7618 | 0.8903 | 0.8185 | 0.0701 | 0.0378 | 0.9209 | 2 |
| rs72819488 | SNV | 0.0470 | 0.4666 | 0.0971 | 0.6848 | 0.4579 | 0.3041 | 0.0978 | 0.6763 | 0.0714 | 0.0087 | 0.1406 | 0.2108 | 2 |
| rs4077930 | SNV | 0.0539 | 0.0600 | 0.0554 | 0.0369 | 0.0103 | 0.9133 | 0.3345 | 0.2494 | 0.1960 | 0.5676 | 0.0563 | 0.1870 | 2 |
| rs9840317 | SNV | 0.5752 | 0.1048 | 0.7569 | 0.3118 | 0.5275 | 0.2930 | 0.2042 | 0.0232 | 0.0678 | 0.6688 | 0.4075 | 0.0020 | 2 |
| rs2070180 | SNV | 0.6117 | 0.7692 | 0.8011 | 0.5347 | 0.6650 | 0.0058 | 0.6187 | 0.6103 | 0.8423 | 0.0463 | 0.7238 | 0.4235 | 2 |
| rs1044303 | SNV | 0.4956 | 0.2032 | 0.7385 | 0.8956 | 0.6007 | 0.0964 | 0.0034 | 0.7300 | 0.0595 | 0.6357 | 0.1396 | 0.0006 | 2 |
| rs12175588 | SNV | 0.1077 | 0.0521 | 0.8271 | 0.0375 | 0.0840 | 0.1653 | 0.5884 | 0.0078 | 0.6133 | 0.1117 | 0.3886 | 0.3865 | 2 |
| rs114639346 | SNV | 0.1056 | 0.5249 | 0.0144 | 0.2213 | 0.4184 | 0.6183 | 0.0037 | 0.4162 | 0.3867 | 0.3794 | 0.1284 | 0.2593 | 2 |
| rs 17547830 | SNV | 0.0069 | 0.9998 | 0.0494 | 0.1417 | 0.0723 | 0.1351 | 0.9968 | 1.0000 | 0.9996 | 0.0613 | 0.9982 | 0.9972 | 2 |
| rs17661364 | SNV | 0.1702 | 0.0771 | 0.3555 | 0.1819 | 0.1611 | 0.0365 | 0.3150 | 0.3060 | 0.1975 | 0.1116 | 0.0869 | 0.0045 | 2 |
| rs1532268 | SNV | 0.0938 | 0.1804 | 0.9988 | 0.1298 | 0.0082 | 0.5875 | 0.1657 | 0.7425 | 0.0952 | 0.0659 | 0.0259 | 0.9848 | 2 |
| rs76872483 | SNV | 0.0382 | 0.9667 | 0.0047 | 0.3745 | 0.2940 | 0.6116 | 0.3587 | 0.7665 | 0.5703 | 0.5609 | 0.0592 | 0.6194 | 2 |
| rs144481864 | SNV | 0.2255 | 0.0339 | 0.7794 | 0.3741 | 0.1848 | 0.3546 | 1.0000 | 0.0017 | 0.9952 | 0.6788 | 0.8313 | 1.0000 | 2 |
| rs6767237 | SNV | 0.3674 | 0.1267 | 0.7335 | 0.0118 | 0.0175 | 0.3875 | 0.8957 | 0.3851 | 0.3889 | 0.5367 | 0.0982 | 0.3372 | 2 |
| rs10513680 | SNV | 0.0228 | 0.0885 | 0.1726 | 0.6579 | 0.6107 | 0.2784 | 0.5863 | 0.0084 | 0.3485 | 0.1816 | 0.4309 | 0.4589 | 2 |
| rs16875312 | SNV | 0.4118 | 0.1019 | 0.3361 | 0.3549 | 0.3763 | 0.1128 | 0.0271 | 0.1159 | 0.3765 | 0.0890 | 0.0238 | 0.9788 | 2 |
| rs28364680 | SNV | 0.6122 | 0.6013 | 0.7693 | 0.0047 | 0.0392 | 0.1490 | 0.1490 | 0.6802 | 0.3164 | 0.2232 | 0.2237 | 0.1390 | 2 |
| rs3814416 | SNV | 0.1310 | 0.9720 | 0.3023 | 0.0957 | 0.1610 | 0.0005 | 0.9605 | 0.5028 | 0.8609 | 0.0022 | 0.0820 | 0.4821 | 2 |
| rs1972977 | SNV | 0.4662 | 0.3228 | 0.0659 | 0.1794 | 0.3747 | 0.1621 | 0.0039 | 0.4305 | 0.0424 | 0.3092 | 0.0660 | 0.0638 | 2 |
| rs72790006 | SNV | 0.3349 | 0.0150 | 0.9930 | 0.3185 | 0.4359 | 0.5216 | 0.7189 | 0.1240 | 0.8560 | 0.5977 | 0.7859 | 0.0237 | 2 |
| rs1132528 | SNV | 0.1739 | 0.2214 | 0.6329 | 0.4256 | 0.4418 | 0.0977 | 0.3730 | 0.7478 | 0.3660 | 0.0157 | 0.0092 | 0.1922 | 2 |
| rs140120230 | SNV | 0.0156 | 0.1024 | 0.1616 | 0.0609 | 0.1582 | 0.1717 | 0.0019 | 0.2370 | 0.4875 | 0.0508 | 0.4581 | 0.4104 | 2 |
| rs1049402 | SNV | 0.3942 | 0.0763 | 0.2951 | 0.0446 | 0.2956 | 0.1008 | 0.0022 | 0.2130 | 0.1227 | 0.2694 | 0.1154 | 0.1332 | 2 |
| rs11686212 | SNV | 0.8289 | 0.1062 | 0.4305 | 0.1550 | 0.3491 | 0.0241 | 0.4147 | 0.7487 | 0.4467 | 0.1595 | 0.4239 | 0.0044 | 2 |
| rs11547228 | SNV | 0.3446 | 0.2286 | 0.0668 | 0.3038 | 0.3077 | 0.0280 | 0.2015 | 0.7822 | 0.1521 | 0.0956 | 0.0053 | 0.0980 | 2 |
| rs1039084 | SNV | 0.0974 | 0.0669 | 0.9150 | 0.0108 | 0.0049 | 0.5713 | 0.6508 | 0.1684 | 0.1478 | 0.6812 | 0.4511 | 0.3035 | 2 |
| rs1334576 | SNV | 0.0038 | 0.3133 | 0.0012 | 0.1757 | 0.1848 | 0.7135 | 0.1670 | 0.8425 | 0.9476 | 0.4379 | 0.1529 | 0.7353 | 2 |
| rs11130296 | SNV | 0.0987 | 0.9998 | 0.0074 | 0.2532 | 0.7832 | 0.2073 | 0.9122 | 0.8161 | 0.1824 | 0.1503 | 0.0476 | 0.3878 | 2 |
| rs12537531 | SNV | 0.1497 | 0.7423 | 0.2027 | 0.0032 | 0.0148 | 0.6511 | 0.6436 | 0.7485 | 0.4644 | 0.9251 | 0.8933 | 0.5007 | 2 |
| rs38409 | SNV | 0.3657 | 0.0188 | 0.2443 | 0.0866 | 0.3678 | 0.1884 | 0.0022 | 0.0619 | 0.0829 | 0.2517 | 0.0829 | 0.1409 | 2 |
| rs3736001 | SNV | 0.1737 | 0.1658 | 0.3940 | 0.0318 | 0.0082 | 0.7501 | 0.2025 | 0.4124 | 0.3671 | 0.6856 | 0.2066 | 0.1956 | 2 |
| rs74908317 | SNV | 0.4595 | 0.0358 | 0.7246 | 0.2258 | 0.4296 | 0.9289 | 0.4219 | 0.0075 | 0.1462 | 0.7785 | 0.4457 | 0.3047 | 2 |
| rs59901247 | SNV | 0.0158 | 0.5953 | 0.1211 | 0.0734 | 0.0070 | 0.1041 | 0.1331 | 0.6874 | 0.7992 | 0.2215 | 0.6496 | 0.1571 | 2 |
| rs9381994 | SNV | 0.5839 | 0.0417 | 0.0948 | 0.4436 | 0.3948 | 0.0861 | 0.0934 | 0.1142 | 0.0774 | 0.1522 | 0.0665 | 0.0017 | 2 |
| rs9677948 | SNV | 0.0645 | 0.1993 | 0.0562 | 0.0135 | 0.0886 | 0.1663 | 0.3458 | 0.0040 | 0.6495 | 0.3821 | 0.1400 | 0.4248 | 2 |
| rs1226475 | SNV | 0_2022 | 0.4405 | 0.2396 | 0.6864 | 0.8627 | 0.0075 | 0.5690 | 0.9746 | 0.1979 | 0.0025 | 0.0958 | 0.2491 | 2 |
| rs76776064 | SNV | 0.0771 | 0.5589 | 0.0544 | 0.0124 | 0.0067 | 0.3790 | 0.2873 | 0.9930 | 0.7790 | 0.1219 | 0.1203 | 0.9740 | 2 |
| rs2241883 | SNV | 0.0524 | 0.0135 | 0.4529 | 0.2928 | 0.2519 | 0.1724 | 0.1849 | 0.0099 | 0.0856 | 0.2427 | 0.3120 | 0.6004 | 2 |
| rs11575936 | SNV | 0.0626 | 0.6334 | 0.1442 | 0.0104 | 0.0237 | 0.9932 | 0.3400 | 0.8835 | 0.9000 | 0.5768 | 0.1563 | 0.9700 | 2 |
| rs1342642 | SNV | 0.1542 | 0.2487 | 0.2903 | 0.3408 | 0.2286 | 0.3303 | 0.8307 | 0.9127 | 0.0032 | 0.1855 | 0.4904 | 0.0417 | 2 |
| rs937838 | SNV | 0.2804 | 0.0467 | 0.8609 | 0.6566 | 0.8726 | 0.4870 | 0.4584 | 0.0045 | 0.5092 | 0.7937 | 0.9959 | 0.4822 | 2 |
| rs12541381 | SNV | 0.0372 | 0 0837 | 0.1713 | 0.1859 | 0.1976 | 0.3244 | 06712 | 0.0109 | 0.2183 | 0.2861 | 0.2155 | 0 4683 | 2 |
| rs1499280 | SNV | 0.3379 | 0.5932 | 0.5808 | 0.6208 | 0.9016 | 0.6430 | 0.0191 | 0.5089 | 0.1218 | 0.7751 | 0.2245 | 0.0021 | 2 |
| chr10:47416560-47417156 | CNV | 0.0987 | 0.9978 | 0.0543 | 0.0083 | 0.0221 | 0.9991 | 0.9994 | 0.9828 | 0.5242 | 0.9079 | 0.2436 | 0.2988 | 2 |
| rs3731749 | SNV | 0.3016 | 0.4458 | 0.8666 | 0.0457 | 0.0078 | 0.7968 | 0.4945 | 0.3697 | 0.9219 | 0.5515 | 0.6452 | 0.4222 | 2 |
| rs146596728 | SNV | 0.0708 | 0.6704 | 0.0255 | 0.4360 | 0.3833 | 0.1943 | 0.8491 | 0.4760 | 0.0107 | 0.2191 | 0.2122 | 0.4875 | 2 |
| rs2240140 | SNV | 0.2738 | 0.0241 | 0.2485 | 0.8749 | 0.4968 | 0.3151 | 0.0007 | 0.0546 | 0.1305 | 0.5548 | 0.1397 | 0.1544 | 2 |
| rs17684500 | SNV | 0.2230 | 0.0379 | 0.3849 | 0.6613 | 0.5375 | 0.8774 | 0.0857 | 0.0026 | 0.1976 | 0.6848 | 0.3682 | 0.2480 | 2 |
| rs11073922 | SNV | 0.2020 | 0.0097 | 0.2862 | 0.0733 | 0.1808 | 0.0565 | 0.1216 | 0.2599 | 0.0395 | 0.3420 | 0.1156 | 0.0518 | 2 |
| rs28368490 | SNV | 0.7363 | 0.7004 | 0.7202 | 0.8928 | 0.8521 | 0.0083 | 0.4249 | 0.6980 | 0.8679 | 0.0075 | 0.1494 | 0.8927 | 2 |
| rs306197 | SNV | 0.8146 | 0.7589 | 0.1620 | 0.8339 | 0.8205 | 0.5275 | 0.0015 | 0.4579 | 0.2161 | 0.6075 | 0.2529 | 0.0207 | 2 |
| rs61748245 | SNV | 0.2237 | 0.0584 | 0.2384 | 0.2885 | 0.2243 | 0.3878 | 0.6142 | 0.0049 | 0.0648 | 0.1305 | 0.1537 | 0.0045 | 2 |
| rs3759786 | SNV | 0.1148 | 0.0044 | 0.3474 | 0.5452 | 0.3503 | 0.2204 | 0 3691 | 0.1118 | 0 0190 | 0 5960 | 0 1594 | 0.0978 | 2 |
| rs12595158 | SNV | 0.1563 | 0.1082 | 0.3433 | 0.1235 | 0.2402 | 0.1702 | 0.0126 | 0.0106 | 0.6095 | 0.1414 | 0.2379 | 0.3404 | 2 |
| rs1867380 | SNV | 0.2288 | 0.3335 | 0.4613 | 0.8761 | 0.5853 | 0.4832 | 0.5547 | 0.3650 | 0.0035 | 0.5451 | 0.1333 | 0.0140 | 2 |
| rs700519 | SNV | 0.4942 | 0.0463 | 0.5611 | 0.6883 | 0.8501 | 0.2439 | 0.9996 | 0.0018 | 0.2224 | 0.5306 | 0.4991 | 0.9931 | 2 |
| rs116753155 | SNV | 0.1905 | 0.8638 | 0.4172 | 0.0123 | 0.0218 | 0.8560 | 0.1691 | 0.6250 | 0.5222 | 0.9382 | 0.1878 | 0.5081 | 2 |
| rs1197665 | SNV | 0.3567 | 0.1217 | 0.0776 | 0.5625 | 0.3571 | 0.4348 | 0.0005 | 0.0211 | 0.1943 | 0.6884 | 0.1603 | 0.0935 | 2 |
| rs78561092 | SNV | 0.1254 | 0.0612 | 0.1162 | 0.0756 | 0.1108 | 0.8461 | 0.0128 | 0.7076 | 0.0117 | 0.8254 | 0.1029 | 0.7752 | 2 |
| rs4903576 | SNV | 0.0553 | 0.0398 | 0.0071 | 0.8424 | 0.1817 | 0.7387 | 0.4717 | 0.2988 | 0.2821 | 0.6897 | 0.2563 | 0.1735 | 2 |
| rs139619016 | SNV | 0.0793 | 0.0223 | 0.0716 | 0.1444 | 0.2255 | 0.4563 | 0.7926 | 0.6279 | 0.0729 | 0.1611 | 0.1317 | 0.0045 | 2 |
| rs3784039 | SNV | 0.0015 | 0.5192 | 0.0014 | 0.4010 | 0.9453 | 0.2719 | 0.9385 | 0.9033 | 0.7336 | 0.1203 | 0.1671 | 0.3617 | 2 |
| rs35949016 | SNV | 0.1869 | 0.4795 | 0.1368 | 0.4069 | 0.7766 | 0.0250 | 0.1078 | 0.2758 | 0.7838 | 0.0051 | 0.2523 | 0.2737 | 2 |
| rs3742642 | SNV | 0.2829 | 0.3412 | 0.3712 | 0.0038 | 0.0360 | 0.2187 | 0.2277 | 0.6429 | 0.4685 | 0.3229 | 0.1644 | 0.3971 | 2 |
| rs2072616 | SNV | 0.5706 | 0.0525 | 0.5379 | 0.8177 | 0.8294 | 0.7977 | 0.0576 | 0.0027 | 0.1907 | 0.6203 | 0.5844 | 0.0241 | 2 |
| rs71389465 | SNV | 0.0781 | 0.1559 | 0.1391 | 0.1024 | 0.0065 | 0.1350 | 0.7445 | 0.6868 | 0.4496 | 0.2093 | 0.0157 | 0.1400 | 2 |
| rs2271023 | SNV | 0.2254 | 0.0116 | 0.4849 | 0.3544 | 0.5031 | 0.6268 | 0.2835 | 0.0083 | 0.6388 | 0.7874 | 0.3946 | 0.6145 | 2 |
| rs6887 | SNV | 0.0992 | 0.6875 | 0.2348 | 0.0022 | 0.0156 | 0.9974 | 0.8332 | 0.5339 | 0.2065 | 0.3328 | 0.2939 | 0.7747 | 2 |
| rs3743738 | SNV | 0.4278 | 0.0077 | 0.2245 | 0.7325 | 0.6707 | 0.6001 | 0.5512 | 0.0606 | 0.0885 | 0.3710 | 0.1110 | 0.0453 | 2 |
| rs12386117 | SNV | 0.2990 | 0.0412 | 0.5333 | 0.1156 | 0.1691 | 0.5630 | 0.9297 | 0.0045 | 0.5053 | 0.6513 | 0.3611 | 0.4288 | 2 |
| rs76315481 | SNV | 0.1081 | 0.1124 | 0.8406 | 0.1331 | 0.4462 | 0.0411 | 0.4883 | 0.3416 | 0.1884 | 0.0081 | 0.1399 | 0.4530 | 2 |
| rs12961939 | SNV | 0.0770 | 0.7658 | 0.3341 | 0.0109 | 0.0423 | 0.4346 | 0.7398 | 0.8900 | 0.5970 | 0.9628 | 0.7152 | 0.3699 | 2 |
| rs78545242 | SNV | 0.1743 | 0.6019 | 0.2102 | 0.6070 | 0.3205 | 0.0169 | 0.4970 | 0.4059 | 0.7971 | 0.0032 | 0.1012 | 0.9786 | 2 |
| rs11665084 | SNV | 0.3024 | 0.1148 | 0.7665 | 0.3441 | 0.5097 | 0.3100 | 0.0196 | 0.0019 | 0.3334 | 0.2512 | 0.6505 | 0.6703 | 2 |
| rs17187262 | SNV | 0.2201 | 0.0102 | 0.7394 | 0.6539 | 0.3355 | 0.4516 | 0.6367 | 0.0315 | 0.5654 | 0.3714 | 0.3615 | 0.0679 | 2 |
| rs7228940 | SNV | 0.1585 | 0.0697 | 0.3004 | 0.0043 | 0.1383 | 0.6075 | 0.7025 | 0.0070 | 0.4314 | 0.485$ | 0.2186 | 0.0566 | 2 |
| rs61738821 | SNV | 0.2534 | 0.6916 | 0.2909 | 0.0201 | 0.0790 | 0.2404 | 0.0011 | 0.7738 | 0.0587 | 0.1863 | 0.1156 | 0.8845 | 2 |
| rs4071641 | SNV | 0.7836 | 0.2279 | 0.3228 | 0.4667 | 0.2058 | 0.3090 | 0.5223 | 0.0541 | 0.0013 | 0.5561 | 0.0635 | 0.0113 | 2 |
| rs7640 | SNV | 0.0786 | 0.5528 | 0.4147 | 0.3247 | 0.3996 | 0.0077 | 0.2024 | 0.7626 | 0.1695 | 0.0386 | 0.2884 | 0.2585 | 2 |
| rs150073346 | SNV | 0.0125 | 0.5850 | 0.0031 | 0.1006 | 0.1384 | 0.2999 | 0.8220 | 0.4993 | 0.2176 | 0.1437 | 0.0547 | 0.4678 | 2 |
| rs9905604 | SNV | 0.1700 | 0.4610 | 0.7578 | 0.2114 | 0.2121 | 0.0071 | 0.5817 | 0.6613 | 0.7415 | 0.0417 | 0.1769 | 0.4793 | 2 |
| rs12453124 | SNV | 0.3381 | 0.3015 | 0.6037 | 0.0432 | 0.0071 | 0.5713 | 0.6336 | 0.4237 | 0.2362 | 0.8813 | 0.6348 | 0.4761 | 2 |
| rs769427 | SNV | 0.2181 | 0.0351 | 0.3118 | 0.2468 | 0.2025 | 0.2321 | 0.0754 | 0.2596 | 0.2863 | 0.0656 | 0.0059 | 0.2667 | 2 |
| rs12941884 | SNV | 0.0587 | 0.0335 | 0.1557 | 0.0619 | 0.0937 | 0.2265 | 0.8419 | 0.0357 | 0.8496 | 0.1498 | 0.0560 | 0.1699 | 2 |
| rs3760299 | SNV | 0.6192 | 0.1132 | 0.3183 | 0.0857 | 0.1375 | 0.6561 | 0.0017 | 0.9556 | 0.0355 | 0.5002 | 0.2721 | 0.1280 | 2 |
| rs3744558 | SNV | 0.0774 | 0.6462 | 0.0236 | 0.5640 | 0.5147 | 0.0058 | 0.2061 | 0.5525 | 0.2456 | 0.2013 | 0.2465 | 0.8322 | 2 |
| rs2078478 | SNV | 0.0013 | 0.4630 | 0.0926 | 0.1093 | 0.0406 | 0.2347 | 0.9832 | 0.4810 | 0.5578 | 0.0980 | 0.1421 | 0.9200 | 2 |
| rs1005190 | SNV | 0.6206 | 0.1721 | 0.3772 | 0.1792 | 0.1545 | 0.2052 | 0.1382 | 0.8117 | 0.0318 | 0.0856 | 0.1426 | 0.0043 | 2 |
| rs10483261 | SNV | 0.8940 | 0.0192 | 0.1631 | 0.8506 | 0.7689 | 0.5486 | 0.5014 | 0.0032 | 0.9076 | 0.3969 | 0.5076 | 0.1247 | 2 |
| rs3827906 | SNV | 0.1363 | 0.0578 | 0.6300 | 0.8627 | 0.2304 | 0.1887 | 0.3192 | 0.0095 | 0.7840 | 0.0268 | 0.3519 | 0.1598 | 2 |
| rs2792146 | SNV | 0.0015 | 0.2881 | 0.1183 | 0.3202 | 0.1072 | 0.3324 | 0.3836 | 0.8972 | 0.2013 | 0.1007 | 0.0284 | 0.4119 | 2 |
| rs2236493 | SNV | 0.3420 | 0.6851 | 0.5470 | 0.2924 | 0.4856 | 0.0035 | 0.1814 | 0.8509 | 0.0985 | 0.0096 | 0.5787 | 0.9334 | 2 |
| rs17847388 | SNV | 0.3208 | 0.0353 | 0.5500 | 0.7734 | 0.6824 | 0.4681 | 0.4076 | 0.0057 | 0.9801 | 0.7619 | 0.8667 | 0.1503 | 2 |
| HLA-DRB1*09:01 | HLA | 0.0609 | 0.3411 | 0.3142 | 0.0032 | 0.0064 | 0.4246 | 0.2690 | 0.2461 | 0.3230 | 0.2283 | 0.0555 | 0.6557 | 2 |
| HLA-B*46:01 | HLA | 0.1395 | 0.0860 | 0.1795 | 0.2305 | 0.3671 | 0.1286 | 0.1932 | 0.0535 | 0.0051 | 0.3993 | 0.0491 | 0.2926 | 2 |
| HLA-B*38:02 | HLA | 0.0784 | 0.0408 | 0.1209 | 0.9598 | 0.7973 | 0.4473 | 0.5332 | 0.0025 | 0.2614 | 0.3786 | 0.2182 | 0.0786 | 2 |
| chr8:7320000-7320652 | CNV | 0 5823 | 0.1919 | 0.4331 | 0.1997 | 0.1697 | 0.0124 | 0.7716 | 0.0063 | 0.9597 | 0.0814 | 0 3395 | 0.6448 | 2 |
| chr8:54793570-54793940 | CNV | 0.0034 | 0.9992 | 0.0699 | 0.9996 | 0.0529 | 0.0903 | 0.9996 | 0.9998 | 0.2340 | 0.0816 | 0.0365 | 0.9995 | 2 |
| chr7:6825302-6825643 | CNV | 0.0186 | 0.4663 | 0.0772 | 0.2081 | 0.3035 | 0.2407 | 0.0029 | 0.2945 | 0.1705 | 0.0988 | 0.0953 | 0.6656 | 2 |
| chr6:29910896-29911491 | CNV | 0.3021 | 0.3840 | 0.2177 | 0.6825 | 0.4692 | 0.3838 | 0.0014 | 0.1858 | 0.0156 | 0.3362 | 0.1084 | 0.1023 | 2 |
| chr6:29910586-29910895 | CNV | 0.2283 | 0.3855 | 0.1955 | 0.3247 | 0.1953 | 0.1947 | 0.0015 | 0.1837 | 0.0172 | 0.2028 | 0.1368 | 0.0737 | 2 |
| chr5:96248068-96248741 | CNV | 0 1388 | 0.4086 | 0.0729 | 0.1993 | 0.1533 | 0.1880 | 0 9998 | 0.1873 | 0.0096 | 0 1377 | 0 0374 | 0.9998 | 2 |
| chr4:69341748-69342476 | CNV | 0.0135 | 0.3199 | 0.0092 | 0.1437 | 0.0713 | 0.0934 | 0.1522 | 0.7004 | 0.1806 | 0.2894 | 0.2699 | 0.3372 | 2 |
| chr3:195390807-195391406 | CNV | 0.0422 | 0.4532 | 0.3207 | 0.7203 | 0.2272 | 0.0790 | 0.1177 | 0.5595 | 0.2980 | 0.0097 | 0.0815 | 0.9515 | 2 |
| chr2:98161877-98162528 | CNV | 0.0962 | 0.8580 | 0.4022 | 0.7464 | 0.9380 | 0.0187 | 0.0868 | 0.2048 | 0.3088 | 0.0096 | 0.0925 | 0.5760 | 2 |
| chr1:40240339-40240976 | CNV | 0.0371 | 0.6921 | 0.0027 | 0.1036 | 0.3041 | 0.7844 | 0.4968 | 0.2973 | 0.4783 | 0.2682 | 0.7533 | 0.2853 | 2 |
| chr1:17087460-17087845 | CNV | 0.1000 | 0.1965 | 0.2272 | 0.0734 | 0.0192 | 0.1006 | 0.3549 | 0.6830 | 0.0085 | 0.2177 | 0.1296 | 0.8910 | 2 |
| chr17:77812551-77812932 | CNV | 0.0071 | 0.1087 | 0.0815 | 0.1112 | 0.0671 | 0.2267 | 0.1862 | 0.2240 | 0.1094 | 0.1051 | 0.0457 | 0.1886 | 2 |
| chr16:55853240-55853860 | CNV | 0.0587 | 0.0778 | 0.7258 | 0.0063 | 0.0106 | 0.2252 | 0.7429 | 0.269: | 0.2485 | 0.1942 | 0.0535 | 0.1908 | 2 |
| chr15:22368319-22368667 | CNV | 0.2760 | 0.0007 | 0.9987 | 0.6506 | 0.5492 | 0.8751 | 0.5804 | 0.0356 | 0.0771 | 0.9320 | 0.3152 | 0.5004 | 2 |
| rs2230469 | SNV | 0.1720 | 0.7982 | 0.1389 | 0.8835 | 0.7611 | 0.0015 | 0.0847 | 0.3127 | 0.2974 | 0.0055 | 0.2408 | 0.8786 | 2 |
| rs2252996 | SNV | 0.3460 | 0.0168 | 0.2831 | 0.0638 | 0.2839 | 0.1848 | 0.1429 | 0.0025 | 0.2132 | 0.1865 | 0.2195 | 0.0680 | 2 |
| rs591157 | SNV | 0.2020 | 0.6681 | 0.4533 | 0.5576 | 0.4972 | 0.0047 | 0.2306 | 0.8240 | 0.6130 | 0.0321 | 0.1452 | 0.7024 | 2 |
| rs59261129 | SNV | 0.0820 | 0.2898 | 0.1179 | 0.0079 | 0.0171 | 0.2133 | 0.1484 | 0.8096 | 0.9264 | 0.6849 | 0.1321 | 0.3844 | 2 |
| rs144408326 | SNV | 0.1996 | 0.1437 | 0.2320 | 0.0035 | 0.0233 | 0.8374 | 0.3067 | 0.2024 | 0.6746 | 0.7819 | 0.6784 | 0.1069 | 2 |
| rs3764147 | SNV | 0.1384 | 0.5573 | 0.0280 | 0.5540 | 0.7427 | 0.3491 | 0.0011 | 0.9744 | 0.3027 | 0.1788 | 0.1112 | 0.1257 | 2 |
| rs144806444 | SNV | 0.1778 | 0.1060 | 0.7950 | 0.2964 | 0.2969 | 0.1717 | 0.0073 | 0.0408 | 0.7088 | 0.2161 | 0.3410 | 0.1149 | 2 |
| rs1056820 | SNV | 0.2413 | 0.3192 | 0.2267 | 0.0038 | 0.0188 | 0.3931 | 0.8851 | 0.6999 | 0.7698 | 0.6627 | 0.6227 | 0.2620 | 2 |
| rs150932292 | SNV | 0.4546 | 0.5063 | 0.3253 | 0.8271 | 0.9559 | 0.0071 | 0.5280 | | 0.9233 | 0.0441 | 0.2446 | 0.2355 | 2 |
| rs41308564 | SNV | 0.2078 | 0.2910 | 0.9097 | 0.1292 | 0.1475 | 0.0634 | 0.7994 | 0.0145 | 0.0708 | 0.0461 | 0.3372 | 0.8368 | 2 |
| rs7312075 | SNV | 0.1577 | 0.3222 | 0.3979 | 0.7750 | 0.8542 | 0.0320 | 0.2929 | 0.8391 | 0.1034 | 0.0029 | 0.1923 | 0.0956 | 2 |
| rs116901253 | SNV | 0.0820 | 0.2898 | 0.1179 | 0.0079 | 0.0171 | 0.2133 | 0.1484 | 0.8096 | 0.9264 | 0.6849 | 0.1321 | 0.3844 | 2 |
| rs17821405 | SNV | 0.0710 | 0.0022 | 0.2838 | 0.4809 | 0.0992 | 0.2570 | 0.3582 | 0.0038 | 0.3445 | 0.2075 | 0.2664 | 0.0948 | 2 |
| rs75116260 | SNV | 0.1015 | 0.0063 | 0.2628 | | 0.8133 | 0.0921 | 0.4587 | 0.0817 | 0.0362 | 0.1265 | 0.2492 | 0.3395 | 2 |
| rs1169288 | SNV | 0.6133 | 0.6824 | 0.0032 | 0.7579 | 0.5836 | 0.7875 | 0.2144 | 0.4835 | 0.3831 | 0.4650 | 0.0440 | 0.6713 | 2 |
| rs7966100 | SNV | 0.2706 | 0.5478 | 0.4509 | 0.0034 | 0.0257 | 0.6300 | 0.5591 | 0.6377 | 0.6758 | 0.3901 | 0.3572 | 0.1190 | 2 |
| rs11020845 | SNV | 0.0541 | 0.2775 | 0.3448 | 0.2714 | 0.0794 | 0.0282 | 0.5808 | 0.4033 | 0.3510 | 0.0075 | 0.2989 | 0.3119 | 2 |
| rs74359608 | SNV | 0.2251 | 0.7586 | 0.5770 | 0.0422 | 0.0083 | 0.7793 | 0.4686 | 0.7478 | 0.2225 | 0.8883 | 0.9539 | 0.9387 | 2 |
| rs239259 | SNV | 0.6164 | 0.5162 | 0.9719 | 0.3485 | 0.4325 | 0.6772 | 0.0094 | 0.9781 | 0.0043 | 0.3372 | 0.2680 | 0.3869 | 2 |
| rs3741252 | SNV | 0.0691 | 0.4946 | 0.4950 | 0.0036 | 0.0047 | 0.8783 | 0.3412 | 0.9262 | 0.8902 | 0.8249 | 0.4446 | 0.8514 | 2 |
| rs998544 | SNV | 0.7678 | 0.8988 | 0.3469 | 0.9649 | 0.8233 | 0.0051 | 0.0006 | 0.6138 | 0.3469 | 0.1408 | 0.3734 | 0.1763 | 2 |
| rs10896271 | SNV | 0.6954 | 0.8783 | 0.4402 | 0.9649 | 0.8233 | 0.0096 | 0.0006 | 0.6138 | 0.5043 | 0.1956 | 0.4699 | 0.3262 | 2 |
| rs4798791 | SNV | 0.8258 | 0.0397 | 0.3552 | 0.6990 | 0.7269 | 0.2522 | 0.8355 | 0.0004 | 0.3766 | 0.3162 | 0.9360 | 0.5479 | 2 |
| rs60854092 | SNV | 0.0879 | 0.1380 | 0.0800 | 0.0290 | 0.0046 | 0.2299 | 0.9090 | 0.3629 | 0.7999 | 0.3160 | 0.6854 | 0.3257 | 2 |
| rs1205434 | SNV | 0.3219 | 0.0035 | 0.6225 | 0.6420 | 0.4695 | 0.0537 | 0.2065 | 0.0856 | 0.0496 | 0.3165 | 0.2039 | 0.0579 | 2 |
| rs118089321 | SNV | 0.0631 | 0.1950 | 0.2411 | 0.0063 | 0.0258 | 0.9998 | 0.2400 | 0.1468 | 0.5171 | 0.6070 | 0.6623 | 0.9997 | 2 |
| rs16983412 | SNV | 0.2656 | 0.1894 | 0.3179 | 0.7878 | 0.6635 | 0.1542 | 0.5540 | 0.7525 | 0.0489 | 0.2095 | 0.1425 | 0.0008 | 2 |
| rs4823054 | SNV | 0.1852 | 0.0075 | 0.9594 | 0.4502 | 0.1114 | 0.5997 | 0.8621 | 0.0068 | 0.3494 | 0.3430 | 0.4988 | 0.8628 | 2 |
| rs117301131 | SNV | 0.0866 | 0.0124 | 0.8805 | 0.1220 | 0.1947 | 0.1144 | 0.1881 | 0.0064 | 0.1194 | 0.0769 | 0.3418 | 0.0540 | 2 |
| rs925878 | SNV | 0.0648 | 0.4491 | 0.3495 | 0.0916 | 0.0820 | 0.0340 | 0.4494 | 0.6442 | 0.3068 | 0.0062 | 0.1123 | 0.7043 | 2 |
| rs361566 | SNV | 0.1559 | 0.2740 | 0.7490 | 0.0052 | 0.0128 | 0.2994 | 0.8350 | 0.0884 | 0.3055 | 0.2278 | 0.2082 | 0.1337 | 2 |
| rs113449283 | SNV | 0.0101 | 0.3299 | 0.0034 | 0.1880 | 0.0560 | 0.1562 | 0.7095 | 0.2621 | 0.8738 | 0.1670 | 0.2996 | 0.1769 | 2 |
| rs2277998 | SNV | 0.0812 | 0.1201 | 0.1639 | 0.0031 | 0.0002 | 0.5376 | 0.5243 | 0.2911 | 0.6691 | 0.4190 | 0.0940 | 0.4564 | 2 |
| rs3810510 | SNV | 0.0722 | 0.5828 | 0.2033 | 0.1071 | 0.6758 | 0.0368 | 0.9124 | 0.6001 | 0.4661 | 0.0063 | 0.7499 | 0.3756 | 2 |
| rs34335016 | SNV | 0.5942 | 0.0229 | 0.6030 | 0.8079 | 0.6071 | 0.1996 | 0.1837 | 0.0015 | 0.0635 | 0.4344 | 0.0624 | 0.0919 | 2 |
| rs863362 | SNV | 0.0831 | 0.9133 | 0.5487 | 0.0113 | 0.0095 | 0.6989 | 0.4054 | 0.9453 | 0.4173 | 0.4140 | 0.2878 | 0.7539 | 2 |
| rs3738648 | SNV | 0.1855 | 0.8112 | 0.1346 | 0.0028 | 0.0229 | 0.1354 | 0.2094 | 0.4288 | 0.2244 | 0.2485 | 0.0973 | 0.1659 | 2 |
| rs76528638 | SNV | 0.0829 | 0.0747 | 0.1144 | 0.0750 | 0.0364 | 0.3857 | 0.8263 | 0.9644 | 0.0928 | 0.5560 | 0.1432 | 0.0019 | 2 |
| rs3750300 | SNV | 0.3624 | 0.0472 | 0.3723 | 0.3355 | 0.1292 | 0.4044 | 0.1885 | 0.0169 | 0.3733 | 0.2459 | 0.1983 | 0.2193 | 2 |
| rs4915221 | SNV | 0.1986 | 0.3042 | 0.3435 | 0.7216 | 0.7444 | 0.2094 | 0.0036 | 0.7307 | 0.1102 | 0.6478 | 0.4251 | 0.0380 | 2 |
| rs788795 | SNV | 0.7771 | 0.0619 | 0.2344 | 0.2684 | 0.2268 | 0.2449 | 0.0010 | 0.1369 | 0.0790 | 0.5037 | 0.0236 | 0.0794 | 2 |
| rs11699009 | SNV | 0.1014 | 0 5727 | 0.3707 | 0.0053 | 0.0106 | 0 2405 | 0.5572 | 0.4148 | 0.4251 | 0 1976 | 0.3396 | 0.1370 | 2 |
| rs2424943 | SNV | 0.0966 | 0.3826 | 0.2939 | 0.0108 | 0.0050 | 0.0770 | 0.1690 | 0.1880 | 0.5004 | 0.2142 | 0.1293 | 0.4256 | 2 |
| rs11118075 | SNV | 0.0440 | 0.3842 | 0.1202 | 0.0824 | 0.0005 | 0.5650 | 0.2567 | 0.7522 | 0.3943 | 0.2596 | 0.3279 | 0.6716 | 2 |
| rs12141189 | SNV | 0.1394 | 0.8107 | 0.4538 | 0.0333 | 0.0084 | 0.3620 | 0.6475 | 0.2875 | 0.1280 | 0.5737 | 0.9590 | 0.4725 | 2 |
| rs3738046 | SNV | 0.0778 | 0.1112 | 0.3815 | 0.2141 | 0.0988 | 0.3815 | 0.0467 | 0.0017 | 0.3151 | 0.2917 | 0.2652 | 0.7360 | 2 |
| rs55760713 | SNV | 0.1490 | 0.0859 | 0.0608 | 0.0904 | 0.1667 | 0.1409 | 0.9998 | 0.9994 | 0.0120 | 0.0487 | 0.0872 | 0.1826 | 2 |
| rs3795375 | SNV | 0.2323 | 0.0020 | 0.5449 | 0.5314 | 0.5923 | 0.3170 | 0.0353 | 0.0834 | 0.1261 | 0.3459 | 0.6885 | 0.4831 | 2 |
| rs34961909 | SNV | 0.2462 | 0.0022 | 0.9565 | 0.1377 | 0.0616 | 0.3762 | 0.3497 | 0.1865 | 0.3521 | 0.1478 | 0.2971 | 0.0315 | 2 |
| rs76258939 | SNV | 0.1769 | 0.0138 | 0.8911 | 0.0980 | 0.2101 | 0.0996 | 0.1276 | 0.0146 | 0.0677 | 0.1590 | 0.1226 | 0.1818 | 2 |
| rs117287351 | SNV | 0.3351 | 0.9003 | 0.9053 | 0.0097 | 0.0211 | 0.9278 | 0.9122 | 0.9968 | 0.6508 | 0.6794 | 0.4524 | 0.9757 | 2 |
| rs118018567 | SNV | 0.1343 | 0.3639 | 0.1618 | 1.0000 | 0.4738 | 0.0808 | 0.9998 | 0.9994 | 0.3206 | 0.0419 | 0.0895 | 0.0060 | 2 |
| rs1156281 | SNV | 0.2430 | 0.6489 | 0.9803 | 0.3450 | 0.6290 | 0.0237 | 0.2195 | 0.3805 | 0.3261 | 0.0063 | 0.5583 | 0.7065 | 2 |
| rs80333860 | SNV | 0.1588 | 0.9596 | 0.3378 | 0.0119 | 0.0634 | 0.5447 | 0.5142 | 0.5977 | 0.0355 | 0.4241 | 0.0929 | 0.3307 | 2 |
| rs8110393 | SNV | 0.9830 | 0.0876 | 0.5178 | 0.4380 | 0.6120 | 0.0551 | 0.1193 | 0.0424 | 0.2133 | 0.6137 | 0.1948 | 0.0038 | 2 |
| rs73888468 | SNV | 0.0065 | 0.0736 | 0.1025 | 0.0603 | 0.0281 | 0.0636 | 0.9981 | 0.2876 | 0.9581 | 0.0613 | 0.0601 | 0.2267 | 2 |
| rs1126458 | SNV | 0.2822 | 0.0071 | 0.8942 | 0.4343 | 0.4283 | 0.5886 | 0.5604 | 0.0430 | 0.4759 | 0.9961 | 0.3286 | 0.2079 | 2 |
| rs28382942 | SNV | 0.0502 | 0.5462 | 0.6020 | 0.0466 | 0.0123 | 0.9959 | 0.5272 | 0.7202 | 0.8291 | 0.0916 | 0.2275 | 0.9992 | 2 |
| rs1644634 | SNV | 0.1770 | 0.7051 | 0.2974 | 0.0261 | 0.0022 | 0.7045 | 0.4173 | 0.4690 | 0.7300 | 0.6311 | 0.2778 | 0.1479 | 2 |
| rs45532635 | SNV | 0.2180 | 0.5344 | 0.7366 | 0.0033 | 0.0473 | 0.7061 | 0.8630 | 0.5543 | 0.7600 | 0.9233 | 0.5454 | 0.8036 | 2 |
| rs79478 | SNV | 0.0009 | 0.3413 | 0.0126 | 0.1657 | 0.0692 | 0.4266 | 0.2944 | 0.8081 | 0.8867 | 0.0785 | 0.2009 | 0.9071 | 2 |
| rs7285167 | SNV | 0.0886 | 0.9947 | 0.1312 | 0.0014 | 0.0028 | 0.9593 | 0.3338 | 0.2025 | 0.8831 | 0.4116 | 0.1685 | 0.2585 | 2 |
| rs1688016 | SNV | 0.1129 | 0.1010 | 0.6501 | 0.8563 | 0.3925 | 0.0208 | 0.0510 | 0.4362 | 0.7152 | 0 0068 | 0.1161 | 0.1729 | 2 |
| rs34050666 | SNV | 0.9989 | 0.8513 | 0.2972 | 0.4523 | 0.4764 | 0.7626 | 0.0323 | 0.9445 | 0.6602 | 0.4584 | 0.5647 | 0.4068 | 1 |
| rs2276360 | SNV | 0.9490 | 0.4820 | 0.8194 | 0.7301 | 0.7877 | 0.2960 | 0.2046 | 0.7319 | 0.8793 | 0.4449 | 0.6604 | 0.0034 | 1 |
| rs948060 | SNV | 0.0034 | 0.7062 | 0.299 | 0.4139 | 0.2852 | 0.6710 | 0.4609 | 0.7554 | 0.8308 | 0.3730 | 0.4219 | 0.4244 | 1 |
| rs6960270 | SNV | 0.0023 | 0.2909 | 0.2369 | 0.1413 | 0.0680 | 0.2524 | 0.9949 | 0.7804 | 0.5684 | 0.1684 | 0.1642 | 0.9033 | 1 |
| rs12420076 | SNV | 0.0008 | 0.2153 | 0.1903 | 0.1849 | 0.1386 | 0.1699 | 0.2777 | 0.8614 | 0.4569 | 0.0853 | 0.1484 | 0.9198 | 1 |
| rs10896516 | SNU | 0.0008 | 0.2153 | 0.1903 | 0.1849 | 0.1386 | 0.1699 | 0.2777 | 0.8614 | 0.4569 | 0.0853 | 0.1484 | 0.9198 | 1 |
| rs2228976 | SNV | 0.1327 | 0.0707 | 0.2591 | 0.8352 | 0.3296 | 0.1790 | 0.3517 | 0.0523 | 0.0008 | 0.1339 | 0.0555 | 0.1311 | 1 |
| rs112731808 | SNV | 0.4943 | 0.3317 | 0.6679 | 0.7368 | 0.5547 | 0.3219 | 0.8192 | 0.0004 | 0.9339 | 0.3646 | 0.6396 | 0.3119 | 1 |
| rs3816182 | SNV | 0.7470 | 0.5515 | 0.2692 | 0.4431 | 0.4336 | 0.8455 | 0.8475 | 0.5513 | 0.9997 | 0.7831 | 0.7280 | 0.0050 | 1 |
| chr19:44932636-44933058 | CNV | 0.3157 | 0.3035 | 0.5208 | 0.4652 | 0.4728 | 0.1534 | 0.0022 | 0 4711 | 0.0683 | 0 0641 | 0.1605 | 0.4521 | 1 |
| rs10896659 | SNV | 0.4275 | 0.0620 | 0.0631 | 0.6021 | 0.2803 | 0.0836 | 0.1712 | 0.7249 | 0.3647 | 0.1342 | 0.1682 | 0.0037 | 1 |
| rs4598671 | SNV | 0.9544 | 0.0970 | 0.2051 | 0.5950 | 0.3318 | 0.1887 | 0.3952 | 0.8347 | 0.4027 | 0.3859 | 0.3696 | 0.0025 | 1 |
| rs3735386 | SNV | 0.6355 | 0.0129 | 0.4917 | 0.3109 | 0.5258 | 0.7095 | 0.2055 | 0.1202 | 0.7072 | 0.5849 | 0.6776 | 0.8667 | 1 |
| rs3808782 | SNV | 0.9087 | 0.2086 | 0.7981 | 0.7394 | 0.8731 | 0.3072 | 0.6596 | 0.8079 | 0.0038 | 0.7872 | 0.5160 | 0.3433 | 1 |
| rs2259633 | SNV | 0.0897 | 0.6506 | 0.9812 | 0.9112 | 0.9983 | 0.7273 | 0.0821 | 0.1695 | 0.1326 | 0.8832 | 0.5941 | 0.0052 | 1 |
| rs10197514 | SNV | 0.7157 | 0.2098 | 0.2686 | 0.4509 | 0.6474 | 0.9879 | 0.2261 | 0.5767 | 0.0030 | 0.5127 | 0.0760 | 0.0646 | 1 |
| rs7975161 | SNV | 0.1457 | 0.2495 | 0.9006 | 0.8760 | 0.5253 | 0.5751 | 0.2106 | 0.1249 | 0.0772 | 0.9870 | 0.4181 | 0.0028 | 1 |
| rs10783231 | SNV | 0.3606 | 0.6733 | 0.3976 | 0.7163 | 0.8313 | 0.4699 | 0.3805 | 0.0147 | 0.6227 | 0.5537 | 0.7813 | 0.6886 | 1 |
| rs2272051 | SNV | 0.5639 | 0.3060 | 0.4607 | 0.7358 | 0.4427 | 0.2540 | 0.3975 | 0.4585 | 0.0572 | 0.8341 | 0.3347 | 0.0052 | 1 |
| rs148844278 | SNV | 0.1340 | 0.0893 | 0.5345 | 0.2672 | 0.1433 | 0.4893 | 1.0000 | 0.0020 | 1.0000 | 0.9494 | 0.9152 | 0.4205 | 1 |
| rs144430026 | SNV | 0.2129 | 0.0112 | 0.6567 | 0.3324 | 0.1276 | 0.9619 | 0.8763 | 0.1011 | 0.1690 | 0.7240 | 0.1925 | 0.3220 | 1 |
| rs4147865 | SNV | 0.1504 | 0.0803 | 0.1863 | 0.5660 | 0.3478 | 0.6723 | 0.0641 | 0.0025 | 0.2730 | 0.5139 | 0.1874 | 0.5523 | 1 |
| rs117231086 | SNV | 0.2649 | 0.4467 | 0.5631 | 0.1917 | 0.2149 | 0.6442 | 0.7925 | 0.0414 | 0.4442 | 0.5280 | 0.9765 | 0.8582 | 1 |
| rs12001918 | SNV | 0.4839 | 0.5634 | 0.5715 | 0.0094 | 0.0874 | 0.9924 | 0.4495 | 0.1363 | 0.7774 | 0.5809 | 0.6230 | 0.9888 | 1 |
| chr14:45599029-45599398 | CNV | 0.4119 | 0.6771 | 0.4679 | 0.8102 | 0.9799 | 0.7400 | 0.0045 | 0.9860 | 0.5675 | 0.2107 | 0.1069 | 0.2945 | 1 |
| rs6968084 | SNV | 0.5642 | 0.0039 | 0.4512 | 0.2958 | 0.3652 | 0.7814 | 0.2296 | 0.0826 | 0.9551 | 0.8120 | 0.3977 | 0.5813 | 1 |
| rs10502 | SNV | 0.7959 | 0.7284 | 0.4345 | 0.2474 | 0.3175 | 0.3654 | 0.1346 | 0.7941 | 0.5499 | 0.4567 | 0.0808 | 0.0029 | 1 |
| rs149645105 | SNV | 0.0046 | 0.2959 | 0.1240 | 0.4270 | 0.1851 | 0.2442 | 0.9997 | 0.9932 | 0.1866 | 0.1591 | 0.1277 | 0.8313 | 1 |
| chr14:74003851-74004198 | CNV | 0.3008 | 0.2446 | 0.0790 | 0.9480 | 0.3961 | 0.6813 | 0.2996 | 0.1015 | 0.3777 | 0.3132 | 0.1780 | 0.0060 | 1 |
| rs11020968 | SNV | 0.1163 | 0.9544 | 0.0026 | 0.4710 | 0.4071 | 0.4151 | 0.2129 | 0.4076 | 0.6997 | 0.3964 | 0.0632 | 0.2698 | 1 |
| rs77442027 | SNV | 0.1917 | 0.4545 | 0.2044 | 0.0062 | 0.0554 | 0.5529 | 0.5774 | 0.8630 | 0.1521 | 0.9103 | 0.2932 | 0.2914 | 1 |
| rs35546150 | SNV | 0.2973 | 0.1115 | 0.4376 | 0.5781 | 0.6622 | 0.6027 | 0.1013 | 0.7924 | 0.0085 | 0.4618 | 0.2366 | 0.1056 | 1 |
| rs17035364 | SNV | 0.3076 | 0.2382 | 0.8720 | 0.5690 | 0.3760 | 0.5042 | 0.2775 | 1.0000 | 0.0058 | 0.3381 | 0.2414 | 0.0783 | 1 |
| chr19:52149405-52149816 | CNV | 0.4639 | 0.0730 | 0.8165 | 0.9367 | 0.8299 | 0.4370 | 0.3383 | 0.6420 | 0.0078 | 0.9966 | 0.4437 | 0.4989 | 1 |
| rs9282734 | SNV | 0.2170 | 0.2306 | 0.6222 | 0.8393 | 0.7721 | 0.0732 | 0.0307 | 0.5403 | 0.7384 | 0.1381 | 0.4628 | 0.2149 | 1 |
| chr1:35370310-35370516 | CNV | 0.0070 | 1.0000 | 0.0660 | | 0.9988 | 0.1870 | 0.9996 | 0.9996 | 1.0000 | 0.0836 | 0.9995 | 0.9995 | 1 |
| rs192798269 | SNV | 0.1542 | 0.1556 | 0.4661 | 0.1469 | 0.0882 | 0.3054 | 0.3571 | 0.3209 | 0.3118 | 0.3517 | 0.0578 | 0.0035 | 1 |
| rs7073610 | SNV | 0.8620 | 0.1095 | 0.6220 | 0.5664 | 0.5491 | 0.4777 | 0.4550 | 0.0057 | 0.9927 | 0.8595 | 0.8429 | 0.1714 | 1 |
| rs2228507 | SNV | 0.1152 | 0.4216 | 0.0605 | 0.1544 | 0.2312 | 0.0521 | 0.3594 | 0.8435 | 0.0000 | 0.0908 | 0.0564 | 0.1719 | 1 |
| rs139397709 | SNV | 0.5802 | 0.0880 | 0.4435 | 0.6893 | 0.6467 | 0.6912 | 0.8032 | 0.1548 | 0.2221 | 0.6328 | 0.0687 | 0.0063 | 1 |
| rs220126 | SNV | 0.6672 | 0.3769 | 0.2832 | 0.5038 | 0.8141 | 0.2469 | 0.0841 | 0.5931 | 0.1543 | 0.1782 | 0.6029 | 0.0043 | 1 |
| rs2839223 | SNV | 0.4821 | 0.1885 | 0.5567 | 0.8570 | 0.9413 | 0.3744 | 0.2206 | 0.8845 | 0.1137 | 0.5649 | 0.1207 | 0.0004 | 1 |
| HLA-B*13:01 | HLA | 0.1011 | 0.0943 | 0.1173 | 0.1367 | 0.1918 | 0.3448 | 0.0053 | 0.9874 | 0.4638 | 0.1745 | 0.2445 | 0.5219 | 1 |
| chr22:40081915-40082662 | CNV | 0.0052 | 1.0000 | 0.1232 | | 0.9988 | 0.1062 | 0.9996 | 0.9993 | 1.0000 | 0.0557 | 0.9995 | 0.9995 | 1 |
| rs7874348 | SNV | 0.0033 | 0.7797 | 0.4139 | 0.5011 | 0.2914 | 0.7234 | 0.6975 | 0.9682 | 0.8459 | 0.3470 | 0.3532 | 0.3335 | 1 |
| rs3750898 | SNV | 0.3427 | 0.1040 | 0.3800 | 0.2856 | 0.3929 | 0.2519 | 0.3469 | 0.2122 | 0.1265 | 0.9398 | 0.2889 | 0.0007 | 1 |
| chr7:6012757-6013101 | CNV | 0.0702 | 0.5198 | 0.0085 | 0.0936 | 0.2851 | 0.7404 | 0.2005 | 0.2962 | 0.4742 | 0.4031 | 0.3230 | 0.7271 | 1 |
| chr7:5942016-5942639 | CNV | 0 0541 | 0.8850 | 0.1436 | 0.9951 | 0.3677 | 0.0839 | 0.3650 | 0.8725 | 0.2097 | 0.0066 | 0.4245 | 0.4973 | 1 |
| rs1476859 | SNV | 0.0022 | 0.4755 | 0.1647 | 0.3396 | 0.2802 | 0.2401 | 0.5773 | 0.7920 | 0.7623 | 0.0847 | 0.1284 | 0.9569 | 1 |
| rs117457580 | SNV | 0.1842 | 0.4156 | 0.6225 | 0.0072 | 0.0670 | 0.2744 | 0.2383 | 0.8635 | 0.0636 | 0.1620 | 0.0647 | 0.3762 | 1 |
| rs184789735 | SNV | 0.7144 | 0.1341 | 0.7658 | 0.6622 | 0.3866 | 0.4217 | 0.6260 | 0.0041 | 0.1675 | 0.9284 | 0.2279 | 0.5533 | 1 |
| rs1127102 | SNV | 0.5483 | 0.2332 | 0.2379 | 0.1593 | 0.2009 | 0.2495 | 0.8723 | 0.0111 | 0.4846 | 0.7333 | 0.6408 | 0.6323 | 1 |
| chr6:27834340-27834967 | CNV | 0.0098 | 0.9991 | 0.1586 | 0.9998 | 0.9970 | 0.2213 | 1.0000 | 0.9999 | 0.9940 | 0.1028 | 1.0000 | 0.9999 | 1 |
| rs259956 | SNV | 0.0032 | 0.7568 | 0.3315 | 0.3838 | 0.2737 | 0.7052 | 0.3947 | 0.6970 | 0.7441 | 0.3822 | 0.4655 | 0.3135 | 1 |
| rs2421013 | SNV | 0 0013 | 0 4068 | 0 1843 | 0 1927 | 0.1067 | 0 6259 | 0.8162 | 0.9265 | 0 7709 | 0 2660 | 0 3034 | 0.6814 | 1 |
| chr4:15005553-15006221 | CNV | 0.0096 | 0.9983 | 0.0869 | 1.0000 | 0.9998 | 0.2318 | 1.0000 | 1.0000 | 0.9977 | 0.1071 | 1.0000 | 1.0000 | 1 |
| rs10265083 | SNV | 0.4016 | 0.3276 | 0.5948 | 0.9212 | 0.7456 | 0.0070 | 0.1455 | 0.8540 | 0.5208 | 0.1410 | 0.1824 | 0.1820 | 1 |
| rs4740164 | SNV | 0.0801 | 0.0073 | 0.1259 | 0.3713 | 0.2041 | 0.2358 | 0.1451 | 0.1603 | 0.2943 | 0.1778 | 0.2790 | 0.4631 | 1 |
| rs113908376 | SNV | 0.4017 | 0.0522 | 0.9843 | 0.0765 | 0.0778 | 0.6757 | 0.3555 | 0.2742 | 0.3336 | 0.9597 | 0.3408 | 0.0054 | 1 |
| rs3213706 | SNV | 0.7529 | 0.3739 | 0.0555 | 0.6766 | 0.6983 | 0.5523 | 0.0035 | 0.2799 | 0.1768 | 0.8379 | 0.2273 | 0.3336 | 1 |
| rs3740710 | SNV | 0.0659 | 0.6162 | 0.2344 | 0.3022 | 0.0919 | 0.0570 | 0.7520 | 0 8721 | 0.0142 | 0.0502 | 0.2146 | 0.7733 | 1 |
| rs10488698 | SNV | 0.2749 | 0.2474 | 0.0816 | 0.0564 | 0.1018 | 0.6726 | 0.4030 | 0.0036 | 0.7124 | 0.6532 | 0.4607 | 0.5904 | 1 |
| rs1056104 | SNV | 0.2087 | 0.1827 | 0.1594 | 0.3940 | 0.7113 | 0.3524 | 0.9657 | 0.1912 | 0.1446 | 0.0083 | 0.2884 | 0.3505 | 1 |
| rs12099177 | SNV | 0.5387 | 0.4558 | 0.1811 | 0.2856 | 0.4913 | 0.0078 | 0.2650 | 0.9864 | 0.6163 | 0.0822 | 0.3814 | 0.1668 | 1 |
| rs1056827 | SNV | 0.4644 | 0.0572 | 0.1209 | 0.4475 | 0.8391 | 0.0855 | 0.3147 | 0.0159 | 0.9746 | 0.2250 | 0.4532 | 0.1517 | 1 |
| rs1133400 | SNV | 0.1618 | 0.4802 | 0.2832 | 0.7594 | 0.9505 | 0.9845 | 0.1945 | 0.6640 | 0.2136 | 0.5677 | 0.7916 | 0.0044 | 1 |
| rs17417407 | SNV | 0.2346 | 0.2530 | 0.2804 | 0.6026 | 0.5656 | 0.1117 | 0.7173 | 0.0053 | 0.6598 | 0.1552 | 0.1020 | 0.2155 | 1 |
| rs36072133 | SNV | 0.4584 | 0.8289 | 0.7981 | 0.4898 | 0.6505 | 0.8558 | 0.6010 | 0.6533 | 0.0110 | 0.6645 | 0.5622 | 0.1807 | 1 |
| rs4406360 | SNV | 0.1242 | 0.0014 | 0.8958 | 0.1124 | 0.2013 | 0.9353 | 0.0687 | 0.1660 | 0.2518 | 0.8420 | 0.1490 | 0.4973 | 1 |
| rs2641563 | SNV | 0.0017 | 0.3434 | 0.2810 | 0.3250 | 0.1866 | 0.3124 | 0.8228 | 0.8467 | 0.7072 | 0.1330 | 0.2525 | 0.8232 | 1 |
| rs4656077 | SNV | 0.6662 | 0.1401 | 0.8659 | 0.2225 | 0.6660 | 0.4699 | 0.8713 | 0.0053 | 0.1342 | 0.6211 | 0.5586 | 0.9681 | 1 |
| rs11784175 | SNV | 0.4137 | 0.0112 | 0.5899 | 0.9609 | 0.7703 | 0.3144 | 0.9320 | 0.0618 | 0.7940 | 0.9764 | 0.1516 | 0.1461 | 1 |
| chr22:25436575-25437791 | CNV | 0.3548 | 0.0054 | 0.3969 | 0.2947 | 0.5265 | 0.4592 | 0.7562 | 0.3382 | 0.1894 | 0.4353 | 0.1415 | 0.1842 | 1 |
| rs16023 | SNV | 0.6087 | 0.6678 | 0.4706 | 0.9642 | 0.7178 | 0.8122 | 0.5429 | 0.0287 | 0.9107 | 0.4049 | 0.4902 | 0.9931 | 1 |
| rs16889330 | SNV | 0.0571 | 0.8416 | 0.0501 | 0.3986 | 0.2168 | 0.9071 | 0.0055 | 0.9992 | 0.1054 | 0.1581 | 0.1960 | 0.5664 | 1 |
| rs12379 | SNV | 0.0016 | 0.3240 | 0.3301 | 0.1548 | 0.0855 | 0.2900 | 0.4145 | 0.8168 | 0.6528 | 0.1142 | 0.2348 | 0.8599 | 1 |
| rs2913783 | SNV | 0.4139 | 0.5552 | 0.2966 | 0.9488 | 0.9521 | 0.6255 | 0.0032 | 0.7831 | 0.2365 | 0.5859 | 0.2291 | 0.4144 | 1 |
| rs6795970 | SNV | 0.0019 | 0.5582 | 0.1831 | 0.5306 | 0.3224 | 0.5065 | 0.6728 | 0.9027 | 0.9485 | 0.2278 | 0.2646 | 0.5268 | 1 |
| rs11668530 | SNV | 0.6173 | 0.1279 | 0.7152 | 0.8656 | 0.5884 | 0.0080 | 0.4228 | 0.5402 | 0.4924 | 0.2139 | 0.4591 | 0.2627 | 1 |
| rs3743824 | SNV | 0.0032 | 0.4721 | 0.2535 | 0.3510 | 0.1823 | 0.6753 | 0.4622 | 0.7405 | 0.8100 | 0.2703 | 0.4882 | 0.4948 | 1 |
| rs1126464 | SNV | 0.4062 | 0.5038 | 0.3640 | 0.8830 | 0.5806 | 0.6059 | 0.6659 | 0.0138 | 0.4600 | 0.4238 | 0.4973 | 0.8361 | 1 |
| rs2287354 | SNV | 0 5915 | 0.6538 | 0 3121 | 0.4747 | 0.6068 | 0.5546 | 0.4876 | 0.5478 | 0.5514 | 0 2566 | 0.3136 | 0.0024 | 1 |
| rs897945 | SNV | 0.8193 | 0.4366 | 0.8916 | 0.4710 | 0.5386 | 0.1451 | 0.3727 | 0.1541 | 0.9952 | 0.3040 | 0.0775 | 0.0039 | 1 |
| rs2972040 | SNV | 0.5543 | 0.2607 | 0.6373 | 0.2092 | 0.2320 | 0.5928 | 0.4307 | 0.1935 | 0.1475 | 0.7672 | 0.1817 | 0.0007 | 1 |
| rs1678868 | SNV | 0.4506 | 0.5956 | 0.4521 | 0.2559 | 0.6474 | 0.5961 | 0.7262 | 0.0058 | 0.0515 | 0.8912 | 0.7330 | 0.8017 | 1 |
| rs75977701 | SNV | 0.0763 | 0.0847 | 0.4305 | 0.6403 | 0.6202 | 0.0080 | 0.8160 | 0.1530 | 0.0628 | 0.0514 | 0.1151 | 0.1919 | 1 |
| rs11555037 | SNV | 0.5347 | 0.1258 | 0.4781 | 0.6077 | 0.6034 | 0.1391 | 0.7065 | 0.0370 | 0.622$ | 0.1889 | 0.2431 | 0.0504 | 1 |
| rs61978649 | SNV | 0.0016 | 0.4932 | 0.1593 | 0.3988 | 0.2525 | 0.4391 | 0.5601 | 0.9156 | 0.9594 | 0.1898 | 0.2370 | 0.5071 | 1 |
| rs25640 | SNV | 0.2524 | 0.8653 | 0.2897 | 0.2194 | 0.0508 | 0.0079 | 0.9121 | 0.5285 | 0.3933 | 0.0549 | 0.2533 | 0.8462 | 1 |
| rs10513762 | SNV | 0.1935 | 0.4827 | 0.7725 | 0.6165 | 0.2244 | 0.0081 | 0.9248 | 0.4282 | 0.1767 | 0.0981 | 0.1165 | 0.7011 | 1 |
| rs7528153 | SNV | 0.1402 | 0.0047 | 0.9391 | 0.3709 | 0.0921 | 0.2236 | 0.5235 | 0.3817 | 0.2991 | 0.0872 | 0.1099 | 0.1688 | 1 |
| rs4701997 | SNV | 0.1037 | 0.9607 | 0.3339 | 0.3153 | 0.1407 | 0.0544 | 0.1980 | 0.2989 | 0.9967 | 0.0089 | 0.6373 | 0.8850 | 1 |
| rs4546904 | SNV | 0.4150 | 0.4762 | 0.5095 | 0.6626 | 0.8641 | 0.5893 | 0.0012 | 0.2425 | 0.3257 | 0.5620 | 0.4871 | 0.3681 | 1 |
| rs10075302 | SNV | 0.2760 | 0.2223 | 0.7209 | 0.8562 | 0.6456 | 0.7514 | 0.4275 | 0.7890 | 0.0055 | 0.5096 | 0.6562 | 0.9126 | 1 |
| rs11857527 | SNV | 0.2265 | 0.3703 | 0.0936 | 0.4028 | 0.2968 | 0.1490 | | 0.9145 | 0.4245 | 0.7796 | 0.1887 | 0.0022 | 1 |
| rs11954652 | SNV | 0.3725 | 0.2825 | 0.0514 | 0.1658 | 0.2023 | 0.3932 | 0.3397 | 0.0176 | 0.4688 | 0.5151 | 0.7622 | 0.2822 | 1 |
| rs4801798 | SNV | 0.6205 | 0.1382 | 0.8472 | 0.1183 | 0.6424 | 0.4777 | 0.8059 | 0.2229 | 0.4768 | 0.9814 | 0.5782 | 0.0012 | 1 |
| rs2276887 | SNV | 0.1108 | 0.3303 | 0.9613 | 0.0117 | 0.1152 | 0.1480 | 0.0950 | 0.4290 | 0.3556 | 0.4428 | 0.5963 | 0.9369 | 1 |
| rs78112585 | SNV | 0.4347 | 0.4349 | 0.5790 | 0.2023 | 0.5109 | 0.0862 | 0.8393 | 0.0061 | 0.6969 | 0.1564 | 0.1503 | 0.6902 | 1 |
| rs3204850 | SNV | 0 9370 | 0 3395 | 0.4475 | 0 5647 | 0.2222 | 0 8042 | 0.8754 | 0.0043 | 0 9266 | 0 6956 | 0 9010 | 0 3360 | 1 |
| rs4078115 | SNV | 0.8571 | 0.1813 | 0.1330 | 0.7667 | 0.5039 | 0.7111 | 0.2164 | 0.4866 | 0.1785 | 0.8727 | 0.0856 | 0.0039 | 1 |
| rs144388432 | SNV | 0.0562 | 0.4101 | 0.0032 | 0.7420 | 0.7479 | 0.8936 | 0.8985 | 0.5170 | 0.6877 | 0.4236 | 0.1219 | 0.0698 | 1 |
| rs2229902 | SNV | 0.1504 | 0.5876 | 0.1086 | 0.1252 | 0.0094 | 0.7350 | 0.8587 | 0.5734 | 0.7779 | 0.8421 | 0.2592 | 0.9494 | 1 |
| rs3826593 | SNV | 0.2967 | 0.2878 | 0.8350 | 0.7148 | 0.6907 | 0.8561 | 0.1249 | 0.5124 | 0.2216 | 0.8851 | 0.4801 | 0.0010 | 1 |
| rs3809977 | SNV | 0.6272 | 0.1326 | 0.6354 | 0.6026 | 0.8131 | 0.4930 | 0.0768 | 0.1082 | 0.1804 | 0.8571 | 0.3528 | 0.0006 | 1 |
| rs2287679 | SNV | 0.8791 | 0.4280 | 0.6507 | 0.3802 | 0.5628 | 0.6210 | 0.9714 | 0.5212 | 0.8684 | 0.2909 | 0.4171 | 0.0015 | 1 |
| rs76996680 | SNV | 0.3193 | 0.8158 | 0.0848 | 0.0083 | 0.0523 | 0.4937 | 0.3211 | 0.9981 | 0.1583 | 0.2204 | 0.1673 | 0.0813 | 1 |
| rs1345658 | SNV | 0.6205 | 0.1611 | 0.5074 | 0.2561 | 0.5191 | 0.2784 | 0.0612 | 0.1869 | 0.2223 | 0.1657 | 0.3741 | 0.0007 | 1 |
| rs2270518 | SNV | 0.3842 | 0.9417 | 0.5784 | 0.7558 | 0.8623 | 0.5768 | 0.6423 | 0.0100 | 0.4131 | 0.2176 | 0.5778 | 0.4837 | 1 |
| rs3736265 | SNV | 0.1872 | 0.0524 | 0.4575 | 0.4895 | 0.1857 | 0.1250 | 0.8127 | 0.0090 | 0.8408 | 0.4651 | 0.3271 | 0.0854 | 1 |
| rs4696354 | SNV | 0.8426 | 0.1114 | 0.0795 | 0.8581 | 0.9935 | 0.6735 | 0.2991 | 0.3654 | 0.0606 | 0.7938 | 0.5391 | 0.0054 | 1 |
| rs1667354 | SNV | 0.3961 | 0.9042 | 0.3984 | 0.1877 | 0.0062 | 0.8140 | 0.4976 | 0.5521 | 0.2585 | 0.9669 | 0.5209 | 0.4456 | 1 |
| rs56281742 | SNV | 0.0829 | 0.0746 | 0.2404 | 0.6077 | 0.1129 | 0.7834 | 0.6988 | 0.1536 | 0.0104 | 0.8797 | 0.2183 | 0.1541 | 1 |
| rs28725418 | SNV | 0.1698 | 0.0135 | 0.5786 | 0.2974 | 0.1867 | 0.0952 | 0.1097 | 0.1483 | 0.2419 | 0.1295 | 0.3281 | 0.6457 | 1 |
| rs45437199 | SNV | 0.6851 | 0.0807 | 0.2908 | 0.1950 | 0.4387 | 0.9454 | 0.9956 | 0.0086 | 0.2160 | 0.6259 | 0.4755 | 0.6407 | 1 |
| rs1380657 | SNV | 0.0011 | 0.5748 | 0.1973 | 0.2996 | 0.1848 | 0.5248 | 0.5914 | 0.8651 | 0.9954 | 0.2449 | 0.2837 | 0.5067 | 1 |
| rs2278868 | SNV | 0.8004 | 0.1537 | 0.6939 | 0.2873 | 0.3887 | 0.1928 | 0.1722 | 0.1784 | 0.0760 | 0.4323 | 0.1479 | 0.0027 | 1 |
| rs138758776 | SNV | 0.4965 | 0.7052 | 0.3264 | 0.2252 | 0.3443 | 0.1434 | 0.0029 | 0.7919 | 0.1017 | 0.2067 | 0.7000 | 0.5527 | 1 |
| rs25901 | SNV | 0.0014 | 0.3000 | 0.3718 | 0.3960 | 0.2761 | 0.4286 | 0.6932 | 0.9669 | 0.3398 | 0.1208 | 0.2175 | 0.8520 | 1 |
| chr14:106207878-106208450 | CNV | 0.3329 | 0.4413 | 0.3836 | 0.5509 | 0.6796 | 0.1599 | 0.1473 | 0.5928 | 0.2838 | 0.3071 | 0.2210 | 0.0066 | 1 |
| rs670741 | SNV | 0.0771 | 0.1791 | 0.4539 | 0.3593 | 0.4671 | 0.3898 | 0.8927 | 0.3714 | 0.1978 | 0.4242 | 0.9696 | 0.0051 | 1 |
| rs2282302 | SNV | 0.3824 | 0.3234 | 0.3659 | 0.9430 | 0.7653 | 0.2916 | 0.4970 | 0.0070 | 0.6627 | 0.2097 | 0.6262 | 0.2973 | 1 |
| rs45567233 | SNV | 0.4721 | 0.8489 | 0.2080 | 0.2558 | 0.2884 | 0.5271 | 0.0025 | 0.7702 | 0.2826 | 0.6471 | 0.3490 | 0.6284 | 1 |
| rs2295322 | SNV | 0.9310 | 0.8091 | 0.7130 | 0.7511 | 0.5779 | 0.6091 | 0.7945 | 0.0143 | 0.8543 | 0.6859 | 0.3175 | 0.4946 | 1 |
| rs11240573 | SNV | 0.5663 | 0.5090 | 0.2540 | 0.2064 | 0.3327 | 0.7559 | 0.4761 | 0.0105 | 0.9389 | 0.5623 | 0.7098 | 0.5877 | 1 |
| rs909545 | SNV | 0.0029 | 0.6218 | 0.2668 | 0.2804 | 0.2040 | 0.1847 | 0.5002 | 0.9127 | 0.4489 | 0.0855 | 0.3143 | 0.5252 | 1 |
| rs1263872 | SNV | 0.0020 | 0.7886 | 0.2820 | 0.2230 | 0.1414 | 0.7345 | 0.6777 | 0.9911 | 0.7517 | 0.3656 | 0.3905 | 0.3039 | 1 |
| rs2936051 | SNV | 0.8660 | 0.9976 | 0.6815 | 0.8265 | 0.7724 | 0.1052 | 0.0035 | 0.9443 | 0.0950 | 0.3333 | 0.2040 | 0.7166 | 1 |
| rs941952 | SNV | 0.0010 | 0.3434 | 0.1671 | 0.3350 | 0.1747 | 0.3019 | 0.8874 | 0.8636 | 0.6707 | 0.1123 | 0.1291 | 0.7934 | 1 |
| rs183831390 | SNV | 0.3933 | 0.0151 | 0.4713 | 0.9855 | 0.8817 | 0.9386 | 0.5533 | 0.1257 | 0.4635 | 0.9508 | 0.2938 | 0.2390 | 1 |
| rs9567280 | SNV | 0.4307 | 0.6386 | 0.0941 | 0.7178 | 0.7987 | 0.7090 | 0.0162 | 0.2264 | 0.6400 | 0.6550 | 0.4053 | 0.5765 | 1 |
| rs8111625 | SNV | 0.0013 | 0.5761 | 0.2079 | 0.2749 | 0.1823 | 0.5286 | 0.5294 | 0.8508 | 0.9621 | 0.2559 | 0.3021 | 0.4823 | 1 |
| rs2075995 | SNV | 0.3718 | 0.6804 | 0.8318 | 0.4072 | 0.9258 | 0.8822 | 0.0017 | 0.5157 | 0.6524 | 0.3535 | 0.5996 | 0.6744 | 1 |
| rs189467208 | SNV | 0.4969 | 0.1129 | 0.6691 | 0.5408 | 0.6869 | 0.5930 | 0.9998 | 0.0087 | 0.3944 | 0.7751 | 0.6705 | 0.2281 | 1 |
| rs1050112 | SNV | 0.3113 | 0.4596 | 0.5200 | 0.4981 | 0.7280 | 0.9939 | 0.9616 | 0.0115 | 0.7258 | 0.9831 | 0.5792 | 0.5490 | 1 |
| rs115445569 | SNV | 0.6016 | 0.1453 | 0.7214 | 0.1685 | 0.4022 | 0.4784 | 0.9824 | 0.0017 | 0.9049 | 0.7594 | 0.6521 | 0.4394 | 1 |
| rs2240871 | SNV | 0.6479 | 0.6098 | 0.8900 | 0.1933 | 0.1454 | 0.2204 | 0.9842 | 0.8187 | 0.4812 | 0.3300 | 0.2729 | 0.0027 | 1 |
| rs34710081 | SNY | 0.2581 | 0.5194 | 0.4776 | 0.1468 | 0.3521 | 0.0013 | 0.6417 | 0.9653 | 0.3341 | 0.2817 | 0.1876 | 0.2325 | 1 |
| rs2229357 | SNV | 0.2315 | 0.1368 | 0.5802 | 0.6326 | 0.4246 | 0.0841 | 0.0522 | 0.1618 | 0.0077 | 0.1164 | 0.1101 | 0.1649 | 1 |
| rs2297336 | SNV | 0.7811 | 0.4076 | 0.5344 | 0.4094 | 0.9206 | 0.0053 | 0.5131 | 0.1146 | 0.3148 | 0.1938 | 0.5826 | 0.5679 | 1 |
| rs11170877 | SNV | 0.0594 | 0.0533 | 0.1969 | 0.5429 | 0.2364 | 0.3948 | 0.3374 | 0.0039 | 0.1651 | 0.3782 | 0.7486 | 0.4155 | 1 |
| rs9479297 | SNV | 0.0014 | 0.2494 | 0.3871 | 0.4942 | 0.2514 | 0.4408 | 0.6428 | 0.7017 | 0.8364 | 0.1618 | 0.4985 | 0.9615 | 1 |
| rs6911096 | SNV | 0.0033 | 0.5114 | 0.6028 | 0.4942 | 0.2514 | 0.4408 | 0.6428 | 0.7017 | 0.8364 | 0.1618 | 0.4985 | 0.9615 | 1 |
| rs79654965 | SNV | 0.3950 | 0.1722 | 0.6814 | 0.8058 | 0.8254 | 0.1724 | 0.0033 | 0.1907 | 0.1536 | 0.2572 | 0.2607 | 0.0870 | 1 |
| rs77062647 | SNV | 0.1820 | 0.4405 | 0.2138 | 0.5781 | 0.3650 | 0.2288 | 0.6578 | 0.8204 | 0.0097 | 0.0950 | 0.0913 | 0.1209 | 1 |
| rs2043112 | SNV | 0.7707 | 0.1409 | 0.5214 | 0.5315 | 0.3502 | 0.2520 | 0.5038 | 0.0021 | 0.8856 | 0.7507 | 0.2441 | 0.4373 | 1 |
| rs3743315 | SNV | 0.1138 | 0.3537 | 0.0694 | 0.4861 | 0.3214 | 0.6296 | 0.0534 | 0.7355 | 0.0073 | 0.3559 | 0.0651 | 0.2400 | 1 |
| rs3865014 | SNV | 0.0033 | 0.0793 | 0.2262 | 0.1019 | 0.0708 | 0.2001 | 0.7509 | 0.1950 | 0.7153 | 0.0803 | 0.1371 | 0.8419 | 1 |
| rs11071990 | SNV | 0.8526 | 0.0070 | 0.9035 | 0.8008 | 0.6293 | 0.9487 | 0.7864 | 0.2791 | 0.5220 | 0.5992 | 0.5289 | 0.2613 | 1 |
| rs77673307 | SNV | 0.2790 | 0.0773 | 0.5844 | 1.0000 | 0.5587 | 0.3219 | 0.0118 | 0.1478 | 0.2376 | 0.1062 | 0.1183 | 0.0990 | 1 |
| rs3803412 | SNV | 0.8161 | 0.3071 | 0.7410 | 0.9641 | 0.7263 | 0.6004 | 0.9536 | 0.0122 | 0.4021 | 0.8753 | 0.6769 | 0.5001 | 1 |
| rs8040712 | SNV | 0.1704 | 0.3481 | 0.0017 | 0.1113 | 0.0826 | 0.9436 | 0.0782 | 0.8284 | 0.2726 | 0.6382 | 0.1364 | 0.5536 | 1 |
| rs33204 | SNV | 0.5340 | 0.4960 | 0.4960 | 0.5172 | 0.8322 | 0.8176 | 0.0041 | 0.1807 | 0.1945 | 0.9253 | 0.6190 | 0.3137 | 1 |
| rs1801394 | SNV | 0.7589 | 0.4129 | 0.8700 | 0.5105 | 0.5620 | 0.3147 | 0.6475 | 0.3655 | 0.5910 | 0.5467 | 0.7175 | 0.0003 | 1 |
| rs61732315 | SNV | 0.7996 | 0.0533 | 0.9049 | 0.6143 | 0.5590 | 0.9599 | 0.1633 | 0.0312 | 0.0966 | 0.9603 | 0.6639 | 0.2875 | 1 |
| rs3751593 | SNV | 0.9299 | 0.1812 | 0.8235 | 0.9875 | 0.8501 | 0.5405 | 0.2911 | 0.0390 | 0.5660 | 0.3898 | 0.9939 | 0.7509 | 1 |
| rs1864183 | SNV | 0.0027 | 0.7124 | 0.2855 | 0.4291 | 0.2812 | 0.6694 | 0.4929 | 0.7422 | 0.8562 | 0.3553 | 0.3976 | 0.4192 | 1 |
| rs6934505 | SNV | 0.1927 | 0.0595 | 0.4186 | 0.0052 | 0.0734 | 0.4481 | 0.3255 | 0.4379 | 0.5603 | 0.5559 | 0.6605 | 0.1399 | 1 |
| rs10159204 | SNV | 0 5413 | 0 2120 | 0 7818 | 0 6662 | 0 4863 | 0 1166 | 0 0026 | 0 7886 | 0 3271 | 0 2584 | 0 2637 | 0 1458 | 1 |
| rs28757011 | SNV | 0.4977 | 0.0582 | 0.3381 | 0.3705 | 0.4885 | 0.8063 | 0.8801 | 0.7013 | 0.1762 | 0.4907 | 0.3713 | 0.0061 | 1 |
| rs28633659 | SNV | 0.4094 | 0.7953 | 0.2195 | 0.1476 | 0.2488 | 0.0821 | 0.1021 | 0.9634 | 0.5131 | 0.0775 | 0.0097 | 0.1511 | 1 |
| rs2792205 | SNV | 0.1287 | 0.0072 | 0.5848 | 0.9244 | 0.6224 | 0.2961 | 0.2438 | 0.1292 | 0.5126 | 0.3182 | 0.2465 | 0.4183 | 1 |
| rs4027404 | SNV | 0.6392 | 0.0855 | 0.6738 | 0.1746 | 0.2879 | 0.4985 | 0.5681 | 0.0019 | 0.5831 | 0.7838 | 0.5475 | 0.1638 | 1 |
| rs56107695 | SNV | 0.4029 | 0.5293 | 0.5901 | 0.0033 | 0.0588 | 0.9978 | 0.5432 | 0.6060 | 0.1800 | 0.9569 | 0.1281 | 0.1307 | 1 |

As a result, as shown in FIG. 5B, most of SNV functions were specifically associated with specific irAE types, unlike laboratory data (e.g., NLR) and HLA-B variations (e.g., HLA-B*35:01 and chr6: 31324462-31324741 CNVs). Because the number of irAE cases of each type was significantly lower than a control sample, to evaluate model performance, as shown in FIG. 5C, average precision (i.e., the accurate prediction rate of irAE occurrence) was used. For verification using samples not included in model training, non-interest irAEs were used as a dummy set to perform the prediction of irAE of interest. Verification performance was measured by the accuracy and area under the curve (AUC) of receiver operating characteristic analysis.

To identify the significant SNV affecting each irAE type, the average effect of each function on model prediction was calculated using the Shapley value (Shapley, 1951), and 10 most important SNVs for each model are listed in Table 9 below, and the Shapley values of SNV and CNV for Any are shown in FIG. 5D. In FIG. 5D, red and blue horizontal dotted lines represent P values of 0.005 and 0.05, respectively, and orange and blue dots represent SNV and CNV with P<0.005. Red dots represent the top 10 SNPs with the highest Shapley values in the prediction model. The P values were calculated by multivariate logistic regression analysis using age and sex as covariates.

TABLE 9

| Type of irAE | SNT ID | rsID | mean_abs_shap | stdev_abs_shap | Annotated gene |
|---|---|---|---|---|---|
| Any | chr17_76187108_G_C | rs77585764 | 0.029 | 0.036 | AFMID |
| Any | chr21_37661405_G_A | rs3827183 | 0.028 | 0.009 | DOPEY2 |
| Any | chr19_35719020_C_T | rs541169 | 0.030 | 0.027 | FAM187B |
| Any | chr4_187629770_A_C | rs3733414 | 0.033 | 0.009 | FAT1 |
| Any | chr4_103611845_C_T | rs227368 | 0.035 | 0.018 | MANBA |
| Any | chr11_1972205_G_A | rs2240197 | 0.033 | 0.015 | MRPL23 |
| Any | chr8_52733050_T_A | rs12335014 | 0.039 | 0.014 | PCMTD1 |
| Any | chr16_72156842_T_G | rs16973716 | 0.046 | 0.007 | PMFBP1 |
| Any | chr10_134646988_C_T | rs4880433 | 0.029 | 0.004 | TTC40 |
| Any | chr13_32885654_T_C | rs445909 | 0.032 | 0.012 | ZAR1L |
| Critical | chr10_24908686_T_C | rs3748222 | 0.023 | 0.008 | ARHGAP21 |
| Critical | chr12_13526328_G_T | rs17821405 | 0.024 | 0.017 | C12orf36 |
| Critical | chr13_113909339_A_G | rs2302757 | 0.021 | 0.007 | CUL4A |
| Critical | chr21_37661405_G_A | rs3827183 | 0.021 | 0.012 | DOPEY2 |
| Critical | chr11_73988167_C_T | rs2282488 | 0.028 | 0.004 | P4HA3 |
| Critical | chr18_10731428_C_T | rs2865121 | 0.016 | 0.004 | PIEZO2 |
| Critical | chr8_10390452_C_T | rs4406360 | 0.022 | 0.014 | PRSS55 |
| Critical | chr1_28282292_T_C | rs3813803 | 0.024 | 0.000 | SMPDL3B |
| Critical | chr22_50964236_G_A | rs11479 | 0.023 | 0.000 | TYMP |
| Critical | chr1_108307727_T_A | rs7528153 | 0.017 | 0.006 | VAV3 |
| Endocrine | chr7_150733025_T_A | rs2303926 | 0.025 | 0.001 | ABCBS |
| Endocrine | chr20_31688241_T_C | rs11699009 | 0.029 | 0.002 | BPIFB4 |
| Endocrine | chr21_43523941_C_A | rs220111 | 0.026 | 0.005 | C21orf128 |
| Endocrine | chr8_25364834_A_T | rs3829009 | 0.021 | 0.001 | CDCA2 |
| Endocrine | chr13_50467044_G_C | rs554324677 | 0.021 | 0.008 | CTAGE10P |
| Endocrine | chr6_46563817_A_T | rs7761731 | 0.030 | 0.003 | CYP39A1 |
| Endocrine | chr11_19735987_A_G | rs2278132 | 0.022 | 0.002 | LOC100126784 |
| Endocrine | chr8_52733050_T_A | rs12335014 | 0.026 | 0.013 | PCMTD1 |
| Endocrine | chr20_44141331_A_G | rs6032259 | 0.029 | 0.000 | SPINT3 |
| Endocrine | chr19_56703248_G_A | rs527025 | 0.023 | 0.000 | ZSCAN5B |
| Flu-like | chr4_91230579_G_A | rs12647859 | 0.027 | 0.003 | CCSER1 |
| Flu-like | chr8_25364834_A_T | rs3829009 | 0.031 | 0.001 | CDCA2 |
| Flu-like | chr5_13894894_T_A | rs4701997 | 0.030 | 0.006 | DNAH5 |
| Flu-like | chr2_39008949_G_A | rs1056104 | 0.026 | 0.001 | GEMIN6 |
| Flu-like | chr1_79095581_T_C | rs987495 | 0.028 | 0.004 | IFI44L |
| Flu-like | chr10_22839628_T_C | rs2230469 | 0.028 | 0.014 | PIP4K2A |
| Flu-like | chr11_94322352_A_T | rs11020845 | 0.028 | 0.005 | PIWIL4 |
| Flu-like | chr2_95947099_G_A | rs72819488 | 0.026 | 0.008 | PROM2 |
| Flu-like | chr1_227935762_G_A | rs2236358 | 0.036 | 0.006 | SNAP47 |
| Flu-like | chr17_5058808_G_A | rs9899177 | 0.029 | 0.010 | USP6 |
| Gastrointestinal | chr5_114607217_T_A | rs77125878 | 0.020 | 0.010 | CCDC112 |
| Gastrointestinal | chr2_202939654_T_C | rs10221698 | 0.018 | 0.008 | LOC100652824 |
| Gastrointestinal | chr21_16339570_T_A | rs2228507 | 0.019 | 0.008 | NRIP1 |
| Gastrointestinal | chr19_50412650_C_T | rs3745489 | 0.017 | 0.009 | NUP62 |
| Gastrointestinal | chr12_57619362_G_A | rs10783816 | 0.021 | 0.002 | NXPH4 |
| Gastrointestinal | chr6_144081609_C_T | rs2073214 | 0.032 | 0.008 | PHACTR2 |
| Gastrointestinal | chr10_3200292_G_A | rs3182535 | 0.020 | 0.008 | PITRM1 |
| Gastrointestinal | clu8_26365716_C_T | rs2233701 | 0.023 | 0.003 | PNMA2 |
| Gastrointestinal | chr8_10467589_T_C | rs9657518 | 0.023 | 0.013 | RP1L1 |
| Gastrointestinal | chr13_41382711_A_T | rs17849654 | 0.018 | 0.000 | SLC25A15 |
| Musculoskeletal | chr2_241451351_G_A | rs35044862 | 0.020 | 0.002 | ANKMY1 |
| Musculoskeletal | chr5_131822072_A_G | rs2070724 | 0.018 | 0.003 | C5orf56 |
| Musculoskeletal | chr19_45016116_A_G | rs8100718 | 0.020 | 0.003 | CEACAM20 |
| Musculoskeletal | chr5_118506685_G_A | rs9790916 | 0.017 | 0.004 | DMXL1 |
| Musculoskeletal | chr4_22389797_A_C | rs3814416 | 0.017 | 0.012 | GPR125 |
| Musculoskeletal | chr5_118811533_G_A | rs25640 | 0.022 | 0.004 | HSD17B4 |
| Musculoskeletal | chr19_35719106_A_G | rs565791 | 0.015 | 0.003 | NXPH4 |
| Musculoskeletal | chr1_67109335_A_G | rs7526812 | 0.017 | 0.006 | SGIP1 |
| Musculoskeletal | chr17_7606722_C_G | rs7640 | 0.020 | 0.003 | WRAP53 |
| Musculoskeletal | chr7_64863392_C_T | rs10265083 | 0.019 | 0.006 | ZNF92 |
| Multiple (any grade) | chr4_114278277_C_T | rs3733617 | 0.029 | 0.016 | ANK2 |
| Multiple (any grade) | chr12_48888594_C_T | rs11458 | 0.034 | 0.016 | C12orf54 |
| Multiple (any grade) | chr18_25616451_A_T | rs1944294 | 0.028 | 0.005 | CDH2 |
| Multiple (any grade) | chr19_41117869_A_G | rs1131620 | 0.028 | 0.009 | LTBP4 |
| Multiple (any grade) | chr7_99696797_T_C | rs2070215 | 0.041 | 0.007 | MCM7 |
| Multiple (any grade) | chr12_57619362_G_A | rs10783816 | 0.032 | 0.009 | NXPH4 |
| Multiple (any grade) | chr17_3119767_C_T | rs769427 | 0.032 | 0.016 | OR1A1 |
| Multiple (any grade) | chr6_144081609_C_T | rs2073214 | 0.028 | 0.012 | PHACTR2 |
| Multiple (any grade) | chr12_14720554_G_T | rs1141509 | 0.046 | 0.008 | PLBD1 |
| Multiple (any grade) | chr1_248039451_C_T | rs3811444 | 0.028 | 0.007 | TRIM58 |
| Multiple (grade ≥2) | chr11_134856603_C_T | rs117318814 | 0.017 | 0.000 | AK130852 |
| Multiple (grade ≥2) | chr4_114278277_C_T | rs3733617 | 0.017 | 0.001 | ANK2 |
| Multiple (grade ≥2) | chr2_241451351_G_A | rs35044862 | 0.013 | 0.001 | ANKMY1 |
| Multiple (grade ≥2) | chr18_76829525_A_G | rs4078115 | 0.017 | 0.003 | ATP9B |
| Multiple (grade ≥2) | chr5_114607217_T_A | rs77125878 | 0.014 | 0.011 | CCDC112 |
| Multiple (grade ≥2) | chr5_118506685_G_A | rs9790916 | 0.019 | 0.002 | DMXL1 |
| Multiple (grade ≥2) | chr21_37617724_C_A | rs3746866 | 0.015 | 0.004 | DOPEY2 |
| Multiple (grade ≥2) | chr4_89577214_A_T | rs2972040 | 0.015 | 0.001 | HERC3 |
| Multiple (grade ≥2) | chr5_7870973_A_G | rs1801394 | 0.013 | 0.004 | MTRR |
| Multiple (grade ≥2) | chr11_48238549_G_A | rs11606506 | 0.020 | 0.007 | OR4B1 |
| Neurologic | chr3_100558416_T_C | rs9841585 | 0.016 | 0.005 | ABI3BP |
| Neurologic | chr19_5772897_C_T | rs78536254 | 0.020 | 0.000 | CATSPERD |
| Neurologic | chr5_118506685_G_A | rs9790916 | 0.017 | 0.002 | DMXL1 |
| Neurologic | chr2_234750542_G_C | rs3732215 | 0.023 | 0.002 | HJURP |
| Neurologic | chr1_6157434_C_T | rs2311802 | 0.020 | 0.013 | KCNAB2 |
| Neurologic | chr13_44457925_A_G | rs3764147 | 0.019 | 0.004 | LACC1 |
| Neurologic | chr1_222803204_A_G | rs2936051 | 0.015 | 0.002 | MIA3 |
| Neurologic | chr15_42143533_T_G | rs1197665 | 0.018 | 0.001 | SPTBN5 |
| Neurologic | chr16_2812939_C_A | rs2240140 | 0.016 | 0.006 | SRRM2 |
| Neurologic | chr17_15611495_T_C | rs3760299 | 0.015 | 0.007 | ZNF286A |
| Pulmonary | chr6_109894773_T_A | rs12175588 | 0.004 | 0.002 | AK9 |
| Pulmonary | chr2_228493211_G_A | rs28739019 | 0.004 | 0.001 | C2orf83 |
| Pulmonary | chr14_24679877_C_T | rs2295322 | 0.004 | 0.000 | CHMP4A |
| Pulmonary | chr2_220037666_A_G | rs1127102 | 0.004 | 0.001 | CNPPD1 |
| Pulmonary | chr14_55655692_C_T | rs2274271 | 0.004 | 0.004 | DLGAP5 |
| Pulmonary | chr19_54758504_C_G | rs117301131 | 0.005 | 0.004 | LILRB5 |
| Pulmonary | chr8_125568175_T_C | rs3793395 | 0.004 | 0.002 | MTSS1 |
| Pulmonary | chr14_60585131_A_G | rs308998 | 0.004 | 0.000 | PCNXL4 |
| Pulmonary | chr7_140080087_C_G | rs62490396 | 0.006 | 0.001 | SLC37A3 |
| Pulmonary | chr8_135649848_G_A | rs12541381 | 0.005 | 0.001 | ZFAT |
| Skin | chr7_150733025_T_A | rs2303926 | 0.056 | 0.001 | ABCB8 |
| Skin | chr12_52306221_C_T | rs2277382 | 0.034 | 0.036 | ACVRL1 |
| Skin | chr15_34159986_T_C | rs2141647 | 0.043 | 0.014 | AVEN |
| Skin | chr14_74489735_G_A | rs3784039 | 0.049 | 0.058 | CCDC176 |
| Skin | chr19_35719020_C_T | rs541169 | 0.040 | 0.020 | FAM187B |
| Skin | chr3_128204693_G_C | rs78245253 | 0.035 | 0.043 | GATA2 |
| Skin | chr9_712156_T_G | rs912174 | 0.051 | 0.018 | KANK1 |
| Skin | chr19_51361472_C_A | rs2003783 | 0.039 | 0.025 | KLK3 |
| Skin | chr12_31288955_G_A | rs11051266 | 0.035 | 0.002 | OVOS2 |
| Skin | chr1_227935762_G_A | rs2236358 | 0.051 | 0.009 | SNAP47 |

Particularly, this list was heavily delineated by genes associated with the immune system. For example, DOPEY2 was differentially expressed in CD8+ T cells of ICB responders (Chen, I. X et al., Proc Natl Acad Sci USA. 2020 Sep. 22; 117 (38): 23684-23694.), and MRPL23 is a component of the lncRNA-related signature predicting prognosis after ICB treatment in bladder cancer (Wu, Y et al., Aging (Albany. NY). 12, 23306-23325.). Some genes were associated with autoimmune diseases. For example, MANBA has been reported to be associated with ulcerative colitis (Jostins, L et al., Nature 491, 119-124.), whereas PMFBP1 (Ibanez-Cabellos, J. S et al., Front. Genet. 10, 1104.) and TTC40 (Ham, S et al., Exp. Mol. Med. 51, 1-13.) have been associated with rheumatoid arthritis. Another important gene, AFMID, has been reported to be involved in an immunoregulatory circuit (Proietti, E et al., Trends Immunol. 41, 1037-1050.).

Finally, the most important feature of Any is TMEM162 (also referred to as FAM187B or FLJ25660). Accordingly, in the present invention, the association between variation in TMEM162 gene and irAEs was confirmed in the following Experimental Example.

### Experimental Example 6. Identification of Non-Sense Mutation of TMEM162 as Risk Factor of irAEs

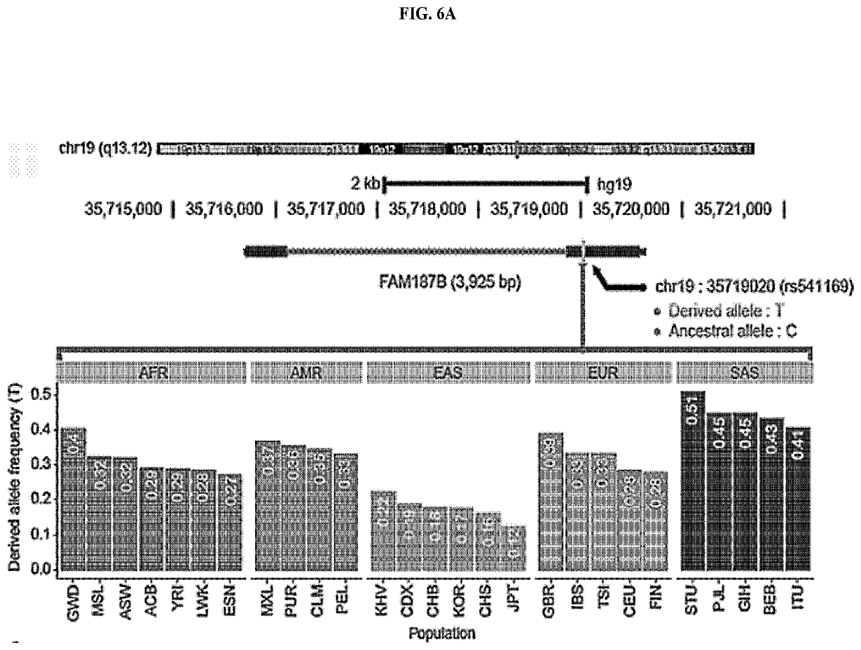

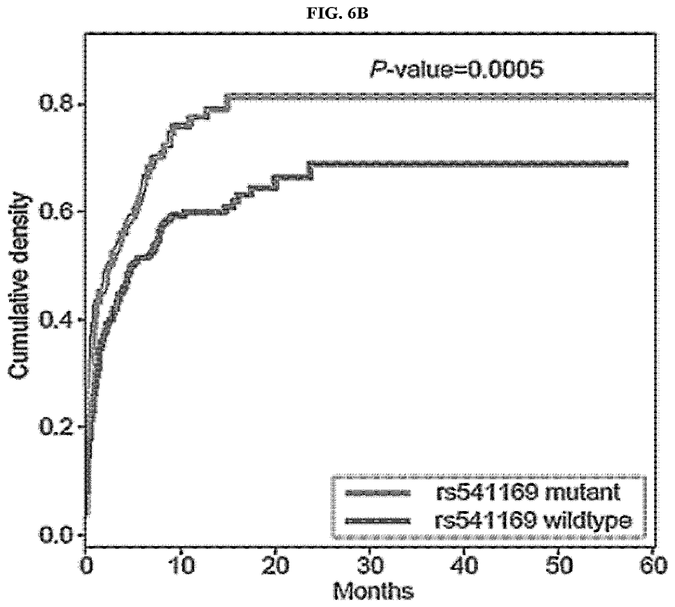

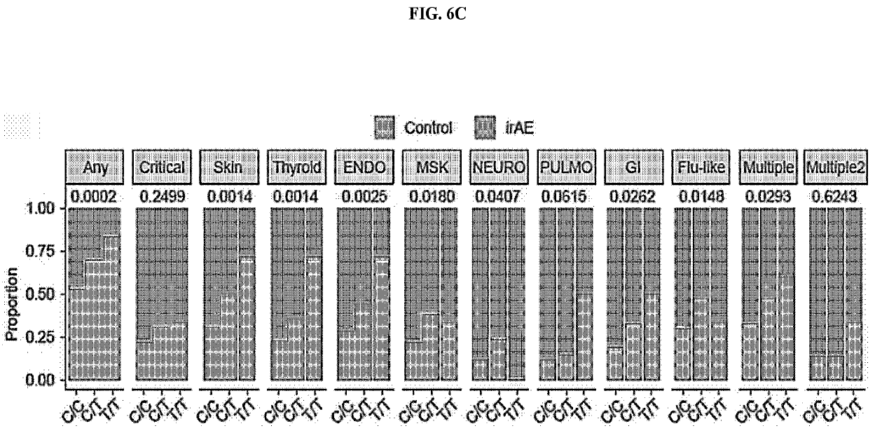

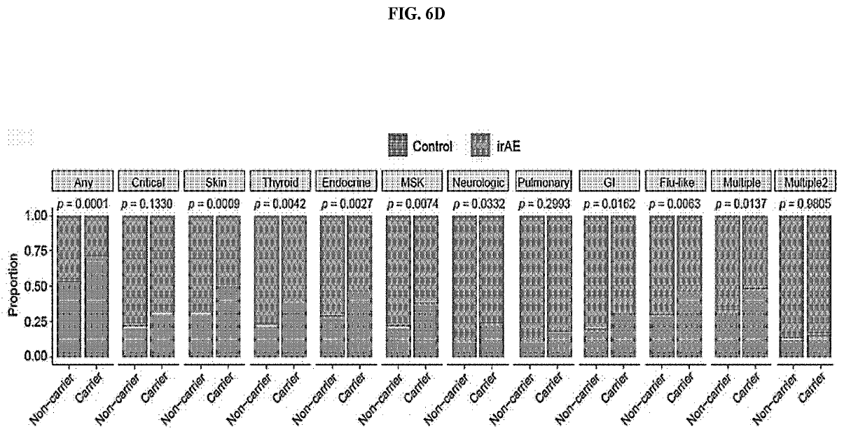

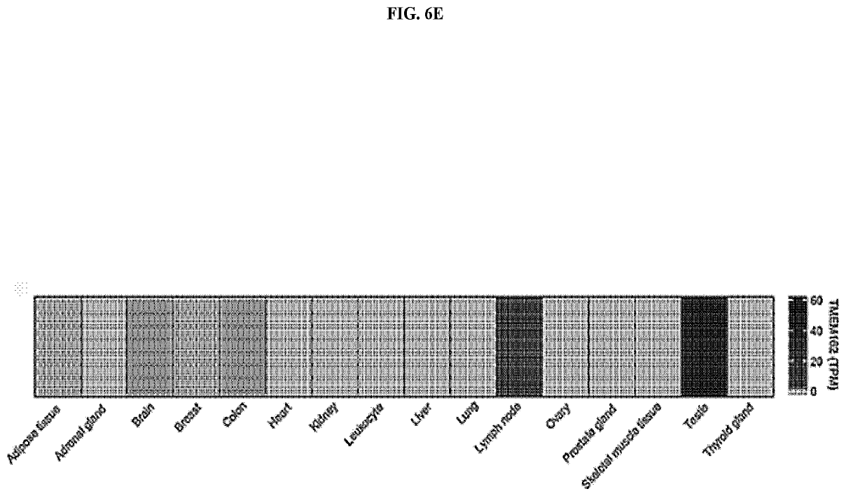

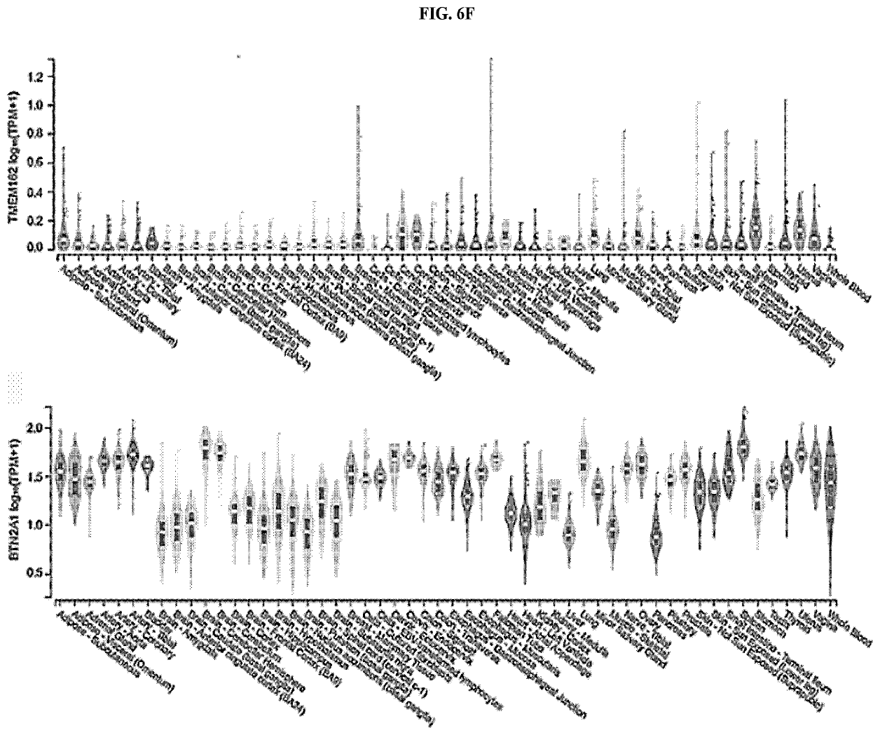

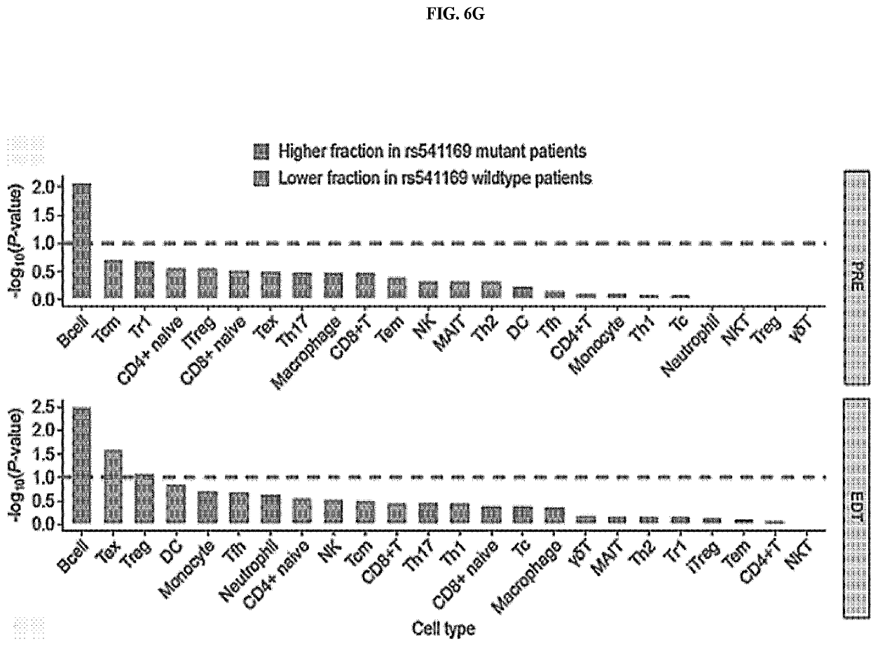

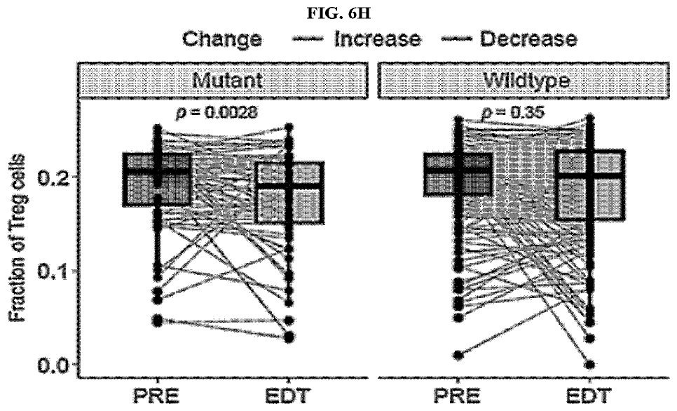

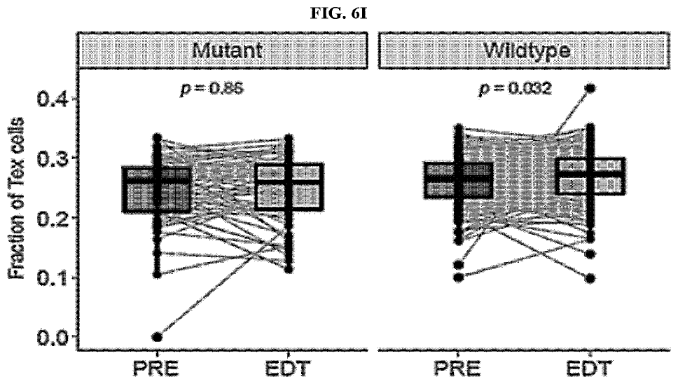

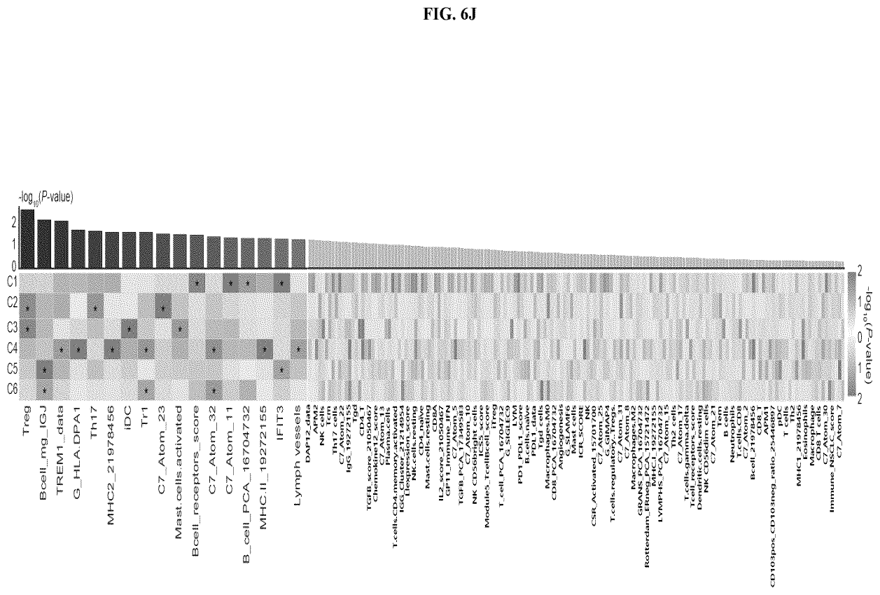

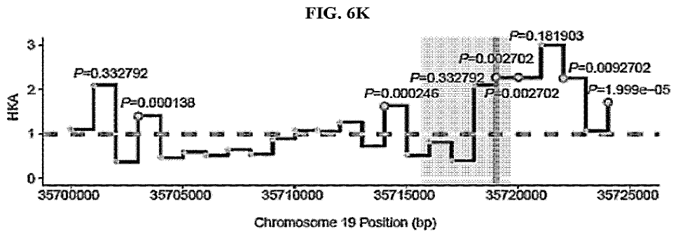

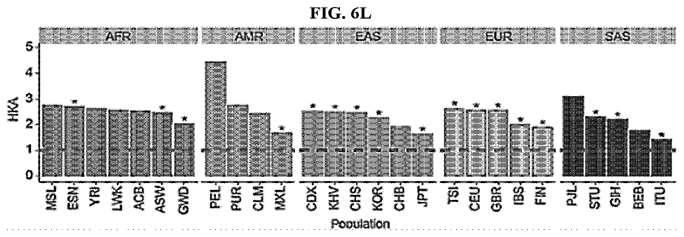

An experiment was conducted focusing on rs541169 (chr19:35719020 C>T) that causes the cleavage of TMEM162 protein due to variation into allele T. This mutation was most significant in one of the top 10 variations having the highest Shapley value for patients with irAEs and Any prediction model (refer to FIG. 5D). As shown in FIG. 6A, the derived allele frequency (DAF) in the cohort of the present invention was 0.14, which is similar to DAF of 0.17 obtained from the 1094 Korean genomes. In addition, continuous incidence analysis using the follow-up period after ICB administration showed that, as shown in FIG. 6B, the time to irAE occurrence is significantly shorter for patients carrying mutation. The frequency spectrum of the rs541169 genotype, as shown in FIG. 6C, has significant differences according to irAE status, along with the fractions of irAE cases increasing according to the copy number of alleles (i.e., C/C, C/T and T/T). The results were similar to those when patients were divided into two groups, a mutation carrier and a non-mutation carrier, as shown in FIG. 6D.

Although the function of immunoglobulin superfamily protein is not well established, a recent systemic cell surface interaction screen determined and further validate the interaction between TMEM162 (FAM187B) and BTN2A1 (Verschueren, E et al., Cell 182, 329-344.e19.). Butyrophilin (BTN) proteins play a role in lymphocyte activation, and various studies suggest the roles of some BTN-based components in autoimmune diseases (Afrache, H et al., Immunogenetics 64, 781-794.). The role of BTN2A1 as an immune checkpoint was recently discovered. In the present invention, as shown in FIGS. 6E and 6F, it was found that TMEM162 is specifically expressed in the lymph node and spleen, and BTN2A1 is also highly expressed in the spleen.

Based on these results, whether a nonsense variation in rs541169 could affect immune cell composition was investigated. Since CBC data support limited cell types, cell fraction inference was performed from whole blood RNA sequencing data for 24 immune cell types. Immune cell type scores were compared between a mutant group (nonsense mutation carriers) and a wild-type (non-carrier) group. As a result, as shown in FIG. 6G, B lymphocyte scores were significantly higher in the mutant group for both PRE and EDT samples. In addition, scores for exhausted T (Tex) cells and regulatory T (Treg) cells were significantly lower among mutations only in the EDT sample, but not in the PRE sample, suggesting differential T cell regulation in response to ICB treatment. In fact, the PRE-EDT comparison revealed, as shown in FIG. 6H, a significant on-therapy decrease in Treg cells only in the mutant group, suggesting that suppressed Treg activity may be responsible for the higher irAE risk in the mutation carrier group. In contrast, as shown in FIG. 6I, only the non-mutant carrier group showed a significant increase in Tex cells. Therefore, it was confirmed that limited T cell exhaustion could be another factor causing irAEs in the mutation carrier group.

To investigate the effect of mutations on tumor immune environment, similar analyses were performed on TCGA pan-cancer samples using various immune signature scores, and the results are shown in FIG. 6J (Thorsson, V et al., Immunity 48, 812-830.e14.). Specifically, the immune signature scores were compared between mutant and wild-type samples grouped into immune clusters, resulting in P values from the Mann-Whitney U tests. In FIG. 6J, red and blue, as well as * that indicates P<0.05, represent higher or lower immune signatures in mutants than those in the wild-type samples, respectively. Immune signatures with P<0.05 in one or more immune clusters are shown enlarged on the left. Each of C to C6 means: C1, would healing; C2, IFN-γ dominant; C3, inflammatory; C4, exhausted lymphocyte; C5, immunological silence; C6, TGF-β dominant.

As a result, as shown in FIG. 6J, similar patterns were observed in Treg and B cells, which best distinguished the mutant and wild-type samples. Based on cohort analysis, TMEM162 mutation decreased Treg infiltration, and increased B cell infiltration. More specifically, the mutation results in significantly fewer Treg cells in C2 (IFN-γ dominant) and C3 (inflammatory) tumors (refer to FIG. 6J). This is consistent with reduced Treg activity in the cohort observed only after ICB treatment (refer to FIG. 6H). Meanwhile, the mutation increased B cells in most immune clusters, but most significantly in C5 (immune silent) and C6 (TGF-β dominant) tumors (refer to FIG. 6J).

By the genome-wide association study for Crohn's disease based on non-synonymous variation, rs541169 was confirmed (Hampe, J et al., Nat. Genet. 39, 207-211.). Increasing evidence shows that the variation of autoimmune disease is a target of natural selection by virtue of its contribution to protection against infection (Ramos, P. S et al., J. Hum. Genet. 60, 657.). For example, a balancing selection signature reflecting the balanced function of opposing alleles in bacterial defense and autoimmune control was detected at the locus of an inflammatory bowel disease (Jostins, L et al., Nature 491, 119-124.). The rs541169 variation was identified as one of the 8 most prominent loss-of-function mutations upon selection, considering the significant level of population differentiation (Rausell, A et al., Proc. Natl. Acad. Sci. 117 (24) 13626-13636), which was also observed in the data of the present invention, including the Korean population (refer to FIG. 6A).

HKA tests (Hudson, R. R et al., Genetics 116, 153-159.) were performed across the chromosomal region surrounding rs541169 using the 1,094 Korean whole genome sequences (Jeon, S et al., Sci. Adv. 6, eaaz7835.), and the results are shown in FIG. 6K. The gray horizontal dashed line in FIG. 6K indicates HKA value 1 as the threshold for supporting balancing selection. The yellow vertical solid line and green shading indicate the positions of rs541169 and TMEM16, respectively. The HKA tests obtained, as shown in FIG. 6K, HKA k>1 (P=0.0027) for the variation, indicating balancing selection operating in the Korean population. When repeated for various populations, the HKA test results showed, as shown in FIG. 6L, HKA k>1 in all investigated populations, suggesting that this locus is under wide-spread balancing selection. In FIG. 6L, the blue horizontal dashed line represents the HKA value 1, which is the threshold supporting balancing selection.

It should be understood by those of ordinary skill in the art that the above descriptions of the present invention are exemplary. Herein, the example embodiments disclosed can be easily modified into other specific forms without changing the technical spirit or essential features of the present invention. Therefore, it should be interpreted that the example embodiments described above are exemplary in all aspects, and are not limitative.

### INDUSTRIAL APPLICABILITY

As rs541169 according to the present invention is expected to be useful as a biomarker for predicting the occurrence of an immunotherapy-induced irAEs or the responsiveness to a cancer immunotherapy, and thus has industrial applicability.

1-14. (canceled)

15. A method, comprising:
detecting a SNP in a dbSNP database rs541169 in a biological sample isolated from a subject;
predicting a high risk of an occurrence of cancer immunotherapy-induced immune-related adverse events (irAEs) when a variation in which a nucleotide of dbSNP database rs541169 is T is detected in the biological sample; and
when the high risk of the occurrence of a cancer immunotherapy-induced irAEs is predicted, treating the cancer immunotherapy-induced irAEs with a corticosteriod or an immunosuppressant.

16. The method of claim 1, wherein the SNP of the rs541169 causes the cleavage of TMEM162 protein.

17. The method of claim 1, wherein the SNP is detected with a detection agent that is a primer or probe able to detect rs541169 SNP.

18. The method of claim 1, wherein the cancer immunotherapy-induced irAEs is one or more selected from the group consisting of a skin adverse event, an endocrine system adverse event, a thyroid gland adverse event, a musculoskeletal system adverse event, a gastrointestinal system adverse event, a neurologic system adverse event, a flu-like symptom, and pneumonia.

19. The method of claim 1, further comprising:
an agent for detecting a SNP of the dbSNP database listed in the following table

| Type of irAE | SNV ID | rsID | mean_abs_shap | stdev_abs_shap | Annotated gene |
|---|---|---|---|---|---|
| Any | chr17_76187108_G_C | rs77585764 | 0.029 | 0.036 | AFMID |
| Any | chr21_37661405_G_A | rs3827183 | 0.028 | 0.009 | DOPEY2 |
| Any | chr4_187629770_A_C | rs3733414 | 0.033 | 0.009 | FAT1 |
| Any | chr4_103611845_C_T | rs227368 | 0.035 | 0.018 | MANBA |
| Any | chr11_1972205_G_A | rs2240197 | 0.033 | 0.015 | MRPL23 |
| Any | chr8_52733050_T_A | rs12335014 | 0.039 | 0.014 | PCMTD1 |
| Any | chr16_72156842_T_G | rs16973716 | 0.046 | 0.007 | PMFBP1 |
| Any | chr10_134646988_C_T | rs4880433 | 0.029 | 0.004 | TTC40 |
| Any | chr13_32885654_T_C | rs445909 | 0.032 | 0.012 | ZAR1L |
| Critical | chr10_24908686_T_C | rs3748222 | 0.023 | 0.008 | ARHGAP21 |
| Critical | chr12_13526328_G_T | rs17821405 | 0.024 | 0.017 | C12orf36 |
| Critical | chr13_113909339_A_G | rs2302757 | 0.021 | 0.007 | CUL4A |
| Critical | chr21_37661405_G_A | rs3827183 | 0.021 | 0.012 | DOPEY2 |
| Critical | chr11_73988167_C_T | rs2282488 | 0.028 | 0.004 | P4HA3 |
| Critical | chr18_10731428_C_T | rs2865121 | 0.016 | 0.004 | PIEZO2 |
| Critical | chr8_10390452_C_T | rs4406360 | 0.022 | 0.014 | PRSS55 |
| Critical | chr1_28282292_T_C | rs3813803 | 0.024 | 0.000 | SMPDL3B |
| Critical | chr22_50964236_G_A | rs11479 | 0.023 | 0.000 | TYMP |
| Critical | chr1_108307727_T_A | rs7528153 | 0.017 | 0.006 | VAV3 |
| Endocrine | chr7_150733025_T_A | rs2303926 | 0.025 | 0.001 | ABCBS |
| Endocrine | chr20_31688241_T_C | rs11699009 | 0.029 | 0.002 | BPIFB4 |
| Endocrine | chr21_43523941_C_A | rs220111 | 0.026 | 0.005 | C21orf128 |
| Endocrine | chr8_25364834_A_T | rs3829009 | 0.021 | 0.001 | CDCA2 |
| Endocrine | chr13 50467044 G C | rs554324677 | 0.021 | 0.008 | CTAGE10P |
| Endocrine | chr6_46563817_A_T | rs7761731 | 0.030 | 0.003 | CYP39A1 |
| Endocrine | chr11_19735987_A_G | rs2278132 | 0.022 | 0.002 | LOC100126784 |
| Endocrine | chr8_52733050_T_A | rs12335014 | 0.026 | 0.013 | PCMTD1 |
| Endocrine | chr20_44141331_A_G | rs6032259 | 0.029 | 0.000 | SPINT3 |
| Endocrine | chr19_56703248_G_A | rs527025 | 0.023 | 0.000 | ZSCAN5B |
| Flu-like | chr4_91230579_G_A | rs12647859 | 0.027 | 0.003 | CCSER1 |
| Flu-like | chr8_25364834_A_T | rs3829009 | 0.031 | 0.001 | CDCA2 |
| Flu-like | chr5_13894894_T_A | rs4701997 | 0.030 | 0.006 | DNAH5 |
| Flu-like | chr2_39008949_G_A | rs1056104 | 0.026 | 0.001 | GEMIN6 |
| Flu-like | chr1_79095581_T_C | rs987495 | 0.028 | 0.004 | IFI44L |
| Flu-like | chr10_22839628_T_C | rs2230469 | 0.028 | 0.014 | PIP4K2A |
| Flu-like | chr11_94322352_A_T | rs11020845 | 0.028 | 0.005 | PIWIL4 |
| Flu-like | chr2_95947099_G_A | rs72819488 | 0.026 | 0.008 | PROM2 |
| Flu-like | chr1_227935762_G_A | rs2236358 | 0.036 | 0.006 | SNAP47 |
| Flu-like | chr17_5058808_G_A | rs9899177 | 0.029 | 0.010 | USP6 |
| Gastrointestinal | chr5_114607217_T_A | rs77125878 | 0.020 | 0.010 | CCDC112 |
| Gastrointestinal | chr2_202939654_T_C | rs10221698 | 0.018 | 0.008 | LOC100652824 |
| Gastrointestinal | chr21_16339570_T_A | rs2228507 | 0.019 | 0.008 | NRIP1 |
| Gastrointestinal | chr19_50412650_C_T | rs3745489 | 0.017 | 0.009 | NUP62 |
| Gastrointestinal | chr12_57619362_G_A | rs10783816 | 0.021 | 0.002 | NXPH4 |
| Gastrointestinal | chr6_144081609_C_T | rs2073214 | 0.032 | 0.008 | PHACTR2 |
| Gastrointestinal | chr10_3200292_G_A | rs3182535 | 0.020 | 0.008 | PITRM1 |
| Gastrointestinal | chr8 26365716 C T | rs2233701 | 0.023 | 0.003 | PNMA2 |
| Gastrointestinal | chr8_10467589_T_C | rs9657518 | 0.023 | 0.013 | RPIL1 |
| Gastrointestinal | chr13_41382711_A_T | rs17849654 | 0.018 | 0.000 | SLC25A15 |
| Musculoskeletal | chr2_241451351_G_A | rs35044862 | 0.020 | 0.002 | ANKMY1 |
| Musculoskeletal | chr5_131822072_A_G | rs2070724 | 0.018 | 0.003 | C5orf56 |
| Musculoskeletal | chr19_45016116_A_G | rs8100718 | 0.020 | 0.003 | CEACAM20 |
| Musculoskeletal | chr5_118506685_G_A | rs9790916 | 0.017 | 0.004 | DMXL1 |
| Musculoskeletal | chr4_22389797_A_C | rs3814416 | 0.017 | 0.012 | GPR125 |
| Musculoskeletal | chr5_118811533_G_A | rs25640 | 0.022 | 0.004 | HSD17B4 |
| Musculoskeletal | chr19_35719106_A_G | rs565791 | 0.015 | 0.003 | NXPH4 |
| Musculoskeletal | chr1_67109335_A_G | rs7526812 | 0.017 | 0.006 | SGIP1 |
| Musculoskeletal | chr17_7606722_C_G | rs7640 | 0.020 | 0.003 | WRAP53 |
| Musculoskeletal | chr7_64863392_C_T | rs10265083 | 0.019 | 0.006 | ZNF92 |
| Multiple (any grade) | chr4_114278277_C_T | rs3733617 | 0.029 | 0.016 | ANK2 |
| Multiple (any grade) | chr12_48888594_C_T | rs11458 | 0.034 | 0.016 | C12orf54 |
| Multiple (any grade) | chr18_25616451_A_T | rs1944294 | 0.028 | 0.005 | CDH2 |
| Multiple (any grade) | chr19_41117869_A_G | rs1131620 | 0.028 | 0.009 | LTBP4 |
| Multiple (any grade) | chr7_99696797_T_C | rs2070215 | 0.041 | 0.007 | MCM7 |
| Multiple (any grade) | chr12_57619362_G_A | rs10783816 | 0.032 | 0.009 | NXPH4 |
| Multiple (any grade) | chr17_3119767_C_T | rs769427 | 0.032 | 0.016 | OR1A1 |
| Multiple (any grade) | chr6_144081609_C_T | rs2073214 | 0.028 | 0.012 | PHACTR2 |
| Multiple (any grade) | chr12_14720554_G_T | rs1141509 | 0.046 | 0.008 | PLBD1 |
| Multiple (any grade) | chr1_248039451_C_T | rs3811444 | 0.028 | 0.007 | TRIM58 |
| Multiple (grade ≥2) | chr11_134856603_C_T | rs117318814 | 0.017 | 0.000 | AK130852 |
| Multiple (grade ≥2) | chr4_114278277_C_T | rs3733617 | 0.017 | 0.001 | ANK2 |
| Multiple (grade ≥2) | chr2_241451351_G_A | rs35044862 | 0.013 | 0.001 | ANKMY1 |
| Multiple (grade ≥2) | chr18_76829525_A_G | rs4078115 | 0.017 | 0.003 | ATP9B |
| Multiple (grade ≥2) | chr5_114607217_T_A | rs77125878 | 0.014 | 0.011 | CCDC112 |
| Multiple (grade ≥2) | chr5_118506685_G_A | rs9790916 | 0.019 | 0.002 | DMXL1 |
| Multiple (grade ≥2) | chr21_37617724_C_A | rs3746866 | 0.015 | 0.004 | DOPEY2 |
| Multiple (grade ≥2) | chr4_89577214_A_T | rs2972040 | 0.015 | 0.001 | HERC3 |
| Multiple (grade ≥2) | chr5_7870973_A_G | rs1801394 | 0.013 | 0.004 | MTRR |
| Multiple (grade ≥2) | chr11_48238549_G_A | rs11606506 | 0.020 | 0.007 | OR4B1 |
| Neurologic | chr3_100558416_T_C | rs9841585 | 0.016 | 0.005 | ABI3BP |
| Neurologic | chr19_5772897_C_T | rs78536254 | 0.020 | 0.000 | CATSPERD |
| Neurologic | chr5_118506685_G_A | rs9790916 | 0.017 | 0.002 | DMXL1 |
| Neurologic | chr2_234750542_G_C | rs3732215 | 0.023 | 0.002 | HJURP |
| Neurologic | chr1_6157434_C_T | rs2311802 | 0.020 | 0.013 | KCNAB2 |
| Neurologic | chr13_44457925_A_G | rs3764147 | 0.019 | 0.001 | LACC1 |
| Neurologic | chr1_222803204_A_G | rs2936051 | 0.015 | 0.002 | MIA3 |
| Neurologic | chr15_42143533_T_G | rs1197665 | 0.018 | 0.001 | SPTBN5 |
| Neurologic | chr16_2812939_C_A | rs2240140 | 0.016 | 0.006 | SRRM2 |
| Neurologic | chr17_15611495_T_C | rs3760299 | 0.015 | 0.007 | ZNF286A |
| Pulmonary | chr6_109894773_T_A | rs12175588 | 0.004 | 0.002 | AK9 |
| Pulmonary | chr2_228493211_G_A | rs28739019 | 0.004 | 0.001 | C2orf83 |
| Pulmonary | chr14_24679877_C_T | rs2295322 | 0.004 | 0.000 | CHMP4A |
| Pulmonary | chr2_220037666_A_G | rs1127102 | 0.004 | 0.001 | CNPPD1 |
| Pulmonary | chr14_55655692_C_T | rs2274271 | 0.004 | 0.004 | DLGAP5 |
| Pulmonary | chr19_54758504_C_G | rs117301131 | 0.005 | 0.004 | LILRB5 |
| Pulmonary | chr8_125568175_T_C | rs3793395 | 0.004 | 0.002 | MTSS1 |
| Pulmonary | chr14_60585131_A_G | rs308998 | 0.004 | 0.000 | PCNXL4 |
| Pulmonary | chr7_140080087_C_G | rs62490396 | 0.006 | 0.001 | SLC37A3 |
| Pulmonary | chr8 135649848 G A | rs12541381 | 0.005 | 0.001 | ZFAT |
| Skin | chr7_150733025_T_A | rs2303926 | 0.056 | 0.001 | ABCB8 |
| Skin | chr12_52306221_C_T | rs2277382 | 0.034 | 0.036 | ACVRL1 |
| Skin | chr15_34159986_T_C | rs2241617 | 0.043 | 0.014 | AVEN |
| Skin | chr14_74489735_G_A | rs3784039 | 0.049 | 0.058 | CCDC176 |
| Skin | chr3_128204693_G_C | rs78245253 | 0.035 | 0.043 | GATA2 |
| Skin | chr9_712156_T_G | rs912174 | 0.051 | 0.018 | KANK1 |
| Skin | chr19_51361472_C_A | rs2003783 | 0.039 | 0.025 | KLK3 |
| Skin | chr12_31288955_G_A | rs11051266 | 0.035 | 0.002 | OVOS2 |
| Skin | chr1_227935762_G_A | rs2236358 | 0.051 | 0.009 | SNAP47 |

20. The method of claim 1, wherein the biological sample is one or more selected from the group consisting of tissue, cells, whole blood, serum, plasma, saliva, sputum, cerebrospinal fluid, urine, and feces.

21. A method, comprising:
measuring one or more activities selected from the group consisting of B cells, regulatory T cells, and exhausted T cells in a biological sample isolated from a subject;
predicting a high risk of occurrence of cancer immunotherapy-induced irAEs when the B cell activity is relatively high, or the regulatory or exhausted T cell activity is relatively low; and
when a high risk of occurrence of cancer immunotherapy-induced irAEs is predicted, treating the cancer immunotherapy-induced irAEs with a corticosteroid or an immunosuppressant.
